SEQUENCE LISTING (18 sequences)

SEQ ID NO: 1 - DNA, 25 bp, Artificial Sequence
  c-Met target(ch.7)
ccatcctaactagtggggactctga

SEQ ID NO: 2 - DNA, 21 bp, Artificial Sequence
  probe of reference gene 1
cacagtttgtcagcccttgga

SEQ ID NO: 3 - DNA, 20 bp, Artificial Sequence
  probe of reference gene 2
acggccagcgaagtgagttc

SEQ ID NO: 4 - DNA, 21 bp, Artificial Sequence
  probe of reference gene 3
tccaacctctgcctcaagatc

SEQ ID NO: 5 - DNA, 26 bp, Artificial Sequence
  probe of reference gene 4
ggaattgtcaaactgactccttttcc

SEQ ID NO: 6 - DNA, 20 bp, Artificial Sequence
  probe of reference gene 5(ch.7)
ttggatggcaagcatgtggt

SEQ ID NO: 7 - DNA, 24 bp, Artificial Sequence
  c-Met target F1
aagtgcagtatcctctgacagaca

SEQ ID NO: 8 - DNA, 23 bp, Artificial Sequence
  c-Met target R1
tggacagtattttgcagtaatgg

SEQ ID NO: 9 - DNA, 21 bp, Artificial Sequence
  probe of reference gene 1 F1
ccacatgctaactcgatatgc

SEQ ID NO: 10 - DNA, 24 bp, Artificial Sequence
  probe of reference gene 1 R1
gtggaagactgttcaacacttacc

SEQ ID NO: 11 - DNA, 18 bp, Artificial Sequence
  probe of reference gene 2 F1
tgccggagcttggaacag

SEQ ID NO: 12 - DNA, 21 bp, Artificial Sequence
  probe of reference gene 2 R1
aaaatgggcggaggagagtag

SEQ ID NO: 13 - DNA, 20 bp, Artificial Sequence
  probe of reference gene 3 F3
cagtgtgtgcaggtgaagaa

SEQ ID NO: 14 - DNA, 22 bp, Artificial Sequence
  probe of reference gene 3 R2
tgttaatgccaccaagtttgac

SEQ ID NO: 15 - DNA, 24 bp, Artificial Sequence
  probe of reference gene 4 F1
attttaggatgtttttcgcatctg

SEQ ID NO: 16 - DNA, 21 bp, Artificial Sequence
  probe of reference gene 4 R1
tggcagaatttgttcctatgc

SEQ ID NO: 17 - DNA, 23 bp, Artificial Sequence
  probe of reference gene 5 F3
gttgccagtcatagtgattgttc

SEQ ID NO: 18 - DNA, 20 bp, Artificial Sequence
  probe of reference gene 5 R3
ggcctccacaatattcatgc

(19) United States
(12) Patent Application Publication
(10) Pub. No.: US 2024/0182975 A1
(43) Pub. Date: Jun. 6, 2024

(54) METHOD FOR SELECTING NORMALIZING GENES FOR COPY NUMBER VARIATION DETECTION AND COMPOSITION FOR DETECTING C-MET COPY NUMBER VARIATION SELECTED BY METHOD

(71) Applicants: LOGONE BIO CONVERGENCE RESEARCH FOUNDATION, Seoul (KR); ABION INC., Seoul (KR)

(72) Inventors: Sung Su KIM, Seoul (KR); Yun Jung JUNG, Seoul (KR); Jun Young CHOI, Seoul (KR); Kyung Eui PARK, Seoul (KR)

(21) Appl. No.: 17/801,979

(22) PCT Filed: Feb. 24, 2021
(86) PCT No.: PCT/KR2021/002291

§ 371 Date: Aug. 24, 2022

(30) Foreign Application Priority Data
Feb. 24, 2020 (KR) 10-2020-0022590

(51) Int. Cl. C12Q 1/6886

(52) U.S. Cl. CPC C12Q 1/6886; C12Q 2600/118; C12Q 2600/156; C12Q 2600/158

(57) ABSTRACT

The present invention relates to a primer and probe set composition for CNV detection of a c-Met gene, and a kit comprising the same. By using the primer and probe set composition for CNV detection according to the present invention, it is possible to predict a prognosis of a c-Met-expressing cancer patient or a risk of mutation occurrence and to provide information required for targeted therapy of cancer, so as to be effectively used to provide clues for future treatment directions, as well as to determine a need to administer an anticancer drug, and to monitor the metastasis or recurrence of cancer.

### TECHNICAL FIELD

This application claims the priority of Korean Patent Application No. 10-2020-0022590, filed on Feb. 24, 2020, the entirety of which is a reference of the present application.

The present invention relates to a method for selecting normalizing genes for copy number variation detection and a composition for detecting c-Met copy number variation selected by the method, and more specifically, to a method for selecting 3 to 6 normalizing genes for copy number variation detection from a normalizing gene candidate group and a primer/probe set composition for detecting c-Met copy number variation using the method, a kit for detecting c-Met copy number variation comprising the composition, and a method of providing information required for prognostic evaluation of cancer, prediction of a risk of mutation occurrence, or targeted therapy using the composition.

### BACKGROUND ART

A genome refers to all genetic information of an organism. For sequencing of a genome of any one individual, various technologies such as DNA chips, next generation sequencing (NGS) technology, next next generation sequencing (NNGS) technology, and the like have been developed. Analysis of genetic information such as nucleic acid sequences and proteins is widely used to find genes that express diseases such as diabetes and cancer, or to determine a correlation between genetic diversity and expression characteristics of individuals, and the like. In particular, genetic data collected from individuals is important in identifying the genetic characteristics of individuals related to different symptoms or disease progression. Therefore, genetic data such as nucleic acid sequence, protein, etc. of the individual is key data for preventing diseases or selecting an optimal treatment method at an early stage of disease by identifying current and future disease-related information. Technologies have been studied to accurately analyze individual genetic data and diagnose individual diseases by using genome detection equipment that detects single nucleotide polymorphism (SNP), copy number variation (CNV), and the like as genetic information of living things.

The copy number variation (CNV) corresponds to structural variation in the individual variation of the human genome and has been predicted to describe a major part of polymorphism in human genes and to play an important role in genetic susceptibility to common diseases. The copy number variation is one type of promising structural modifications beyond single nucleotide polymorphism (SNP) affecting only one base. The copy number variation varies in size from 1 kilobase to several megabases.

Currently, the development of methods for diagnosing diseases through CNV detection of various genes has been widely studied, but no clear results have been yet obtained. One reason thereof is that standardization methods for detecting the copy number variation are not accurate.

In some documents (Cancer res (2007) March 1:67(5):2081-8), in a lung cancer cell line, a qPCR reference gene to identify MET amplification may be used, but these reference genes have limitation to quantitatively analyze the copy number by merely confirming the normal progress of the experiment.

### DISCLOSURE

### Technical Problem

Therefore, the present inventors have studied a method capable of rapidly and quantitatively calculating copy number variation occurring in many types of cancers, and then developed a method for selecting reference genes for detecting copy number variation (CNV) for a specific gene from a normalizing gene group used commonly in the related art. In addition, the present inventors demonstrated the method by rapidly detecting CNV of an overexpressed c-Met gene, and developed a kit suitable therefor and a method for providing information required for prognostic evaluation of cancer, prediction of a risk of mutation occurrence, or targeted therapy using the same, and then completed the present invention.

An object of the present invention is to provide a method for selecting normalizing genes for copy number variation detection by selecting 3 to 6 normalizing genes from a normalizing gene group.

Another object of the present invention is to provide a primer and probe set composition for detecting c-Met copy number variation (CNV).

Yet another object of the present invention is to provide a kit for detecting c-Met CNV comprising a primer and probe set composition for detecting c-Met CNV as an active ingredient.

In addition, the present invention is to provide a kit for detecting c-Met CNV consisting of a primer and probe set composition for detecting the c-Met CNV.

In addition, the present invention is to provide a kit for detecting c-Met CNV essentially consisting of a primer and probe set composition for detecting the c-Met CNV.

Still another object of the present invention is to provide a method for providing information required for prognostic evaluation of cancer or prediction of a risk of mutation occurrence using a primer and probe set composition for detecting c-Met copy number variation.

Still another object of the present invention is to provide a method for providing information required for targeted therapy of cancer using a primer and probe set composition for detecting c-Met copy number variation.

Still yet another object of the present invention is to provide uses of a primer/probe set for detecting c-Met copy number variation for preparing a primer and probe set formulation for detecting c-Met copy number variation.

Still yet another object of the present invention is to provide a method for detecting c-Met copy number variation (CNV) including the steps of:
(a) administering a primer and probe set for detecting c-Met copy number variation to a target gene for copy number variation detection in a sample;
(b) measuring the copy number of a reference gene set of the present invention;
(c) calculating a mean value of the copy numbers of each gene in the reference gene set;
(d) calculating a value by dividing the copy number of a target gene for copy number variation detection by the mean value calculated in step (c); and
(e) comparing the value calculated in step (d) with 2 and determining that the copy number variation exists when the value is greater than 2.

### Technical Solution

In order to achieve the object, the present invention provides a method for selecting normalizing genes for copy number variation detection by selecting 3 to 6 normalizing genes from a normalizing gene group.

In order to achieve another object of the present invention, the present invention provides a primer and probe set composition for detecting c-Met copy number variation (CNV).

In order to achieve yet another object of the present invention, the present invention provides a kit for detecting c-Met CNV comprising a primer and probe set composition for detecting the c-Met. CNV as an active ingredient.

In addition, the present invention provides a kit for detecting c-Met CNV consisting of a primer and probe set composition for detecting the c-Met CNV.

In addition, the present invention provides a kit for detecting c-Met CNV essentially consisting of a primer and probe set composition for detecting the c-Met CNV.

In order to still another object of the present invention, the present invention provides a method for providing information required for prognostic evaluation of cancer or prediction of a risk of mutation occurrence using a primer and probe set composition for detecting c-Met copy number variation.

In order to still another object of the present invention, the present invention provides a method for providing information required for targeted therapy of cancer using a primer and probe set composition for detecting c-Met copy number variation.

In order to still another object of the present invention, the present invention provides uses of a primer/probe set for detecting c-Met copy number variation for preparing a primer and probe set formulation for detecting c-Met copy number variation.

In order to achieve still another object of the present invention, the present invention provides a method for detecting c-Met copy number variation (CNV) comprising the steps of:
(a) administering a primer and probe set for detecting c-Met copy number variation to a target gene for copy number variation detection in a sample;
(b) measuring the copy number of a reference gene set of the present invention;
(c) calculating a mean value of the copy numbers of each gene in the reference gene set;
(d) calculating a value by dividing the copy number of a target gene for copy number variation detection by the mean value calculated in step (c); and
(e) comparing the value calculated in step (d) with 2 and determining that the copy number variation exists when the value is greater than 2.

Unless otherwise defined, all technical and scientific terms used herein have the same meaning as commonly understood by those skilled in the art. The following references provide one skill with general definitions of various terms used in the present specification: Singleton et al., DICTIONARY OF MICROBIOLOGY AND MOLECULAR BIOLOTY (2th ed. 1994); THE CAMBRIDGE DICTIONARY OF SCIENCE AND TECHNOLOGY (Walkered., 1988); and Hale & Marham, THE HARPER COLLINS DICTIONARY OF BIOLOGY.

Hereinafter, the present invention will be described in detail.

The present invention provides a method for selecting normalizing genes for copy number variation detection comprising:
(a) selecting a normalizing gene candidate group;
(b) obtaining variation information in target genes in the candidate group and determining whether or not pathogenic variation is present;
(c) calculating a variation rate of the pathogenic variation and selecting the target genes having a variation rate of less than 0.001;
(d) excluding genes corresponding to following criteria from the target genes in the candidate group,
(i) the presence of deep deletion, truncating mutation, and missense mutation
(ii) the presence of gene amplification in cancer cells; and
(e) selecting 3 to 6 normalizing genes from the normalizing gene group selected in steps (b) to (d), thereby selecting normalizing genes for copy number variation detection,
wherein at least one of the normalizing genes for copy number variation detection is located on the same chromosome as the target gene to detect the copy number variation.

In the present invention, the ‘copy number variation (CNV)’ refers to a DNA fragment showing a difference in the copy number found by comparing two or more genomes, and may be newly generated or inherited. About 350,000 CVN loci have been reported in the human genome, and exist in a size of about 40 to 100 kb, and the CNV region occupies about 600 Mb (to 2%) in the human genome. These CNVs (insertion, deletion, and duplication) affect a phenotype and the occurrence of cancer. During the occurrence of cancer, variation in a specific gene does not occur at once, but occurs in several stages. For example, in the occurring stage of colon cancer, variants in several genes occur in normal cells, resulting in mucosal hyperproliferation and adenoma, and then through mutations in major cancer suppressor genes, colon cancer finally occurs. Major genes are involved in each cancer type, and if the variations of these genes are determined, the stage of cancer progression may be determined. In addition, even in cancer of the same organ, since all gene variations are different (heterogeneity), the prognosis, treatment, and recurrence of cancer may be applied differently according to these differences.

The method for selecting reference genes for copy number variation (CNV) detection of the present invention will be described as follows for each step.

Step (a): (a) selecting the normalizing gene candidate group;

In the present invention, the normalizing gene candidate group may include other normalizing genes conventionally used for research or diagnostic purposes. For example, since GAPDH or ACTB is a normalizing gene widely used in molecular biology experiments, the normalizing gene may be included in the normalizing gene candidate group of the present invention. Preferably, the normalizing gene candidate group may be a group consisting of 10 to 37 genes selected from the group consisting of 18S, ACTB, ALDOA, ARHGDIA, AP3B1, B2M, GAPDH, GUSB, HMBS, HPRT1, HSPCB, IP08, LDHA, NONO, PGK1, POLR2A, PPIA, RPL11, RPL19, RPL32, PLP0, RPS18, RPS27A, EIF2C1, SFRS9, TBP, TFRC, UBC, YWHAZ, RNaseP, TERT, LINE-1, MTHFR, RPPH1, HBB, TUBB, and PPIH, much more preferably a group consisting of 20 to 37 and 30 to 35 genes, and most preferably a group consisting of the 37 genes.

Information on each gene included in the normalizing gene candidate group is shown in Table 1 below.

TABLE 1

| Position of | | |
|---|---|---|
| chromosome | Gene name | Description |
| 13, 14, 15, 21, 22 (5 | 18S | 18S ribosomal RNA |
| clusters | | |
| Chr7.p22.1 | ACTB | actin, beta |
| 16 | ALDOA | aldolase A, fructose-bisphosphate |
| 17 | ARHGDIA | Rho GDP dissociation inhibitor |
| | | (GDI) alpha |
| 5 | AP3B1 | adaptor related protein complex 3 |
| | | subunit beta 1 |
| 15 | B2M | beta-2-microglobulin |
| 12 | GAPDH | glyceraldehyde-3-phosphate |
| | | dehydrogenase |
| 7 | GUSB | glucuronidase, beta |
| 11 | HMBS | hydroxymethylbilane synthase |
| X | HPRT1 | hypoxanthine |
| | | phosphoribosyltransferase 1 |
| 12 | HSPCB | heat shock 90 kDa protein 1, beta |
| 12 | IPO8 | importin 8 |
| 11 | LDHA | lactate dehydrogenase A |
| X | NONO | non-POU domain containing, |
| | | octamer-binding |
| X | PGK1 | phosphoglycerate kinase 1 |
| 17 | POLR2A | polymerase (RNA) II (DNA directed) |
| | | polypeptide A |
| 7 | PPIA | peptidylprolyl isomerase A |
| | | (cyclophilin A) |
| 1 | RPL11 | ribosomal protein L11 |
| 17 | RPL19 | ribosomal protein L19 |
| 3 | RPL32 | ribosomal protein L32 |
| 12 | RPLP0 | ribosomal protein, large, P0 |
| 6 | RPS18 | ribosomal protein S18 |
| 2 | RPS27A | ribosomal protein S27a |
| 1 | EIF2C1 | Anti-Eukaryotic translation initiation |
| | | factor 2C |
| 12 | SFRS9 | splicing factor, arginine/serine-rich 9 |
| 6 | TBP | TATA box binding protein |
| 3 | TFRC | transferrin receptor |
| 12 | UBC | ubiquitin C |
| 8 | YWHAZ | 3-monooxygenase/tryptophan 5- |
| | | monooxygenase activation protein, |
| | | zeta polypeptide |
| 14 | RNaseP | Ribonuclease P |
| 5 | TERT | telomerase reverse transcriptase |
| 7 | LINE-1 | Retrotransposon |
| 1 | MTHFR | methylenetetrahydrofolate reductase |
| 14 | RPPH1 | Ribonuclease P RNA component H1 |
| | | (H1RNA) gene (RPPH1) |
| 11 | HBB | Beta-globin |
| 6 | TUBB | Beta-tubulin |
| 1 | PPIH | Cyclophilin H |

Step (b): obtaining variation information in a target gene in the candidate group and determining whether or not pathogenic variation is present;

Step (c): (c) calculating a variation rate of the pathogenic variation and selecting the target gene having a variation rate of less than 0.001;

Steps (b) and (c) are steps for excluding genes corresponding to the pathogenic variation by discriminating pathogenic variations of the genes in the normalizing gene candidate group, and selecting genes with no or few pathogenic variations.

The pathogenic variation is a phenomenon in which the original pathogenicity is changed, and the determination of the pathogenic variation is performed by determining whether clinical information (Clin. Sig.) of each gene provided by, for example, Ensembl (http://ensembl.org) corresponds to ‘pathogenic’, ‘likely pathogenic’, or ‘likely benign to pathogenic’. When the variation corresponding thereto has a frequency (gmaf_freq) of 0.01 or more, it is determined as the pathogenic variation.

Meanwhile, it is determined that genes with no basis for pathogenic variations do not correspond to pathogenic variations. When there are many pathogenic variations, the likelihood of developing hereditary cancer is very high.

Step (d): excluding genes corresponding to the following criteria from the target genes in the candidate group;

Step (d) is a step of excluding genes inappropriate as normalizing genes for copy number variation detection, and cases of (i) the presence of deep deletion, truncating mutation, and missense mutation and (ii) the presence of gene amplification in cancer cells are excluded from the normalizing genes for copy number variation detection.

In the present invention, deep deletion indicates that deletion mutation occurs in the corresponding gene or gene pooling in all homologous genes, that is, a value obtained by calculating a copy-ratio (log 2 (copy number)) of the specific region of the gene is referred to as −2 (homozygous deletion).

When variations of deep deletion, truncating mutation, and missense mutation as described above appear in the reference genes, normal gene amplification does not occur because primers and probes for detecting the reference genes are non-specifically bound to each other or not bound to each other, thereby making it impossible to accurately detect the copy number.

In addition, when there is abnormal overamplification of the reference gene, it cannot be seen that there are characteristics of the reference gene that is always constantly expressed (e.g., there are copy numbers of two reference genes per cell), so that it is impossible to detect the copy number variation of a target gene. In other words, even if the copy number of the actual target gene is abnormal, since the target gene is compared with the reference gene, it is impossible to detect exact copy number variation of the target gene when the copy number of the reference gene is abnormal.

Step (e): selecting normalizing genes for copy number variation detection by selecting 3 to 6 normalizing genes from the normalizing gene group selected in steps (b) to (d);

Step (e) is a step of selecting 3 to 6 genes as normalizing genes to be selected in steps (b) and (c) and used for normalization in the normalizing gene group excluding inappropriate genes in step (d). Since detailed analysis conditions may vary for each institution conducting genetic analysis and differences in condition and quality of a sample to be analyzed may affect the analysis results, in order to minimize these variables, the quality of the sample to be analyzed may be quantified and normalized. In addition, a result of quantifying how much the copy number has been amplified or decreased in the level of CN, that is, copy number variation, may be obtained through normalization.

The normalization result is calculated as a mean value of the measured values of each of 3 to 6 selected genes, and the mean may be calculated by an arithmetic mean, a geometric mean, a harmonic mean, a power mean, a weighted arithmetic mean, or a combination thereof. Preferably, the mean may be calculated by the arithmetic mean. In addition, at least one of the normalizing genes for the copy number variation detection is located on the same chromosome as the target gene to detect the copy number variation. In this way, the copy number variation (CNV) caused by abnormality in chromosome number may also be detected.

The CNV may be preferably CNV associated with a specific disease, and the specific disease may be cancer or tumor disease. Accordingly, in the present invention, the target gene for CNV detection may be preferably a target gene associated with cancer or tumor. In the present invention, the target gene may be c-Met, HER2, HER3, HER4, EGFR, KRAS, NRAS, ALK, RAC1, NTRK1˜3, NOTCH1-3, HRAS, HOXA3, DDR2, FGF3, AXIN1, INHBA, RET, FGFR3, MAP2K2, EPHA5, c-Myc, ABL1, ACTN4, AKT1, AKT2, AKT3, APC, ARAF, ARID1A, ARID2, ATM, AXIN1, AXL, BAP1, BARD1, BCL2L11, NRAF, BRCA1, BRCA2, CCND1, CDK4, CDKN2A, CDK4, CRKL, CREBBP, CTNNB, CUL3, ENO, EP300, ESR1, EZH2, FBXW7, FGFR1˜4, FLT3, GNA11, GNAQ, GNAS, ODH1, IDH2, IGF1R, IGF2, IL7R, JAK1˜3, KDM6A, KEAP1, KIT, MDM2, MDM4, MLH1, MTOR, MSH2, NF1, NFE2L2, NRG1, NT5C2, PALB2, PBRM1, PDGFRA, PDGFRB, PIK3CA, PIK3R, PIK3R2, POLD1, PTCH1, PTEN, RAC1, RAD51C, RAF1, RB1, RET, RHOA, ROS1, SETBP1, SETD2, SMAD4, SMARCA4, SMARCB1, SMO, STAT3, STK11, TP53, TSC1, and VHL, preferably c-Met.

Meanwhile, the present invention provides a reference gene set for copy number variation (CNV) detection by the method. The reference gene set for copy number variation (CNV) detection of the present invention may be selected from the group consisting of 18S, ACTB, ALDOA, ARHGDIA, AP3B1, B2M, GAPDH, GUSB, HMBS, HPRT1, HSPCB, IP08, LDHA, NONO, PGK1, POLR2A, PPIA, RPL11, RPL19, RPL32, PLP0, RPS18, RPS27A, EIF2C1, SFRS9, TBP, TFRC, UBC, YWHAZ, RNaseP, TERT, LINE-1, MTHFR, RPPH1, HBB, TUBB, and PPIH, and preferably consists of 3 to 6 genes, but may consist of 2 to 8 genes, 3 to 8 genes, 3 to 7 genes, 3 to 6 genes, 3 to 5 genes, and 3 to 4 genes.

The reference gene set for copy number variation detection of the present invention may further include one or more housekeeping genes. The housekeeping genes may be housekeeping genes known in the art, and for example, may be genes selected from the group consisting of PRDX6, SULT1A1, BZRP, ATP5G3, CSTB, HNRPF, MAPK3, SNRPC, TGFB1, TCFL1, ALDH9A1, LDHA, PCBP1, KARS, MRPL23, G3BP, ADFP, STOML2, FUSIP1, PLXNB2, KLF6, GPAA1, DKC1, FAM32A, CCNL1, RBM23, TMOD3, TM4SF8, MGC10433, DNCL1, ILK, FLJ20643, MAGED1, ZDHHC4, ADIPOR1, UBA52, C2orf25, RNPEPL1, GNB2L1, CREG1, CNAP1, FBXO7, RAB8A, JTB, RGS19IP1, GPR172A, ATP6AP1, SFRS6, ANAPC5, NOSIP, ATP6V0B, DKFZP566E144, NDUFV1, CALU, FIBP, PNRC2, RYBP, HIG1, RPS20, UQCR, WDR6, TMEM4, DDX42, CANT1, CYR61, FLJ13868, MGC52000, C14orf120, NIFIE14, NME4, C2orf28, SEC11L1, ABCF1, UBQLN1, NDFIP1, CGI-128, DCXR, COPE, RAN, BMS1L, SPCS1, UBE2R2, IDH1, TMPO, UMP-CMPK, ABCE1, TUFM, SNX3, BC-2, WDR39, KIAA1033, SRPRB, BECN1, ADAR, NUP133, NSFL1C, MGC5508, NOLA3, FLJ10349, C10orf9, POLR2G, SLC7A1, TGOLN2, C16orf33, COPS6, RAB7, HSPC009, E2-230K, KIAA0431, FLJ11730, MGC4767, FUCA2, FLJ12892, PPM1G, LGMN, C20orf44, MRPL15, MAF1, P29, KNS2, CDK5RAP3, HRMT1L2, LOC127262, IGF1R, TIMM17A, DENR, UBE3A, CYBASC3, KIAA0664, LOC92912, FARSLA, SAFB, POLR2E, TRAPPC1, FBLN1, RGS5, NET1, SLC29A1, MTVR1, PFKP, FOXJ3, MGC14156, MAN2C1, GSTZ1, TM4SF7, NOLA2, SAT, APRT, GBL, CLSTN1, MGC2963, HSPC182, TRAP1, PMVK, CKAP1, C20orf14, DKFZP564J0123, CDCA4, TAX1BP1, BLOC1S2, TEGT, MARK3, PPP1R7, HSPH1, STRA13, DPP7, Cab45, MTPN, POLR21, NDUFA4, HIP2, TEBP, HSPBP1, FAM50A, CD81, MAT2B, RY1, EIF3S7, MRPL51, CTNNAL1, SMC4L1, HSDL2, PTPN12, DDX27, RNF40, BPY2IP1, C22orf16, SFRS1, HEXB, RNF126, UNR, SQLE, MRPS7, CSNK2B, APEX1, GNAI3, SFRS2, ETFB, FVT1, TMP21, DVL1, YWHAQ, GJA1, OAZ2, SSR2, GDI1, TIMM13, MARCKSL1, TSN, FASTK, ILF2, TFAM, ARFIP2, DAP, NDUFA9, BRD2, ARL6IP, IRF3, TUBA1, PSME1, QDPR, ADSL, COPB2, C20orf111, C12orf8, MBTPS1, RNP24, DAG1, ECHS1, UBL4, ZDHHC16, GPX1, GANAB, PSMB6, GNA12, CDK10, PLP2, HMGN3, USP9X, CSK, SF3A3, HLA-B, DKFZp76112123, PSMD8, UROD, PGK1, ILVBL, DBI, ADH5, DHPS, PPP1CC, RCN2, CCNG1, NCL, QARS, SMARCD1, PSMC5, POLR2C, RPA2, C20orf149, RPL37, SYPL, ATP5G1, NFKBIA, SUMO1, RAD21, SEC24C, CSNK2A2, GSS, MGC21416, PSMB3, PPP1R11, DAD1, CCT6A, AMPD2, FASN, AAMP, PRDX4, STX4A, SNRPB, DHFR, NDUFA5, GABARAP, FL112442, ZFP36L1, ERBP, DERPC, FADD, MSN, PSMB4, TKT, EPHX1, UBTF, SKIV2L, PGRMC1, HSPA4, ADRM1, NDUFS8, KHSRP, MLLT6, ERP70, LOC54499, CDK4, COX11, FLJ21128, HNRPA0, SH13GL1, RCN1, FUBP3, ACTR1B, MCTS1, PSMA1, ARL6IP4, MGC5576, TIMP2, NPDC1, RALGDS, ATP6V0D1, ANAPC13, CCNB1IP1, FLJ31031, C7orf20, C3orf4, SH3BGRL, CSRP1, E2F4, APH-1A, RPS27L, PTD008, SH3BGRL3, C14orf2, SIAT7F, C6orf48, SF3B5, ESRRA, MRPS11, ITM2C, ARS2, SIVA, TOMM7, NUDT5, SSR1, CD9, TXNL1, RFC3, EXOSC7, GLS, PABPN1, ST5, BST2, FSCN1, PNPLA2, NME1, FUT8, p66alpha, GSDMDC1, ARF3, H2AFZ, UQCRC1, AP2S1, RPL3, DNAPTP6, NUP153, CDIPT, WIPI-2, SND1, ARPC1A, FBXL11, C10orf1119, BBX, CCT8, EVL, GNAS, AEBP2, RAMP, NAPA, DHX38, EAP30, MAMDC2, RAB5C, CTSC, SEPT11, WDR1, CINP, EIF4A1, TRIO, DDX23, CROP, TM9SF2, BNIP3L, PRNPIP, 132513 HSD17B12, TPM1, SLC35E1, PSMD13, CEBPZ, UREB1, RALY, NCOR2, ARHGAP1, MAEA, M6PRBP1, HP1-BP74, DDX49, DRPLA, S100A10, EBSP, MGC3234, EEF1G, VTI1B, MUF1, ZNF313, SCAMP4, PLEKHM2, MAP2K1, UBL3, TPM3, HERPUD1, QP-C, SPIN, CUL1, PCNA, RBAF600, CCM2, ARF4, PTPRG, RHOBTB1, FBXO31, RPS6KA1, PEX14, BIRC6, BC002942, SUI1, TXNDC5, OXA1, KIAA0992, GMFB, U5-116KD, p44S10, RPS28, TXNDC4, SLC35B1, ZFPL1, HNRPUL1, NFE2L2, KJAA0676, PSMC6, RPS29, KIAA1536, MRPS34, PTD004, H2AFV, HAGH, PRPF4B, AMD1, RAF1, ARHGDIA, FBXO21, THRAP2, CDC37, PDHB, HBP1, CHD4, PACSIN2, MOCS2, UBE2J1, PICALM, VAPA, CTNND1, PHF1, HNRPU, SEC13L1, SFRS5, SRP54, TRPC4AP, METTL3, DIABLO, CNN2, UQCRFS1, NFIC, CNOT7, CFL1, SKP1A, PTBP1, BRP44L, COL1A1, NYREN18, NOC4, DPYSL2, FKBP8, NDUFS2, LOC401152, CKB, EDF1, IFITM2, AKI, YT521, ATP5E, PARP1, RPL8, MBD3, FLOT1, CFL2, MRPS16, HSPA8, H3F3B, 384D8-2, PRDX1, CXXC1, DUSP3, MED4, HMGN2, HLA-A, PRPF8, TMEM9, NHP2L1, C22orf5, SLC35B2. EIF4G2, ADD1, RANGAP1, DCTD, PPP1CA, C20orf24, PMPCB, HSPA9B, ELAVL1, GNB2, SPG7, MALAT1, RPS15A, BRD4, SDFR1, SLC20A1, PAFAH1B2, PLEKHA5, RBM6, NUDT3, PTK9, CXXC5, SMURF1, NDUFAB1, CCT2, GSTO1, MRCL3, RAP1A, COPS4, HSPC142, STRN4, TTC17, SEPT7, DHX9, UBE2J2, CDC2L1, TRA1, EIF3S8, RAI17, BIN1, TUBB6, TCEAL4, ZNF265, FLNA, C17orf27, SSFA2, PKM2, RANBP2, HAX1, HDAC7A, SCAMP3, SDCBP, ch-TOG, WDR45L, GLG1, GK001, VDAC1, HNRPH1, SMN2, KIAA0261, SGTA, AHSA1, MEP50, MRPL3, CTTN, MGC71993, CTBP1, STMN1, ELMO2, KIAA0141, DNM2, PSMC4, NDUFS7, TXNDC7, CIZ1, NUCKS, PSMB7, KIAA0460, SUPT16H, FBXO9, FOXO3A, SLC38A2, DAZAP1, DDX21, SDC1, RDH11, H1F0, YWHAH, ATAD3A, TOMM70A, PTP4A1, PC4, PITPNC1, HSPC023, KIAA0174, PDCD4, NFYC, CDC2L5, PSMA7, MAPKAPK3, SMARCC2, MGC20781, XTP3TPA, SCYL1, MGC2731, ATPIF1, POLDIP2, ARIH2, GNPTG, DERL1, SERPINH1, SPG21, DGKI, ASCC3L1, ATP5J, CPNE1, FBXO1, RHOA, FBS1, HSPD1, MPST, TAF9, DLGAP4, ARL10C, SUPT6H, PSMC3, HSBP1, LOC51234, PSMA4, MVD, TTC15, DKFZp566C0424, PAPOLA, STOM, BAT1, VPS24, PFKL, XRN2, BTG1, CRI1, MGC13170, NUBP2, PLD3, SAV1, SH3BP5, DNPEP, FEZ2, C10orf86, PALM2-AKAP2, PIP5K2B, FLJ10326, ITGA3, KIAA1185, GPS1, C13orf12, GLO1, MATR3, MAK3, PTPRA, GNAQ, MTCH2, ACTN4, SUCLG1, LASS5, ZNF410, ATP5C1, NOB1P, PTPRF, TDE1, ATP6V1D, SEC23A, TMEM14B, TFE3, C15orf15, CARS, S100A6, SEPP1, PCNP, NUP93, MGLL, C11orf2, ARHGEF1, MEA, PDAP1, C9orf78, GNPDA1, SNX17, CD59, SLC39A7, C17orf15, TACC1, PCMT1, POLD1, PX19, DREV1, SEPX1L TPR, MRPL4, TUBG1, SUMF2, DDX5, COMMD9, YWHAB, TMED9, SBF1, PRKARIA, SNRPB2, CALM1, SPCS2, RNPC2, RBM9, C14orf124, BNIP2, RHEB, APG4B, IDI1, UBQLN4, ANKRD25, HMOX2, MRPS24, PDXK, MAX, LASS2, ARF1, MYO1C, KPNA4, PFDN5, HSCARG, C6orf68, COTL1, DCTN2, CYC1, GBF1, SLC25A3, DDB1, ARID1B, EIF4E2, EIF4B, LARP, ZMYND11, 292457, G22P1, CCNH, PTDSS1, FLJ12666, ATP6V1B2, TIMP3, CKLFSF3, ATP5A1, DUSP6, FBL, GDI2, RPL39, RCP9, TPM2, 300816 RAB1B, GALNT2, RBM3, Ufc1, MRPS6, UBE21, G3BP2, DSTN, CST3, MAGEF1, RANBP9, ZA20D3, IBTK, ITPK1, NUP188, GRP58, SFRS7, C6orf153, RNF11, STARD3NL, C14orf159, TOMM40, RAB1A, MRPS35, CMAS, DDX39, RPS27A, ADAM15, TXNDC11, BAZ2A, EIF3S12, IFRD2, GC20, NASP, MGC4549, RAB11A, APG9L1, FLJ20758, NDUFB2, VKORC1, EHD2, MORF4L2, CORO1C, SCC-112, EEF1D, HSPC152, PSMA2, MRPL13, K-ALPHA-1, TRAF7, GNS, RBPMS, BMSC-UbP, LASP1, PPP2R5E, ANKRD11, SEPT2, KIAA1102, FDPS, STIP1, TXNRD1, COPB, COX7A2L, GRINA, E124, KCMF1, EBNAIBP2, DR1, SCARB2, ZMAT2, CASC3, IRF2BP2, C9orf10OS, SLC25A6, FLJ10241, LOC51035, TM4SF1, PAQR4, COX6C, STCH, GHITM, PSMB1, POR, TNIP1, MGC23909, RNPS1, FLJ20507, MGC5306, DKFZp586M1819, MARS, VDAC2, SFPQ, BZW1, MAP1LC3B, FLJ10350, UBB, SDHD, HMGN1, PPIA, RPS2, RPL28, LOC149603, MGC2747, PHF6, RPLP1, SNRPD3, NUTF2, SNX6, PSMC1, MAN2B1, SOX4, SEP15, U2AF1, LRPPRC, CCT7, PYGB, DYRKIA, PPP2R5C, SRPR, LOC51337, EXT2, PDLIM1, LEREPO4, MGC40157, TRIP12, RAB2, SELT, DNCL12, PSMD14, DKFZP434B168, MLL5, CPSF6, GAK, COPS2, FLJ20551, DAZAP2, MGC2749, RAP1B, SEC31L1, APLP2, BCCIP, FNTA, COMT, CAP1, XPO1, CDKN1A, RPN2, PRO855, TAPBP, EIF3S9, CARM1, RAB14, DKFZP547E1010, ATP1A1, C9orf10, SMAP-5, CUL3, SPTAN1, KBTBD2, ARL1, NDRG1, TGIF, HM13, HNRPR, CAMTA2, VGLL4, ASXL1, SIAT4A, CKS1B, EWSR1, MORF4L1, RPL17, TPT1, IFITM3, PRPF4, TLN1, CD24, RNF31, BTN3A2, GUK1, LYRIC, RPS5, HMBS1L, eIF2A, PCGF4, DDX3X, RPL38, SUMO2, HLA-E, KIAA0146, PPIF, RPL21, RPS14, CBX3, 381219, GLTP, MRPS2, NCOA3, ANP32E, UBE2B, ANXA7, CHERP, USP7, FAU, PABPC1, RXRB, ATP6V1G1, RPL11, XRCC5, YY1, C19orf22, MCM3APAS, ATP6V0C, PINK1, DDX48, TCEAL5, CHCHD2, GSTK1, ACTR2, PTK2, PAIP2, NIP30, SLC9A3R1, RPS16, AKAP8L, RPL30, RBM10, COX5A, RPL10, STK24, EIF3S1, GARS, SFRS3, OGT, LOC284058, EIF3S6, MRPS21, LOC339229, NDUFA11, UBE2M, ZA20D2, SF3A1, RPL23, SF3B2, ATP5B, LOC389541, HMG20B, PGR1, RPS10, RPS15, RAB18, SLC35A4, C19orf13, PKP4, MIF, RPL36, RPS6, TXNL5, NDUFS6, KAB, FLJ10769, CHES1, SVIL, GOLPH3, ATP5O, SSR4, AGPAT1, PHPT1, IDH3G, HAN11, RPL13, AUP1, EIF4EBP1, MAPK6, EFHA1, ANXA6, ESRRBL1, AIP, KLHDC3, C10orf7, WBSCR22, C21orf33, SCOTIN, KIAA1049, TNPO2, TRIM50A, S100A11, DERP6, CTSL, VPS28, MRPL24, MRPL11, BUB3, ATP5D, PARK7, COL6A2, H2AFY, RPL7, CCT4, ZNF511, RDBP, TTC11, SERF2, LSM5, UBE2L6, C10orf104, DKFZp564J157, ITGB1, SEPN1, RTN4, PITPNA, MGC23908, GNB1, IQGAP1, CS, ARF5, CLNS1A, GNG12, C6orf55, FOXP1, MAP4K4, COX6B1, MAPK1, PPP5C, NFKB1, PRDX2, EML4, ESD, SLC39A9, CAPZB, RPL4, NR1H2, PLSCR3, CDK2AP1, NPC2, ARD1, BCKDHA, SRRM2, 433345, COX4I1, ACTR3, RPS11, DNAJC8, Bles03, TUBB2, RPL37A, KIAA1967, CLK1, EIF4G1, BANF1, SHC1, PBP, COX8A, GPX4, HMGB1, HARS2, ANKHD1, ZNF638, PPIB, HMGB2, APP, TBC1D22A, KIAA1608, KIF1C, TXN, LBR, ZFR, UBXD1, ACTL6A, PPP3CA, ZNF395, WAC, GCSH, THAP4, API5, TNRC15, LYPLA1, PHF10, ATAD1, CDK5RAP1, MTHFD1, TUBA6, BAT2, ZNF289, EMP1, IDH3B, ALDH2, ARMET, DBNL, CD74, POLR2F, CLU, SET, VBP1, SUPT5H, CYCS, ANXA2, ACADVL, GRINL1A, MGAT4B, GBAS, PIGT, PP, RPLP2, XBP1, C11orf10, C14orf32, UNC84A, GPS2, RPS19, TALDO1, MCM7, TBL1XR1, CUTL1, RBM5, SUPT4H1, FLJ22875, 439552, HBXIP, RFP, CLIC4, DDX1, PSMD7, TTYH3, SEPT9, RAD23A, CAST, POLR2L, C20orf22, IRS2, RNF10, GBA2, PSMD11, NPEPPS, SOD1, DKFZp761C169, GRB2, CTDSP1, SDHC, VMP1, CRR9, AZI2, CGGBP1, CRSP6, C2orf4, METAP2, NS5ATP13TP2, LGALS1, VPS29, SKIIP, RIOK3, CD63, DC2, KHDRBS1, GTF3A, WSB1, WTAP, CAPZA2, LDHB, PSMA6, PXN, FTH1, CD47, OAZ1, YIF1, ITM2B, TMSB10, RPS13, HNRPL, RPS4X, ARAF, EIF3S6IP, ST13, PGAM1, VPS35, RPLP0, NGFRAP1, RPL27A, HNRPC, HNRPK, USF2, IL6ST, PKD1-like, FLJ10597, DC12, TSPYL1, IFITM1, C19orf27, ANP32A, OAZIN, BTBD1, FLJ23790, AKAP13, MPG, CLCN7, PTMA, LITAF, SH3GLB2, ALS4, GGA2, XPO6, ATXN2L, LOC124446, CNOT1, GOT2, APPBP1, G6PD, CYB5-M, CFDP1, GABARAPL2, HSPC176, PCOLN3, CRK, RPA1, UBE2G1, RIP, UBE2S, TTC19, USP22, PIGS, PPARBP, IGFBP4, SMARCE1, FKBP10, RERE, STAT3, CDC27, AKAP1, BCAS3, C1orf33, PGD, ACOX1, SYNGR2, P4HB, AGTRAP, MRLC2, PPP4R1, TNFSF5IP1, P15RS, NARS, EFHD2, TXNL4A, MIDN, BTBD2, MAP2K2, C19orf10, HNRPM, PIN1, SDHB, PKN1, TPM4, NR2F6, GPI, SIRT2, LTBP4, SNRPA, EIF4G3, SNRP70, PPP2R1A, LENG4, RPS9, TRIM28, CDC42, HPCAL1, ODC1, LAPTM4A, PUM2, RAB10, PPP1CB, PIGF, CALM2, AFTIPHILIN, DGUOK, DKFZP564D0478, STARD7, RALB, YWHAZ, FLJ14346, SFRS4, YWHAE, ARL5, 470417, PTP4A2, EIF2S2, KPNA6, STAT1, SF3B1, NOP5/NOP58, NDUFS1, PSMB2, ACSL3, CAB39, MGC4796, M11S1, HDLBP, DTYMK, FKBP1A, C20orf116, PRNP, C20orf30, UBE2D3, CGI-94, SYNCRIP, MKKS, NDUFS5, RRBP1, C20orf3, MACF1, AK1P, SDBCAG84, BLCAP, TOP1, TPD52L2, NSEP1, GART, SLC2A1, RTN3, OTUB1, SUMO3, PTTG1IP, COL6A1, B4GALT2, UFD1L, AKR1A1, HSPC117, MYH9, CSNK1E, RUTBC3, SLC25A17, RBX1, ACO2, ATXN10, LRRFIP2, FLJ22405, LOC55831, RAB5A, TOP2B, SIMP, CTNNB1, TLP19, SMARCC1, IHPK2, IMPDH2, ALAS1, SCP2, FLNB, MRPL37, DKFZP5640123, DULLARD, ATP1B3, GYG, MBNL1, PA2G4, EIF4A2, FBXL5, LAP3, SLC30A9, SFRS11, GAPD, BCAR1, POLR2B, HNRPD, PDLIM5, SCYE1, ANXA5, UQCRH, MYO00, KAT3, RPL26, SLC30A5, TINP1, KIAA0372, PJA2, C5orf13, HINT1, PPP2CA, CNN3, JMJD1B, FBXW11, ATP6V0E, ETEA, DDX41, RNF130, CPM, DEK, RPL35, SORT1, PSMA5, MTCH1, AHCYL1, DST, BCLAF1, VIL2, TCP1, BZW2, HNRPA2B1, KIAA1068, OGDH, DKFZP564K0822, FLJ10099, BAZ1B, ASNS, ARPC1B, CPSF4, SYAP1, PBEF1, CALD1, SSBP1, ZYX, DNAJB6, CHR2SYT, MTX1, ELP3, LMNA, PPP2CB, CCT3, VDAC3, UBE2V2, TCEA1, TRAM1, WDR42A, LAPTM4B, EDD, EIF3S3, CGI-07, AK3L1, WDR40A, ANXA1, LAMP1, NINJ1, ANP32B, XTP2, PFN1, CDW92, FBXW2, NDUFA8, KIAA0515, PMPCA, CD99, MGC4825, ATP6AP2, PCTK1, DKFZp761A052, 496271, ATF4, IL13RA1, LAMP2, IVNS1ABP, WARS, C1orf48, ENAH, F11, ADSS, PNAS-4, KIAA0217, RBM17, DHCR24, YME1L1, GGA1, TIMM23, C10orf74, HNRPH3, VPS26, SARA1, PPP3CB, VCL, ENTPD6, GLUD1, IDE, SMBP, ZNF207, MGEA5, CUEDC2, ADD3, MXI1, TIAL1, BSG, CIRBP, PLEKHJ1, UROS, NCLN, IER2, NAP1L4, STIM1, C11orf15, USP47, MLSTD2, CAT, CD44, DGKZ, NDUFS3, C11orf31, RHOC, PRP19, FADS2, SLC3A2, MTCBP-1, PRDX5, SF1, ARL2, CAPN1, MAP3K11, RHOB, ZFP36L2, RAB6A, PME-1, HSPC148, PORIMIN, MGC2714, DARS, ITM1, RPS27, PTMS, REA, MYL9, DDX47, STRAP, KRAS2, PSMD4, C12orf10, COPZ1, CIP29, MYL6, PBXIP1, CGI-51, RARS, NUDT4, ATP2A2, DDX54, KIAA0152, FLJ12750, MGC9850, PFAAP5, PGRMC2, TGFB114, FNDC3A, FLJ10154, KIAA1181, FKBP3, KLHDC2, KTN1, RGL2, HSPCB, ERH, NUMB, ENSA, DDX24, MCP, FLJ10579, DKFZP564G2022, SNAP23, SRP9, TCF12, 511862, CBX6, ARPC3, SURF4, RPS17, MIR16, PRKCSH, KIAA1160, 512676, FLJ20859, THAP7, AP3D1, CD151, H63, ARPP-19, C15orf12, REC14, RANBP5, TMED3, MESDC1, RPL9, IDH2, NEUGRIN, FURIN, MRPL28, RHOT2, C16orf34, NDUFB10, NFATC2AP, MVP, ALDOA, BCKDK, FUS, ARL2BP, DPH2L1, FLII, MAP2K3, SDF2, FLOT2, JUP, RPL27, MGC4251, CGI-69, GRN, FLJ13855, PHB, SF3B3, WBP2, LGALS3BP, ACTG1, HGS, AP2B1, ATP5F1, NDP52, CAPZA1, C19orf6, STK11, GNA13, AES, EEF2, CLPP, MGC2803, CALR, GADD45GIP1, DNAJB1, LSM4, RENT1, SFRS14, RPL22, CAPNS1, AKT2, EGLN2, DEDD2, SNRPD2, SYMPK, CALM3, SLC1A5, SAE1, KDELR1, RPL18, NUCB1, PTOV1, LOC284361, PRPF31, PPP1R12C, GPSN2, BLVRB, RUVBL2, HADHB, YPEL5, TIA1, FL14668, TEX261, DCTN1, WBP1, MAT2A, FLJ20297, FL21919, HNRPA3, UBE2Q, NCKAP1, CHPF, ARHGEF2, STK25, TRIB3, CENPB, SLC35C2, CEBPB, C20orf43, ENO1, TAGLN2, PEA15, PEX19, IFNGR2, SON, F11R, ATP6V1E1, DEDD, COL18A1, DGCR2, PCQAP, ASCC2, EP300, PES1, MCM5, UNC84B, L3MBTL2, DIA1, PP2447, DKFZP564B167, C3orf10, MGC3222, 517821, CRTAP, DHX30, MAP4, APEH, TUSC2, ARL61P5, TFG, SEC61A1, RPN1, ZNF9, COPG, H41, SERP1, SSR3, QSCN6, NDUFB5, AP2M1, PSMD2, GLUL, RPL31, PP784, ARPC5, OCIAD1, HMGA1, CCNI, MAPKAPK2, PELO, ERBB2IP, SFRS12, RPS25, SERPINB6, TMEM14C, TTC1, STK10, MGAT1, MARCKS, C6orf62, VARS2, HSPA1A, NEU1, C6orf82, SRF, ELOVL5, EIF2AK1, KDELR2, C6orf111, CD164, STX7, PERP, GTF21, C7orf17, ACTB, SCRN1, YKT6, CTSB, WBSCR1, MDH2, HSPB1, YWHAG, ZNF655, FLJ22301, REPIN1, MGC8721, RAD23B, COBRA1, LY6E, SIAHBPI, NDUFB11, WDR13, DSIPI, CREB3, CLTA, NANS, GSN, HSPA5, EEF1A1, FBXW5, TMSB4X, EIF1AX, TIMP1, MAGED2, FLJ12525, PSMD10, BCAP31, IRAK1, EMD, NCOA4, EIF4EBP2, PSAP, DDIT4, SMP1, TRAPPC3, DDOST, NDUFB8, NOLC1, C1orf8, EIF3S10, PRDX3, HSPCA, SSRP1, TncRNA, POLD4, GSTP1, CCND1, INPPL1, AASDHPPT, DPAGT1, RNF26, RSU1, RAD52, FKBP4, ARHGAP21, MLF2, TPI1, PHC2, CBARA1, TUBA3, ATP5G2, RNF41, CTDSP2, RAB21, NAP1L1, PPIE, RAB35, RSN, SAP18, ZFP91, Ufm1, FLJ20277, ANKRD10, LRP10, C14orf119, ARF6, FLJ20580, RER1, PACS1L, C6orf49, PML, MDH1, HNRPDL, RPS23, AKAP9, ASAH1, OS-9, PKD1, DUT, HARS, NDUFS4, PITRM1, DDX17, SCAM-1, RPL6, GSPT1, UQCRC2, EIF3S4, SEPW1, NCK2, ANAPC7, ARPC2, AFAP, IFNAR1, UBE2G2, TLOC1, TFRC, RPL35A, VCP, BTF3, CSNK1A1, CANX, SQSTM1, SEC63, EIF3S2, LUC7L2, ANXA11, SSNA1, PDHA1, PIM3, PAI-RBP1, STXBP3, PMF1, RNH, MRPL16, FLJ20625, COPS7A, PRKAG1, LGALS3, PSMA3, SARS, CBWD1, BTBD14B, RANBP3, GAS5, DNCL2A, RAD1, RPL5, KIAA0663, GTL3, NMT1, KPNB1, HN1, MCL1, NDUFB7, HRIHFB2122, TUBB, SFRS10, LRPAP1, TOMM20, HSA9761, DDX46, NONO, PPP2R5D, VIM, TCEB1, WWP1, PPP1R8, LYPLA2, ATRX, H3F3A, RBM4, SRP14, STUB1, KRT8, TXNIP, EXOC7, ZNF258, HLA-C, NDUFA1, SEC22L1, B2M, CCND3, EIF5A, ITGB4BP, PPP4C, RPS7, SMARCB1, GRIM19, ANAPC11, C14orf166, TOMM22, MGC2941, PACS1, NDUFB9, CTSD, DNCI2, FDFT1, HNRPA1, RPL10A, PCBP2, RPS3, RPS12, RPS18, NET-5, SMAP, RPL13A, HSPC016, SSBP3, C9orf86, HTF9C, PGM3, C3F, NUP88, SREBF1, FL112953, MGC11257, FAM36A, C1QBP, HNRPAB, IDH3A, PSMC2, RBBP4, RBM8A, RIC8, PP1201, MRPS25, LONP, FBXL10, NDUFV2, PAICS, NPM1, ACP1, COX6A1, EEF1B2, EIF5, FKBP5, FTL, HSPE1, 1K, LAMR1, NDUFB4, NME2, NUMA1, RAC1, RNU65, RPL15, RPL18A, RPL19, RPL23A, RPL34, RPS3A, RPS8, RPS24, RPS26, SCD, CSDA, EIF3S5, G10, RPL14, PDCD6IP, TXNL2, ATP5L, NUDC, COPS8, C18orf10, 8D6A, PSARL, C2orf33, ORMDL1, PDE4DIP, CD2BP2, C1orf43, ATP2C1 and HCNGP disclosed in Korean Patent Publication No. 10-2008-0063156 disclosed by referring to an expressed sequence tag (EST) and SAGE human gene expression data from the public CGAP site (The Cancer Genome Project, //cgap.nci.nih.gov/) and data from the Affymetrix Human array set.

On the other hand, the determining of whether or not there is the copy number variation in the present invention may be performed by measuring the copy numbers of the target gene for copy number variation detection and the reference gene set of the present invention in a sample (preferably, a sample isolated from a human) and comparing the magnifications thereof.

Therefore, the present invention provides a method for determining whether there is copy number variation of a gene for copy number variation detection comprising the steps of:
(a) measuring the copy numbers of a target gene for copy number variation detection and a reference gene set in a sample;
(b) calculating a mean value of the copy number of each gene in the reference gene set; and
(c) calculating a value by dividing the copy number of the target gene for copy number variation detection by the mean value calculated in step (b),
wherein the value calculated in step (c) is compared with 2 to determine whether there is the copy number variation.

In the method, the mean value of step (b) is preferably an arithmetic mean value, and is an arithmetic mean value of all measured values of each gene in a reference gene set, and unmeasured or measurement errors during measurement may be excluded during the mean calculation.

In the determining of whether there is the copy number variation, it may be determined that there is copy number variation when the calculated value in step (c) is greater than 2, and it may be determined that there is no copy number variation when the calculated value is 2 or less.

In addition, when the reference gene set includes a gene located in an identical chromosome to the target gene for copy number variation detection, in order to prevent inaccurate discrimination due to polyploid chromosome (polysomy), a gene located in an identical chromosome to the target gene for copy number variation detection and a gene located in a non-identical chromosome are included, respectively. A value obtained by dividing the copy number of the target gene for copy number variation detection by the copy number of the gene located in the identical chromosome is 1.5 to 2.5. When the value obtained by dividing the copy number of the target gene for copy number variation detection by the copy number of the gene located in the non-identical chromosome is greater than 2, it may be determined that there is copy number variation, and when the value is 2 or less, it may be determined that there is no copy number variation.

Meanwhile, the present invention provides a primer and probe set composition for c-Met copy number variation (CNV) detection comprising at least one polynucleotide set as an active ingredient selected from the group consisting of i) a polynucleotide set for c-Met detection consisting of a primer of SEQ ID NO: 7, a primer of SEQ ID NO: 8, and a probe of SEQ ID NO: 1; and ii) a polynucleotide set consisting of a primer of SEQ ID NO: 9, a primer of SEQ ID NO: 10, and a probe of SEQ ID NO: 2; a polynucleotide set consisting of a primer of SEQ ID NO: 11, a primer of SEQ ID NO: 12, and a probe of SEQ ID NO; 3; a polynucleotide set consisting of a primer of SEQ 1D NO: 13, a primer of SEQ ID NO: 14, and a probe of SEQ ID NO: 4; a polynucleotide set consisting of a primer of SEQ ID NO: 15, a primer of SEQ ID NO: 16, and a probe of SEQ ID NO: 5; and a polynucleotide set consisting of a primer of SEQ ID NO: 17, a primer of SEQ ID NO: 18 and a probe of SEQ ID NO: 6, in order to detect copy number variation of a c-Met gene.

In the present invention, the ‘c-Met’ is a representative receptor tyrosine kinase (RTK) present on the cell surface, and binds to a ligand thereof, Hepatocyte Growth Factor/Scattering Factor (HGF/SF) to promote intracellular signaling and promote the growth of cells and is overexpressed in many types of cancer cells to be widely involved even in cancer occurrence, cancer metastasis, cancer cell migration, cancer cell invasion, and neovascularization. In addition, as the name of the ligand, the ‘c-Met’ is a representative protein in the early stage of cancer metastasis in which c-Met signaling through HGF/SF weakens a cell-cell contact of almost all types of epithelial tumors to cause scattering (Nat Rev Cancer. 2012) January 24; 12(2):89-103).

Therefore, the composition of the present invention capable of detecting the copy number variation (CNV) of the c-Met gene is not limited thereto, but may be used to provide information required for prognostic evaluation of cancer in which c-Met is expressed or prediction of the risk of mutation occurrence. In addition, the composition of the present invention may be used to provide information required for targeted therapy of cancer in which c-Met is expressed.

In the composition for copy number detection of the present invention, the ‘cancer’ means cancer in which c-Met is expressed. The cancer may be one or more selected from the group consisting of breast cancer, cervical cancer, cholangiocarcinoma, colorectal cancer, colon cancer, endometrial cancer, esophageal cancer, stomach cancer, head and neck cancer, kidney cancer, liver cancer, lung cancer, nasopharyngeal cancer, ovarian cancer, pancreatic cancer, gallbladder cancer, prostate cancer, thyroid cancer, osteosarcoma, rhabdomyosarcoma, synovial sarcoma, Kaposi sarcoma, leiomyosarcoma, malignant fibrous histiocytoma, fibrosarcoma, acute myeloid leukemia, adult T-cell leukemia, chronic myeloid leukemia, lymphoma, multiple myeloma, glioblastoma, astrocytoma, melanoma, mesothelioma, Wilms' tumor, and MiT tumor including clear cell sarcoma (CCS), alveolar soft part sarcoma (ASPS), and translocation-associated renal cell carcinoma, but it is not limited thereto.

In the present invention, the ‘primer’ means an oligonucleotide, and may serve as an initial point of synthesis under conditions in which synthesis of a primer extension product complementary to a nucleic acid strand (template) is induced, that is, in the presence of a polymer such as nucleotide and DNA polymerase, and in conditions of suitable temperature and pH. Preferably, the primer is deoxyribonucleotide and single-stranded. The primer used in the present invention may include naturally occurring dNMP (i.e., dAMP, dGMP, dCMP, and dTMP), modified nucleotides, or non-naturally occurring nucleotides. In addition, the primer may also include ribonucleotide.

The primer should be long enough to prime the synthesis of the extension product in the presence of a polymer. A suitable length of the primer is determined depending on a plurality of factors, such as a temperature, an application field, and a source of the primer, but is typically 15 to 30 nucleotides. Short primer molecules generally require a lower temperature to form a sufficiently stable hybrid complex with the template. The term ‘annealing’ or ‘priming’ refers to the apposition of an oligodeoxynucleotide or nucleic acid to a template nucleic acid, and the apposition allows the polymerase to polymerize the nucleotides to form a nucleic acid molecule complementary to the template nucleic acid or a portion thereof.

In the present invention, the ‘probe’ is designed as a kind of taqman probe used for quantitative PCR. Preferably, a fluorescent material (HEX, VIC, FAM dye) was attached to the probe, and BHQ1 may be used as a quencher on 3′ ends of all probes. A TaqMan probe is generally an oligonucleotide tagged with a fluorescent material at a 5′ end and a quencher at a 3′ end. The TaqMan probe hybridizes specifically to template DNA in an annealing step, but does not emit fluorescence even when light is given because there is a quencher at the 3′ end of the probe. However, when the TaqMan probe hybridized to the template is decomposed by 5′→3′ exonuclease activity of Taq DNA polymerase in an extension step, the next process, fluorescence according to PCR reaction is quantitatively emitted by a principle that the fluorescence material is separated from the probe to release the suppression by the quencher and emit the fluorescence.

In the present invention, the probe may be bound with the fluorescent material, and more preferably, may be bound with hexachlorofluorescein (HEX), fluorescein amidite (FAM), and EverGreen fluorescent dyes.

Specifically, since a form in which the FAM and HEX fluorescent dyes (fluorescent materials) or EvaGreen fluorescent dye was bound to the probe was used, this process may be performed by measuring the fluorescence therefor. Such a process may be performed by a commercial detection device (e.g., a droplet reader from biorad Co., Ltd.), and the analysis may be automatically completed by detecting a droplet fluorescence signal of each sample in the device and counting the number of positive and negative droplets.

At this time, a probe added to the PCR reaction solution and a probe added to the standard PCR reaction solution for detection may be bound to different fluorescent materials, respectively.

The present invention provides a kit for detecting c-Met gene copy number variation (CNV) comprising the primer and probe set of the present invention.

The present invention provides a kit for detecting c-Met copy number variation (CNV) comprising at least one polynucleotide set as an active ingredient selected from the group consisting of i) a polynucleotide set for c-Met detection consisting of a primer of SEQ ID NO: 7, a primer of SEQ ID NO: 8, and a probe of SEQ ID NO: 1; and ii) a polynucleotide set consisting of a primer of SEQ ID NO: 9, a primer of SEQ ID NO: 10, and a probe of SEQ ID NO: 2; a polynucleotide set consisting of a primer of SEQ ID NO: 11, a primer of SEQ ID NO: 12, and a probe of SEQ ID NO: 3; a polynucleotide set consisting of a primer of SEQ ID NO: 13, a primer of SEQ ID NO: 14, and a probe of SEQ ID NO: 4; a polynucleotide set consisting of a primer of SEQ ID NO: 15, a primer of SEQ ID NO: 16, and a probe of SEQ ID NO: 5; and a polynucleotide set consisting of a primer of SEQ ID NO: 17, a primer of SEQ ID NO: 18 and a probe of SEQ ID NO: 6.

The kit of the present invention may be preferably used for CNV detection of the c-Met gene by PCR reaction using the primer/probe set of the present invention. The kit of the present invention may further include tools and/or reagents known in the art to be used for PCR or detection thereof. The kit of the present invention may further include, if necessary, instruction data disclosing a tube to be used for mixing respective components, a well plate, and a using method.

In addition, the kit of the present invention may be a research use only (RUO) or in vitro diagnostics (IVD) kit. The IVD kit also includes an in vitro companion diagnostics (IVD-CDx) kit.

The kit of the present invention is used for a quantitative PCR. (qPCR) or droplet digital PCR method.

The template applicable to the kit of the present invention requires CNV detection of the c-Met gene, and may be used without limitation as long as the PCR reaction can be performed. Preferably, cell-free DNA (cfDNA) isolated from the blood, genomic DNA (gDNA) derived from circulating tumor cells (CTCs), DNA isolated from formalin fixed paraffin embedded (FFPE) tissue, or complementary DNA (cDNA) synthesized by reverse transcription reaction from RNA derived from the tissue is used as the template.

The cfDNA circulating in the human plasma has been studied in a variety of physiological and pathological conditions, such as inflammatory disorders, oxidative stress, and malignancies. Although an exact mechanism associated with the bloodstream release of cfDNA is uncertain, apoptosis, necrocytosis, and active release from cells seem to work synthetically. cfDNA circulating through blood vessels is a potentially useful biomarker. DNA levels and fragmentation patterns represent interesting possibilities for diagnostic and prognostic purposes. In particular, since the cfDNA may be easily detected in the blood of a patient, there is usefulness in that a biomarker may be detected simply and quickly without significantly impairing the quality of life.

Furthermore, the kit of the present invention may use as DNA isolated from circulating tumor cells (CTCs) isolated from the blood or cDNA synthesized by reverse transcription from the CTC-derived RNA as the template. The CTCs are tumor cells found in the peripheral blood of malignant tumor patients. Since the CTC plays an important role in cancer metastasis, the CTC is considered very important in research, diagnosis, and the like of cancer, but since the number of CTCs in the peripheral blood is very rare, there is a need for a detection system that requires sensitivity enough to detect tens or less of tumor cells mixed with millions or more of normal blood cells.

The tissue obtained from the patient after the biopsy is usually immobilized with formalin (formaldehyde) or the like. The immobilized biological sample is generally dehydrated and embedded in a solid support such as paraffin, and the prepared sample is referred to as a FFPE sample. Since nucleic acids, particularly DNA, on the FFPE sample exist in immobilized cells and are fragmented or cross-linked by formalin, it is necessary to remove paraffin and lyse the immobilized cells to elute nucleic acids including DNA in the cells.

In the present invention, the term ‘paraffin’ comprehensively refers to an embedded medium of a biological sample used in all analysis including morphological, immunohistochemical and enzymatic histochemical analysis. That is, the paraffin in the present invention may be a petroleum-based paraffin wax monomer, and may include all other materials that may be added for the purpose of improving the quality of the embedded medium using the petroleum-based paraffin wax as a base material. Here, the petroleum-based paraffin wax refers to a mixture of hydrocarbons that are derived from petroleum and solid at room temperature.

In general, an FFPE-treated specimen of a cancer patient is cut to a thickness of 5 to 10 μm with a rotary microtome, and then nucleic acids containing DNA may be isolated using a commercially available nucleic acid isolation kit for FFPE or a device using the same. The kit/device for isolating the nucleic acids from the FFPE may include, for example, a tissue preparation system from Siemens Co., Ltd. and related reagents (VERSANT tissue preparation reagents).

The nucleic acid isolated from the sample of the present invention is preferably genomic DNA, more preferably genomic DNA estimated to have CNV.

The composition or kit of the present invention may be preferably used for CNV detection of the c-Met gene by an automated or semi-automated method. The automation may mean that adding of a sample; repositioning or moving of a substrate (e.g., tube, plate) on which extraction, separation, and reaction have been completed; addition and replenishment of reagents and buffers to a stock; and all or most of processes except for the maintenance of equipment are performed by a means (e.g., robots) other than humans.

For reference, for the above-mentioned nucleotide operation, methods known in the art may be referred to.

The ‘sample’ of the present invention includes blood and other liquid samples of biological origin, biopsy samples, solid tissue samples such as tissue culture, or cells derived therefrom. More specifically, for example, but not limited thereto, the sample may include tissue, extract, cell lysate, whole blood, plasma, serum, saliva, ocular fluid, cerebrospinal fluid, sweat, urine, milk, ascites fluid, synovial fluid, peritoneal fluid, etc. The sample may be obtained from animals, preferably mammals, and most preferably humans. In addition, the sample may be pretreated before use for detection. For example, the pretreating may include filtration, distillation, extraction, concentration, inactivation of interfering components, addition of reagents, and the like. Preferably, the sample may be a circulating tumor cell (CTC), blood, formalin fixed paraffin embedded (FFPE), cell-free DNA (cfDNA), fresh sample, etc. obtained from a patient, and most preferably, the sample may be blood or formalin fixed paraffin embedded (FFPE) obtained from the patient. As described above, genomic DNA or DNA estimated to have CNV may be isolated from the sample to be used for CNV detection of the c-Met gene.

As described above, the detection method of the present invention may detect the CNV of the c-Met gene by a quantitative PCR (qPCR) or digital PCR method.

Further, the present invention provides uses of at least one polynucleotide set from the group consisting of
i) a polynucleotide set for c-Met detection consisting of a primer of SEQ ID NO: 7, a primer of SEQ ID NO: 8, and a probe of SEQ ID NO: 1; and
ii) a polynucleotide set consisting of a primer of SEQ ID NO: 9, a primer of SEQ ID NO: 10, and a probe of SEQ ID NO: 2; a polynucleotide set consisting of a primer of SEQ ID NO: 11, a primer of SEQ ID NO: 12, and a probe of SEQ ID NO: 3; a polynucleotide set consisting of a primer of SEQ ID NO: 13, a primer of SEQ ID NO: 14, and a probe of SEQ ID NO: 4; a polynucleotide set consisting of a primer of SEQ ID NO: 15, a primer of SEQ ID NO: 16, and a probe of SEQ ID NO: 5; and a polynucleotide set consisting of a primer of SEQ ID NO: 17, a primer of SEQ ID NO: 18 and a probe of SEQ ID NO: 6, in order to prepare a primer and probe set formulation for copy number variation detection of a c-Met gene.

Further, the present invention provides a method for detecting copy number variation (CNV) of a c-Met gene comprising:
(a) administering, to a target gene for CNV detection in a sample, at least one polynucleotide set from the group consisting of
i) a polynucleotide set for c-Met detection consisting of a primer of SEQ ID NO: 7, a primer of SEQ ID NO: 8, and a probe of SEQ ID NO: 1; and
ii) a polynucleotide set consisting of a primer of SEQ ID NO: 9, a primer of SEQ ID NO: 10, and a probe of SEQ ID NO: 2; a polynucleotide set consisting of a primer of SEQ ID NO: 11, a primer of SEQ ID NO: 12, and a probe of SEQ ID NO: 3; a polynucleotide set consisting of a primer of SEQ ID NO: 13, a primer of SEQ ID NO: 14, and a probe of SEQ ID NO: 4; a polynucleotide set consisting of a primer of SEQ ID NO: 15, a primer of SEQ ID NO: 16, and a probe of SEQ ID NO: 5; and a polynucleotide set consisting of a primer of SEQ ID NO: 17, a primer of SEQ ID NO: 18 and a probe of SEQ ID NO: 6;
(b) measuring the copy number of a reference gene set of the present invention;
(c) calculating a mean value of the copy numbers of each gene in the reference gene set;
(d) calculating a value by dividing the copy number of the target gene for copy number variation detection by the mean value calculated in step (c); and
(e) comparing the value calculated in step (d) with 2 and determining that the copy number variation exists when the value is greater than 2.

The term “comprising” used herein is used in the same meaning as “including” or “characterized by”, and does not exclude additional ingredients or steps of the method which are not specifically mentioned in the composition or the method according to the present invention. The term “consisting of” means excluding additional elements, steps or ingredients, etc., unless otherwise described. The term “essentially consisting of” means including materials or steps which do not substantially affect basic properties thereof in addition to the described materials or steps within the range of the composition or the method.

### Advantageous Effects

Accordingly, the present invention provides a primer and probe set composition for detecting CNV of a c-Met gene, and a kit including the same. According to the present invention, since the method may predict a prognosis in a c-Met-expressing cancer patient or a risk of mutation occurrence and may provide information required for targeted therapy of cancer, the method may be effectively used to provide clues for future treatment directions, as well as to determine a need to administer an anticancer drug, and to monitor the metastasis or recurrence of cancer.

### DESCRIPTION OF DRAWINGS

FIG. 1 is a result of re-establishing a reference gene for CNV detection of a c-Met gene, which is a result of grouping based on an amplitude value for each reference and then performing the CNV detection for Met target exons 2 and 15 (c-Met exon2(left), exon15 (right)).

FIG. 2 is a result of calculating a CNV value for a value calculated as a mean for each reference group after setting a threshold and analyzing the copy number.

FIGS. 3 to 5 are results of performing pre-analytical validation with respect to panels (referred to as MMX1, MMX2 or MMX3) with detected CNV among panels known to have c-Met CNV and confirming the presence or absence of an interference reaction through cross-reaction.

FIGS. 6A to 6D are results illustrating the copy numbers and CNV values according to an interference reaction test results for each panel, wherein FIG. 6A is a result illustrating the copy number of a panel MMX1, FIG. 6B is a result illustrating the copy number of a panel MMX2, FIG. 6C is a result illustrating the copy number of a panel MMX3, and FIG. 6D is a result illustrating the CNV values of the panels.

FIGS. 7A and 7B are results of measuring LOB values by confirming CNV values after repetitively experimenting an interference reaction between a reference material and a human gDNA sample, wherein FIG. 7A is a result of 10 repeated experiments and FIG. 7B is a result illustrating LOB values according to 20 FFPE samples.

FIG. 8 is a result of repeating an LOD experiment to determine the minimum concentration detected by serial dilution of input DNA amounts of human gDNA and reference materials.

FIG. 9 is a result of confirming whether CNV can be detected in cfDNA in the blood, which is a result of confirming c-Met CNV detection in DNA cut for each panel.

FIG. 10 is a result of confirming whether CNV can be detected in cfDNA in the blood, which is a result illustrating c-Met copy numbers and CNV values in DNA cut for each panel.

FIGS. 11A and 11B are results of confirming the minimum detectable concentration of CNV in a cell line known to have CNV, wherein FIG. 1I A is a result confirmed after mixing human gDNA to at least 20%, and FIG. 11B is a result confirmed after mixing human gDNA to at least 1%.

### MODES FOR THE INVENTION

Hereinafter, preferred Examples will be proposed in order to help in understanding of the present invention. However, the following Examples are just provided to more easily understand the present invention and the contents of the present invention are not limited by Examples.

### Example 1: Construction of Data Set for Housekeeping Genes

In order to search for housekeeping genes (HKG) that were expressed at similar levels in various tissues of the human body, the present invention first referred to expressed sequence tag (EST) and SAGE human gene expression data from a public CGAP site The Cancer Genome Project, //cgap.nci.nih.gov/) and microarray gene expression data from GeneExpress Oncology Datasuite™ from Gene Logic Co., Ltd. fabricated based on an Affymetrix Human Genome U133(HG-U133) array set so as to construct a data set. The selected candidate endogenous reference gene is characterized to be a gene selected from the group consisting of Accession No. Hs 120 (PRDX6), Accession No. Hs 142(SULT1A1), Accession No. Hs 202(BZRP), Accession No. Hs 429(ATP5G3), Accession No. Hs 695(CSTB), Accession No. Hs 808(HNRPF), Accession No. Hs 861(MAPK3), Accession No. Hs 1063(SNRPC), Accession No. Hs I103(TGFB1), Accession No. Hs 2430(TCFL1), Accession No. Hs 2533(ALDH9A1), Accession No. Hs 2795(LDHA), Accession No. Hs 2853(PCBP1), Accession No. Hs 3100(KARS), Accession No. Hs 3254(MRPL23), Accession No. Hs 3353(G3BP), Accession No. Hs 3416(ADFP), Accession No. Hs 439(STOML2), Accession No. Hs 3530(FUSIP1), Accession No. Hs 3989(PLXNB2), Accession No. Hs 4055(KLF6), Accession No. Hs 4742(GPAA1), Accession No. Hs 4747(DKC1), Accession No. Hs 4766(FAM32A), Accession No. Hs 4859(CCNL1), Accession No. Hs 4997(RBM23), Accession No. Hs 4998(TMOD3), Accession No. Hs 5062(TM4SF8), Accession No. Hs 5086(MGC10433), Accession No. Hs 5120(DNCL1), Accession No. Hs 5158(ILK), Accession No. Hs 5245(FLJ20643), Accession No. Hs 5258(MAGED1), Accession No. Hs 5268(ZDHHC4), Accession No. Hs 5298(ADIPOR1), Accession No. Hs 5308(UBA52), Accession No. Hs 5324(C2orf25), Accession No. Hs 5345(RNPEPL1), Accession No. Hs 5662(GNB2L1), Accession No. Hs 5710(CREG1), Accession No. Hs 5719(CNAP1), Accession No. Hs 5912(FBXO7), Accession No. Hs 5947(RAB8A), Accession No. Hs 6396(JTB), Accession No. Hs 6454(RGS191P1), Accession No. Hs 6459(GPR172A), Accession No. Hs 6551(ATP6AP1), Accession No. Hs 6891(SFRS6), Accession No. Hs 7101(ANAPC5), Accession No. Hs 7236(NOSIP), Accession No. Hs 7476(ATP6V0B), Accession No. Hs 7527(DKFZP566E144), Accession No. Hs 7744(NDUFV1), Accession No. Hs 7753(CALU), Accession No. Hs 7768(FIBP), Accession No. Hs 7862(PNRC2), Accession No. Hs 7910(RYBP), Accession No. Hs 7917(HIG1), Accession No. Hs 8102(RPS20), Accession No. Hs 8372(UQCR), Accession No. Hs 8737(WDR6), Accession No. Hs 8752(TMEM4), Accession No. Hs 8765(DDX42), Accession No. Hs 8859(CANT1), Accession No. Hs 8867(CYR61), Accession No. Hs 9003(FLJ13868), Accession No. Hs 9015(MGC52000), Accession No. Hs 9043(C14orf120), Accession No. Hs 9234(NIFIE14), Accession No. Hs 9235(NME4), Accession No. Hs 9527(C2orf28), Accession No. Hs 9534(SEC1 L1), Accession No. Hs 9573(ABCF1), Accession No. Hs 9589(UBQLN1), Accession No. Hs 9788(NDFIP1), Accession No. Hs 9825(CGI-128), Accession No. Hs 9857(DCXR), Accession No. Hs 10326(COPE), Accession No. Hs 10842(RAN), Accession No. Hs 10848(BMS1L), Accession No. Hs 11125(SPCS1), Accession No. Hs 11184(UBE2R2), Accession No. Hs 11223(IDH1), Accession No. Hs 11355(TMPO), Accession No. Hs 11463(UMP-CMPK), Accession No. Hs 12013(ABCE1), Accession No. Hs 12084(TUFM), Accession No. Hs 12102(SNX3), Accession No. Hs 12107(BC-2), Accession No. Hs 12109(WDR39), Accession No. Hs 12144(KIAA1033), Accession No. Hs 12152(SRPRB), Accession No. Hs 12272(BECN1), Accession No. Hs 12341(ADAR), Accession No. Hs 12457(NUP133), Accession No. Hs 12865(NSFL1C), Accession No. Hs 13662(MGC5508), Accession No. Hs 14317(NOLA3), Accession No. Hs 14333(FLJ10349), Accession No. Hs 14745(C10orf9), Accession No. Hs 14839(POLR2G), Accession No. Hs 14846(SLC7A1), Accession No. Hs 14894(TGOLN2), Accession No. Hs 15277(C16orf33), Accession No. Hs 15591(COPS6), Accession No. Hs 15738(RAB7), Accession No. Hs 16059(HSPC009), Accession No. Hs 16130(E2-230K), Accession No. Hs 16349(KIAA0431), Accession No. Hs 17118(FLJ11730), Accession No. Hs 17250(MGC4767), Accession No. Hs 17680(FUCA2), Accession No. Hs 17731(FLJ12892), Accession No. Hs 17883(PPM1 G), Accession No. Hs 18069(LGMN), Accession No. Hs 18128(C20orf44), Accession No. Hs 18349(MRPL15), Accession No. Hs 19673(MAF1), Accession No. Hs 20013(P29), Accession No. Hs 20107(KNS2), Accession No. Hs 20157(CDK5RAP3), Accession No. Hs 20521(HRMT1L2), Accession No. Hs 20529(LOC127262), Accession No. Hs 20573(IGF1R), Accession No. Hs 20716(TIMM17A), Accession No. Hs 22393(DENR), Accession No. Hs 22543(UBE3A), Accession No. Hs 22546(CYBASC3), Accession No. Hs 22616(KIAA0664), Accession No. Hs 23033(LOC92912), Accession No. Hs 23111(FARSLA), Accession No. Hs 23978(SAFB), Accession No. Hs 24301(POLR2E), Accession No. Hs 24379(TRAPPC1), Accession No. Hs 24601(FBLN1), Accession No. Hs 24950(RGS5), Accession No. Hs 25155(NET1), Accession No. Hs 25450(SLC29A1), Accession No. Hs 25723(MTVR1), Accession No. Hs 26010(PFKP), Accession No. Hs 26023(FOXJ3), Accession No. Hs 26136(MGC14156), Accession No. Hs 26232(MAN2C1), Accession No. Hs 26403(GSTZ1), Accession No. Hs 26518(TM4SF7), Accession No. Hs 27222(NOLA2), Accession No. Hs 28491(SAT), Accession No. Hs 28914(APRT), Accession No. Hs 29203(GBL), Accession No. Hs 29665(CLSTN1), Accession No. Hs 30011(MGC2963), Accession No. Hs 30026(HSPC182), Accession No. Hs 30345(TRAP1), Accession No. Hs 30954(PMVK), Accession No. Hs 31053(CKAP1), Accession No. Hs 31334(C20orf14), Accession No. Hs 31387(DKFZP564J0123), Accession No. Hs 34045(CDCA4), Accession No. Hs 34576(TAX1BP1), Accession No. Hs 34906(BLOC1S2), Accession No. Hs 35052(TEGT), Accession No. Hs 35828(MARK3), Accession No. Hs 36587(PPP1R7), Accession No. Hs 36927(HSPH1), Accession No. Hs 37616(STRA13). Accession No. Hs 37916(DPP7), Accession No. Hs 42806(Cab45), Accession No. Hs 43297(MTPN), Accession No. Hs 47062(POLR2I), Accession No. Hs 50098(NDUFA4), Accession No. Hs 50308(HIP2), Accession No. Hs 50425(TEBP), Accession No. Hs 53066(HSPBP1), Accession No. Hs 54277(FAM50A), Accession No. Hs 54457(CD81), Accession No. Hs 54642(MAT2B), Accession No. Hs 54649(RY1), Accession No. Hs 55682(EIF3S7), Accession No. Hs 55847(MRPL51), Accession No. Hs 58488(CTNNAL1), Accession No. Hs 58992(SMC4L1), Accession No. Hs 59486(HSDL2), Accession No. Hs 61812(PTPN12), Accession No. Hs 65234(DDX27), Accession No. Hs 65238(RNF40), Accession No. Hs 66048(BPY21P1), Accession No. Hs 66915(C22orf16), Accession No. Hs 68714(SFRS1), Accession No. Hs 9293(HEXB), Accession No. Hs 69554(RNF126), Accession No. Hs 69855(UNR), Accession No. Hs 71465(SQLE), Accession No. Hs 71787(MRPS7), Accession No. Hs 73527(CSNK2B), Accession No. Hs 73722(APEX1), Accession No. Hs 73799(GNA13), Accession No. Hs 73965(SFRS2), Accession No. Hs 74047(ETFB), Accession No. Hs 74050(FVT1), Accession No. Hs 74137(TMP21), Accession No. Hs 74375(DVL1), Accession No. Hs 74405(YWHAQ), Accession No. Hs 74471(GJA1), Accession No. Hs 74563(OAZ2), Accession No. Hs 74564(SSR2), Accession No. Hs 74576(GDI1), Accession No. Hs 75056(TIMM13), Accession No. Hs 75061(MARCKSL1), Accession No. Hs 75066(TSN), Accession No. Hs 75087(FASTK), Accession No. Hs 75117(ILF2), Accession No. Hs 75133(TFAM), Accession No. Hs 75139(ARFIP2), Accession No. Hs 75189(DAP), Accession No. Hs 75227(NDUFA9), Accession No. Hs 75243(BRD2), Accession No. Hs 75249(ARL61P), Accession No. Hs 75254(IRF3), Accession No. Hs 75318(TUBA1), Accession No. Hs 75348(PSME1), Accession No. Hs 75438(QDPR), Accession No. Hs 75527(ADSL), Accession No. Hs 75724(COPB2), Accession No. Hs 75798(C20orf111), Accession No. Hs 75841(C12orf8), Accession No. Hs 75890(MBTPS1), Accession No. Hs 75914(RNP24), Accession No. Hs 76111(DAG1), Accession No. Hs 76394(ECHS1), Accession No. Hs 76480(UBL4), Accession No. Hs 76662(ZDHHC16), Accession No. Hs 76686(GPX1), Accession No. Hs 76847(GANAB), Accession No. Hs 77060(PSMB6), Accession No. Hs 77269(GNAI2), Accession No. Hs 77313(CDK10), Accession No. Hs 77422(PLP2), Accession No. Hs 77558(HMGN3), Accession No. Hs 77578(USP9X), Accession No. Hs 77793(CSK), Accession No. Hs 77897(SF3A3), Accession No. Hs 77961(HLA-B), Accession No. Hs 77978(DKFZp76112123), Accession No. Hs 78466(PSMD8), Accession No. Hs 78601(UROD), Accession No. Hs 78771(PGK1), Accession No. Hs 78880(ILVBL), Accession No. Hs 78888(DBI), Accession No. Hs 78989(ADH5), Accession No. Hs 79064(DHPS), Accession No. Hs 79081(PPP1CC), Accession No. Hs 79088(RCN2), Accession No. Hs 79101(CCNG1), Accession No. Hs 79110(NCL), Accession No. Hs 79322(QARS), Accession No. Hs 79335(SMARCD1), Accession No. Hs 79387(PSMC5), Accession No. Hs 79402(POLR2C), Accession No. Hs 79411(RPA2), Accession No. Hs 79625(C20orf149), Accession No. Hs 80545(RPL37), Accession No. Hs 80919(SYPL), Accession No. Hs 80986(ATP5G1), Accession No. Hs 81328(NFKBIA), Accession No. Hs 81424(SUMO1), Accession No. Hs 81848(RAD21), Accession No. Hs 81964(SEC24C), Accession No. Hs 82201(CSNK2A2), Accession No. Hs 82327(GSS), Accession No. Hs 82719(MGC21416), Accession No. Hs 82793(PSMB3), Accession No. Hs 82887(PPP1R11), Accession No. Hs 82890(DAD1), Accession No. Hs 82916(CCT6A), Accession No. Hs 82927(AMPD2), Accession No. Hs 83190(FASN), Accession No. Hs 83347(AAMP), Accession No. Hs 83383(PRDX4), Accession No. Hs 83734(STX4A), Accession No. Hs 83753(SNRPB), Accession No. Hs 83765(DHFR), Accession No. Hs 83916(NDUFA5), Accession No. Hs 84359(GABARAP), Accession No. Hs 84753(FLJ12442), Accession No. Hs 85155(ZFP36L1), Accession No. Hs 85769(ERBP), Accession No. Hs 85962(DERPC), Accession No. Hs 86131(FADD), Accession No. Hs 87752(MSN), Accession No. Hs 89545(PSMB4), Accession No. Hs 89643(TKT), Accession No. Hs 89649(EPHX1), Accession No. Hs 89781(UBTF), Accession No. Hs 89864(SKIV2L), Accession No. Hs 90061(PGRMC1), Accession No. Hs 90093(HSPA4), Accession No. Hs 90107(ADRM1), Accession No. Hs 90443(NDUFS8), Accession No. Hs 91142(KHSRP), Accession No. Hs 91531(MLLT6), Accession No. Hs 93659(ERP70), Accession No. Hs 93832(LOC54499), Accession No. Hs 95577(CDK4), Accession No. Hs 96530(COX11), Accession No. Hs 96852(FLJ21128), Accession No. Hs 96996(HNRPA0), Accession No. Hs 97616(SH3GL1), Accession No. Hs 97887(RCN1), Accession No. Hs 98751(FUBP3), Accession No. Hs 98791(ACTR1B), Accession No. Hs 102696(MCTS1), Accession No. Hs 102798(PSMA1), Accession No. Hs 103561(ARL61P4), Accession No. Hs 103834(MGC5576), Accession No. Hs 104839(TIMP2), Accession No. Hs 105547(NPDC1), Accession No. Hs 106185(RALGDS), Accession No. Hs 106876(ATP6V0D1), Accession No. Hs 106909(ANAPC13), Accession No. Hs 107003(CCNB11P1), Accession No. Hs 107101(FLJ31031), Accession No. Hs 107387(C7orf20), Accession No. Hs 107393(C3orf4), Accession No. Hs 108029(SH3BGRL), Accession No. Hs 108080(CSRP1), Accession No. Hs 108371(E2F4), Accession No. Hs 108408(APH-1A), Accession No. Hs 108957(RPS27L), Accession No. Hs 108969(PTD08), Accession No. Hs 109051(SH3BGRL3), Accession No. Hs 109052(C14orf2), Accession No. Hs 109672(SIAT7F), Accession No. Hs 109798(C6orf48), Accession No. Hs 110695(SF3B5), Accession No. Hs 110849(ESRRA), Accession No. Hs 111286(MRPS11), Accession No. Hs 111577(ITM2C), Accession No. Hs 111801(ARS2), Accession No. Hs 112058(SIVA), Accession No. Hs 112318(TOMM7), Accession No. Hs 112955(NUDT5), Accession No. Hs 114033(SSR1), Accession No. Hs 114286(CD9), Accession No. Hs 114412(TXNL1), Accession No. Hs 115474(RFC3), Accession No. Hs 115792(EXOSC7), Accession No. Hs 116448(GLS), Accession No. Hs 117176(PABPN1), Accession No. Hs 117715(ST5), Accession No. Hs 118110(BST2), Accession No. Hs 118400(FSCN1), Accession No. Hs I18463(PNPLA2), Accession No. Hs 118638(NME1), Accession No. Hs 118722(FUT8), Accession No. Hs 118964(p66alpha), Accession No. Hs 118983(GSDMDC1), Accession No. Hs 19177(ARF3), Accession No. Hs 119192(H2AFZ), Accession No. Hs 119251(UQCRC1), Accession No. Hs 119591(AP2S1), Accession No. Hs 119598(RPL3), Accession No. Hs 120323(DNAPTP6), Accession No. Hs 121088(NUP153), Accession No. Hs 121549(CDIPT), Accession No. Hs 122363(WIPI-2), Accession No. Hs 122523(SND1), Accession No. Hs 124126(ARPC1A), Accession No. Hs 124147(FBXL11), Accession No. Hs 124246(C10orf119), Accession No. Hs 124366(BBX), Accession No. Hs 125113(CCT8), Accession No. Hs 125867(EVL), Accession No. Hs 125898(GNAS), Accession No. Hs 126497(AEBP2), Accession No. Hs 126774(RAMP), Accession No. Hs 126938(NAPA), Accession No. Hs 127092(DHX38), Accession No. Hs 127249(EAP30), Accession No. Hs 127386(MAMDC2), Accession No. Hs 127764(RAB5C), Accession No. Hs 128065(CTSC), Accession No. Hs 128199(SEPT11), Accession No. Hs 128548(WDR1), Accession No. Hs 129634(CINP), Accession No. Hs 129673(EIF4A1), Accession No. Hs 130031(TRIO), Accession No. Hs 130098(DDX23), Accession No. Hs 130293(CROP), Accession No. Hs 130413(TM9SF2), Accession No. Hs 131226(BNIP3L), Accession No. Hs 132497(PRNPIP), Accession No. Hs 132513 HSD17B12), Accession No. Hs 133892(TPM1), Accession No. Hs 134074(SLC35E1), Accession No. Hs 134688(PSMD13), Accession No. Hs 135406(CEBPZ), Accession No. Hs 136905(UREB1), Accession No. Hs 136947(RALY), Accession No. Hs 137510(NCOR2), Accession No. Hs 138860(ARHGAP1), Accession No. Hs 139896(MAEA), Accession No. Hs 140452(M6PRBP1), Accession No. Hs 142442(HP1-BP74), Accession No. Hs 143187(DDX49), Accession No. Hs 143766(DRPLA), Accession No. Hs 143873(S100A10), Accession No. Hs 144058(EBSP), Accession No. Hs 144468(MGC3234), Accession No. Hs 144835(EEF1G), Accession No. Hs 144868(VTI1B), Accession No. His 144941(MUF1), Accession No. Hs 144949(ZNF313), Accession No. Hs 144980(SCAMP4), Accession No. Hs 145049(PLEKHM2), Accession No. Hs 145442(MAP2K1), Accession No. Hs 145575(UBL3), Accession No. Hs 146070(TPM3), Accession No. Hs 146393(HERPUD1), Accession No. Hs 146602(QP-C), Accession No. Hs 146804(SPIN), Accession No. Hs 146806(CUL1), Accession No. Hs 147433(PCNA), Accession No. Hs 148078(RBAF600), Accession No. Hs 148272(CCM2), Accession No. Hs 148330(ARF4), Accession No. Hs 148340(PTPRG), Accession No. Hs 148670(RHOBTB1), Accession No. Hs 149004(FBXO31), Accession No. Hs 149957(RPS6KA1), Accession No. Hs 149983(PEX14), Accession No. Hs 150107(BIRC6), Accession No. Hs 150540(BC002942), Accession No. Hs 150580(SUI1), Accession No. Hs 150837(TXNDC5), Accession No. Hs 151134(OXA1L), Accession No. Hs 151220(KIAA0992), Accession No. Hs 151413(GMFB), Accession No. Hs 151787(U5-116KD), Accession No. Hs 152536(p44S10), Accession No. Hs 153177(RPS28), Accession No. Hs 154023(TXNDC4), Accession No. Hs 154073(SLC35B1), Accession No. Hs 155165(ZFPL1), Accession No. Hs 155218(HNRPUL1), Accession No. Hs 155396(NFE2L2), Accession No. Hs 155829(KIAA0676), Accession No. Hs 156171(PSMC6), Accession No. Hs 156367(RPS29), Accession No. Hs 156667(KIAA1536), Accession No. Hs 157160(MRPS34), Accession No. Hs 157351(PTD004), Accession No. Hs 157379(H2AFV), Accession No. Hs 157394(HAGH), Accession No. Hs 159014(PRPF4B), Accession No. Hs 159118(AMD1), Accession No. Hs 159130(RAF1), Accession No. Hs 159161(ARHGDIA), Accession No. Hs 159699(FBXO21), Accession No. Hs 159799(THRAP2), Accession No. Hs 160958(CDC37), Accession No. Hs 161357(PDHB), Accession No. Hs 162032(HBP1), Accession No. Hs 162233(CHD4), Accession No. Hs 162877(PACSIN2), Accession No. Hs 163645(MOCS2), Accession No. Hs 163776(UBE211), Accession No. Hs 163893(PICALM), Accession No. Hs 165195(VAPA), Accession No. Hs 166011(CTNND1), Accession No. Hs 166204(PHF1), Accession No. Hs 166463(HNRPU), Accession No. Hs 166924(SEC13L1), Accession No. Hs 166975(SFRS5), Accession No. Hs 167535(SRP54), Accession No. Hs 168073(TRPC4AP), Accession No. Hs 168799(METTL3), Accession No. Hs 169611(DIABLO), Accession No. Hs 169718(CNN2), Accession No. Hs 170107(UQCRFS1), Accession No. Hs 170131(NF1C), Accession No. Hs 170553(CNOT7), Accession No. Hs 170622(CFL1), Accession No. Hs 171626(SKP1A), Accession No. Hs 172550(PTBP1), Accession No. Hs 172755(BRP44L), Accession No. Hs 172928(COL1A1), Accession No. Hs 173024(NYREN18), Accession No. Hs 173162(NOC4), Accession No. Hs 173381(DPYSL2), Accession No. Hs 173464(FKBP8), Accession No. Hs 173611(NDUFS2), Accession No. Hs 173705(LOC401152), Accession No. Hs 173724(CKB), Accession No. Hs 174050(EDF1), Accession No. Hs 174195(IFITM2), Accession No. Hs 175473(AK1), Accession No. Hs 175955(YT521), Accession No. Hs 177530(ATP5E), Accession No. Hs 177766(PARP1), Accession No. Hs 178551(RPL8), Accession No. Hs 178728(MBD3), Accession No. Hs 179986(FLOT1), Accession No. Hs 180141(CFL2), Accession No. Hs 180312(MRPS16), Accession No. Hs 180414(HSPA8), Accession No. Hs 180877(H3F3B), Accession No. Hs 180903(384D8-2), Accession No. Hs 180909(PRDX1), Accession No. Hs 180933(CXXC1), Accession No. Hs 181046(DUSP3), Accession No. Hs 181112(MED4), Accession No. Hs 181163(HMGN2), Accession No. Hs 181244(HLA-A), Accession No. Hs 181368(PRPF8), Accession No. Hs 181444(TMEM9), Accession No. Hs 182255(NHP2L1), Accession No. Hs 182626(C22orf5), Accession No. Hs 182885(SLC35B2), Accession No. Hs 183684(EIF4G2), Accession No. Hs 183706(ADD1), Accession No. Hs 183800(RANGAP1), Accession No. Hs 183850(DCTD), Accession No. Hs 183994(PPP1CA), Accession No. Hs 184062(C20orf24), Accession No. Hs 184211(PMPCB), Accession No. Hs 184233(HSPA9B), Accession No. Hs 184492(ELAVL1), Accession No. Hs 185172(GNB2), Accession No. Hs 185597(SPG7), Accession No. Hs 187199(MALAT1), Accession No. Hs 187635(RPS15A), Accession No. Hs 187763(BRD4), Accession No. Hs 187866(SDFR1), Accession No. Hs 187946(SLC20A1), Accession No. Hs 188501(PAFAH1B2), Accession No. Hs 188614(PLEKHA5), Accession No. Hs 188879(RBM6), Accession No. Hs 188882(NUDT3), Accession No. Hs 189075(PTK9), Accession No. Hs 189119(CXXC5), Accession No. Hs 189329(SMURF1), Accession No. Hs 189716(NDUFAB1), Accession No. Hs 189772(CCT2), Accession No. Hs 190028(GSTO1), Accession No. Hs 190086(MRCL3), Accession No. Hs 190334(RAP1A), Accession No. Hs 190384(COPS4), Accession No. Hs 190722(HSPC142), Accession No. Hs 190904(STRN4), Accession No. Hs 191186(TTC17), Accession No. Hs 191346(SEPT7), Accession No. Hs 191518(DHX9), Accession No. Hs 191987(UBE2J2), Accession No. Hs 192316(CDC2L1), Accession No. Hs 192374(TRA1), Accession No. Hs 192425(EIF3S8), Accession No. Hs 193118(RAI17), Accession No. Hs 193163(BIN1), Accession No. Hs 193491(TUBB6), Accession No. Hs 194329(TCEAL4), Accession No. Hs 194718(ZNF265), Accession No. Hs 195464(FLNA), Accession No. Hs 195642(C17orf27), Accession No. Hs 196983(SSFA2), Accession No. Hs 198281(PKM2), Accession No. Hs 199561(RANBP2), Accession No. Hs 199625(HAX1), Accession No. Hs 200063(HDAC7A), Accession No. Hs 200600(SCAMP3), Accession No. Hs 200804(SDCBP), Accession No. Hs 201253(ch-TOG), Accession No. Hs 201390(WDR45L), Accession No. Hs 201712(GLG1), Accession No. Hs 202011(GK001), Accession No. Hs 202085(VDAC1), Accession No. Hs 202166(HNRPH1), Accession No. Hs 202179(SMN2), Accession No. Hs 203099(KIAA0261), Accession No. Hs 203910(SGTA), Accession No. Hs 204041(AHSA1), Accession No. Hs 204773(MEP50), Accession No. Hs 205163(MRPL3), Accession No. Hs 206500(CTTN), Accession No. Hs 206824(MGC71993), Accession No. Hs 208597(CTBP1), Accession No. Hs 209983(STMN1), Accession No. Hs 210469(ELMO2), Accession No. Hs 210532(KIAA0141), Accession No. Hs 211463(DNM2), Accession No. Hs 211594(PSMC4), Accession No. Hs 211914(NDUFS7), Accession No. Hs 212102(TXNDC7), Accession No. Hs 212395(CIZ1), Accession No. Hs 213061(NUCKS), Accession No. Hs 213470(PSMB7), Accession No. Hs 213541, Accession No. Hs 213666(KIAA0460), Accession No. Hs 213724(SUPT16H), Accession No. Hs 216653(FBXO9), Accession No. Hs 220950(FOXO3A), Accession No. Hs 221847(SLC38A2), Accession No. Hs 222510(DAZAP1), Accession No. Hs 223141(DDX21), Accession No. Hs 224607(SDC1), Accession No. Hs 226007(RDH11), Accession No. Hs 226117(H1F0), Accession No. Hs 226755(YWHAH), Accession No. Hs 227067(ATAD3A), Accession No. Hs 227253(TOMM70A), Accession No. Hs 227777(PTP4A1), Accession No. Hs 229641(PC4), Accession No. Hs 231295(PITPNC1), Accession No. Hs 231616(HSPC023), Accession No. Hs 232194(KIAA0174), Accession No. Hs 232543(PDCD4), Accession No. Hs 233458(NFYC), Accession No. Hs 233552(CDC2L5), Accession No. Hs 233952(PSMA7), Accession No. Hs 234521(MAPKAPK3), Accession No. Hs 236030(SMARCC2), Accession No. Hs 237536(MGC20781), Accession No. Hs 237971(XTP3TPA), Accession No. Hs 238839(SCYL1), Accession No. Hs 240170(MGC2731), Accession No. Hs 241336(ATPIF1), Accession No. Hs 241543(POLDIP2), Accession No. Hs 241558(ARIH2), Accession No. Hs 241575(GNPTG), Accession No. Hs 241576(DERL1), Accession No. Hs 241579(SERPINH1), Accession No. Hs 242458(SPG21), Accession No. Hs 242947(DGKI), Accession No. Hs 246112(ASCC3L1), Accession No. Hs 246310(ATP5J), Accession No. Hs 246413(CPNE1), Accession No. Hs 246781(FBXO11), Accession No. Hs 247077(RHOA), Accession No. Hs 247186(FBS1), Accession No. Hs 247975(HSPD1), Accession No. Hs 248267(MPST), Accession No. Hs 248941(TAF9), Accession No. Hs 49600(DLGAP4), Accession No. Hs 250009(ARL10C), Accession No. Hs 250429(SUPT6H), Accession No. Hs 250758(PSMC3), Accession No. Hs 250899(HSBP1), Accession No. Hs 250905(LOC51234), Accession No. Hs 251531(PSMA4), Accession No. Hs 252457(MVD), Accession No. Hs 252713(TTC15), Accession No. Hs 252967 DKFZp566C0424), Accession No. Hs 253726(PAPOLA), Accession No. Hs 253903(STOM), Accession No. Hs 254042(BAT1), Accession No. Hs 255015(VPS24), Accession No. Hs 255093(PFKL), Accession No. Hs 255932(XRN2), Accession No. Hs 255935(BTG1), Accession No. Hs 255973(CRI1), Accession No. Hs 256301(MGC13170), Accession No. Hs 256549(NUBP2), Accession No. Hs 257008(PLD3), Accession No. Hs 257341(SAV1), Accession No. Hs 57761(SH3BP5), Accession No. Hs 258551(DNPEP), Accession No. Hs 258563(FEZ2), Accession No. Hs 258798 C10orf86), Accession No. Hs 259461(PALM2-AKAP2), Accession No. Hs 260603(PIP5K2B), Accession No. Hs 262823(FLJ10326), Accession No. Hs 265829(ITGA3), Accession No. Hs 268488(KIAA1185), Accession No. Hs 268530(GPS1), Accession No. Hs 268742(C13orf12), Accession No. Hs 268849(GLO1), Accession No. Hs 268939(MATR3), Accession No. Hs 269528(MAK3), Accession No. Hs 269577(PTPRA), Accession No. Hs 269782(GNAQ), Accession No. Hs 269944(MTCH2), Accession No. Hs 270291(ACTN4), Accession No. Hs 270428(SUCLG1), Accession No. Hs 270525(LASS5), Accession No. Hs 270869(ZNF410), Accession No. Hs 271135(ATP5C1), Accession No. Hs 271695(NOB1P), Accession No. Hs 272062(PTPRF), Accession No. Hs 272168(TDE1), Accession No. Hs 272630(ATP6V1D), Accession No. Hs 272927(SEC23A), Accession No. Hs 273077(TMEM14B), Accession No. Hs 274184(TFE3), Accession No. Hs 274772(C15orf15), Accession No. Hs 274873(CARS), Accession No. Hs 275243(S100A6), Accession No. Hs 275775(SEPP1), Accession No. Hs 275865(PCNP), Accession No. Hs 276878(NUP93), Accession No. Hs 277035(MGLL), Accession No. Hs 277517(C11orf2), Accession No. Hs 278186(ARHGEF1), Accession No. Hs 278362(MEA), Accession No. Hs 278426(PDAP1), Accession No. Hs 278429(C9orf78), Accession No. Hs 278500(GNPDA1), Accession No. Hs 278569(SNX17), Accession No. Hs 278573(CD59), Accession No. Hs 278721(SLC39A7), Accession No. Hs 279061(C17orf25), Accession No. Hs 279245(TACC1), Accession No. Hs 279257(PCMT1), Accession No. Hs 279413(POLD1), Accession No. Hs 279529(PX19), Accession No. Hs 279583(DREV1), Accession No. Hs 279623(SEPX1), Accession No. Hs 279640(TPR), Accession No. Hs 279652(MRPL4), Accession No. Hs 279669(TUBG1), Accession No. Hs 279696(SUMF2), Accession No. Hs 279806(DDX5), Accession No. Hs 79836(COMMD9), Accession No. Hs 279920(YWHAB), Accession No. Hs 279929(TMED9), Accession No. Hs 80202(SBF1), Accession No. Hs 280342(PRKAR1A), Accession No. Hs 280378(SNRPB2), Accession No. Hs 282410(CALM1), Accession No. Hs 282700(SPCS2), Accession No. Hs 282901(RNPC2), Accession No. Hs 282998(RBM9), Accession No. Hs 283111(C14orf124), Accession No. Hs 283454(BNIP2), Accession No. Hs 283521(RHEB), Accession No. Hs 283610(APG4B), Accession No. Hs 283652(IDI1), Accession No. Hs 283739(UBQLN4), Accession No. Hs 284208(ANKRD25), Accession No. Hs 284279(HMOX2), Accession No. Hs 284286(MRPS24), Accession No. Hs 284491(PDXK), Accession No. Hs 285354(MAX), Accession No. Hs 285976(LASS2), Accession No. Hs 286221(ARF1), Accession No. Hs 286226(MYO1C), Accession No. Hs 288193(KPNA4), Accession No. Hs 288856(PFDN5), Accession No. Hs 288969(HSCARG), Accession No. Hs 289008(C6orf68), Accession No. Hs 289092(COTL1), Accession No. Hs 289123(DCTN2), Accession No. Hs 289271(CYC1), Accession No. Hs 290243(GBF1), Accession No. Hs 290404(SLC25A3), Accession No. Hs 290758(DDB1), Accession No. Hs 291587(ARID1B), Accession No. Hs 292026(EIF4E2), Accession No. Hs 292063(EIF4B), Accession No. Hs 292078(LARP), Accession No. His 292265(ZMYND11), Accession No. Hs 292457, Accession No. Hs 292493(G22P1), Accession No. Hs 292524 (CCNH), Accession No. Hs 292579(PTDSS1), Accession No. Hs 293563(FLJ12666), Accession No. Hs 295917(ATP6V1B2), Accession No. Hs 297324(TIMP3), Accession No. Hs 298198(CKLFSF3), Accession No. Hs 298280(ATP5A1), Accession No. Hs 298654(DUSP6), Accession No. Hs 299002(FBL), Accession No. Hs 299055(GDI2), Accession No. Hs 300141(RPL39), Accession No. Hs 300684(RCP9), Accession No. Hs 300772(TPM2), Accession No. Hs 300816 RABIB), Accession No. Hs 300834(GALNT2), Accession No. Hs 301404(RB3M3), Accession No. Hs 301412(Ufc1), Accession No. Hs 302742(MRPS6), Accession No. Hs 302903(UBE21), Accession No. Hs 303676(G3BP2), Accession No. Hs 304192(DSTN), Accession No. Hs 304682(CST3), Accession No. Hs 306123(MAGEF1), Accession No. Hs 306242(RANBP9), Accession No. Hs 306329(ZA20D3), Accession No. Hs 306425(IBTK), Accession No. Hs 308122(ITPK1), Accession No. Hs 308340(NUP188), Accession No. Hs 308709(GRP58), Accession No. Hs 309090(SFRS7), Accession No. Hs 309231(C6orf153), Accession No. Hs 309641(RNF11), Accession No. Hs 309753(STARD3NL), Accession No. Hs 309849(C14orf159), Accession No. Hs 310542(TOMM40), Accession No. Hs 310645(RAB1A), Accession No. Hs 311072(MRPS35), Accession No. Hs 311346(CMAS), Accession No. Hs 311609(DDX39), Accession No. Hs 311640(RPS27A), Accession No. Hs 312098(ADAM15), Accession No. Hs 313847(TXNDC11), Accession No. Hs 314263(BAZ2A), Accession No. Hs 314359(EIF3S12), Accession No. Hs 315177(IFRD2), Accession No. Hs 315230(GC20), Accession No. Hs 319334(NASP), Accession No. Hs 321391(MGC4549), Accession No. Hs 321541(RAB11A), Accession No. Hs 323363(APG9L1), Accession No. Hs 323489(F1J20758), Accession No. Hs 324250(NDUFB2), Accession No. Hs 324844(VKORC1), Accession No. Hs 325650(EHD2), Accession No. Hs 326387(MORF4L2), Accession No. Hs 330384(CORO1C), Accession No. Hs 331431(SCC-112), Accession No. Hs 333388(EEF1D), Accession No. Hs 333579(HSPC152), Accession No. Hs 333786(PSMA2), Accession No. Hs 333823(MRPL13), Accession No. Hs 334017(K-ALPHA-1), Accession No. Hs 334479(TRAF7), Accession No. Hs 334534(GNS), Accession No. Hs 334587(RBPMS), Accession No. Hs 334713(BMSC-UbP), Accession No. Hs 334851(LASP1), Accession No. Hs 334868(PPP2R5E), Accession No. Hs 335003(ANKRD11), Accession No. Hs 335057(SEPT2), Accession No. Hs 335163(KIAA1102), Accession No. Hs 335918(FDPS), Accession No. Hs 337295(STIP1), Accession No. Hs 337766(TXNRD1), Accession No. Hs 339278(COPB), Accession No. Hs 339639(COX7A2L), Accession No. Hs 339697(GRINA), Accession No. Hs 343911(E124), Accession No. Hs 345694(KCMF1), Accession No. Hs 346868(EBNA1BP2), Accession No. Hs 348418(DR1), Accession No. Hs 349656(SCARB2), Accession No. Hs 350194(ZMAT2), Accession No. Hs 350229(CASC3), Accession No. Hs 350268(IRF2BP2), Accession No. Hs 350364(C9orf100S), Accession No. Hs 350927(SLC25A6), Accession No. Hs 351099(FLJ10241), Accession No. Hs 351296(LOC51035), Accession No. Hs 351316(TM4SF1), Accession No. Hs 351474(PAQR4), Accession No. Hs 351680, Accession No. Hs 351875(COX6C), Accession No. Hs 352341(STCH), Accession No. Hs 352656(GHITM), Accession No. Hs 352768(PSMB1), Accession No. Hs 354056(POR), Accession No. Hs 355141(TNIP1), Accession No. Hs 355606(MGC23909), Accession No. Hs 355643(RNPS1), Accession No. Hs 355708(FLJ20507), Accession No. Hs 355750(MGC5306), Accession No. Hs 355753(DKFZp586M1819), Accession No. Hs 355867(MARS), Accession No. Hs 355927(VDAC2), Accession No. Hs 355934(SFPQ), Accession No. Hs 355983(BZW1), Accession No. Hs 356061(MAP1LC3B), Accession No. Hs 356096(FLJ10350), Accession No. Hs 356190(UBB), Accession No. Hs 356270(SDHD), Accession No. Hs 356285(HMGN1), Accession No. Hs 356331(PPIA), Accession No. Hs 356366(RPS2), Accession No. Hs 356371(RPL28), Accession No. Hs 356377(LOC149603), Accession No. Hs 356467(MGC2747), Accession No. Hs 356501(PHF6), Accession No. Hs 356502(RPLP1), Accession No. Hs 356549(SNRPD3), Accession No. Hs 356630(NUTF2), Accession No. Hs 356647(SNX6), Accession No. Hs 356654(PSMC1), Accession No. Hs 56766( ), Accession No. Hs 356769(MAN2B1), Accession No. Hs 356799, Accession No. Hs 357901(SOX4), Accession No. Hs 362728(SEP15), Accession No. Hs 365116(U2AF1), Accession No. Hs 368084(LRPPRC), Accession No. Hs 368149(CCT7), Accession No. Hs 368157(PYGB), Accession No. Hs 368240(DYRKIA), Accession No. Hs 68264(PPP2R5C), Accession No. Hs 368376(SRPR), Accession No. Hs 368402(LOC51337), Accession No. Hs 368404(EXT2), Accession No. Hs 368525(PDLIM1), Accession No. Hs 368598(LEREPO4), Accession No. Hs 368934(MGC40157), Accession No. Hs 368985(TRIP12), Accession No. Hs 369017(RAB2), Accession No. Hs 369052(SELT), Accession No. Hs 369068(DNCL12), Accession No. Hs 369125(PSMD14), Accession No. Hs 369285(DKFZP434B168), Accession No. Hs 369356(MLL5), Accession No. Hs 369606(CPSF6), Accession No. Hs 369607(GAK), Accession No. Hs 369614(COPS2), Accession No. Hs 369615(FLJ20551), Accession No. Hs 369761(DAZAP2), Accession No. Hs 369785(MGC2749), Accession No. Hs 369920(RAP1B), Accession No. Hs 370024(SEC31L1), Accession No. Hs 370247(APLP2), Accession No. Hs 370292(BCCIP), Accession No. Hs 370312(FNTA), Accession No. Hs 370408(COMT), Accession No. Hs 370581(CAP1), Accession No. Hs 370770(XPO1), Accession No. Hs 370771(CDKN1A), Accession No. Hs 370895(RPN2), Accession No. Hs 370927(PRO1855), Accession No. Hs 370937(TAPBP), Accession No. Hs 371001(EIF3S9), Accession No. Hs 371416(CARM1), Accession No. Hs 371563(RAB14), Accession No. Hs 371788(DKFZP547E1010), Accession No. Hs 371889(ATP1A1), Accession No. Hs 372003(C9orf10), Accession No. Hs 372050(SMAP-5), Accession No. Hs 372286(CUL3), Accession No. Hs 372331(SPTAN1), Accession No. Hs 372541(KBTBD2), Accession No. Hs 372616(ARL1), Accession No. Hs 372914(NDRG1), Accession No. Hs 373550(TGIF), Accession No. Hs 373741(HM13), Accession No. Hs 373763(HNRPR), Accession No. Hs 373952(CAMTA2), Accession No. Hs 373959(VGLL4), Accession No. Hs 374043(ASXL1), Accession No. Hs 374257(SIAT4A), Accession No. Hs 374378(CKS1B), Accession No. Hs 374477(EWSR1), Accession No. Hs 374503(MORF4L1), Accession No. Hs 374588(RPL17). Accession No. Hs 374596(TPT1), Accession No. Hs 374650(IFITM3), Accession No. Hs 374973(PRPF4), Accession No. Hs 375001(TLN1), Accession No. Hs 375108(CD24), Accession No. Hs 375217(RNF31), Accession No. Hs 376046(BTN3A2), Accession No. Hs 376933(GUK1), Accession No. Hs 377155(LYRIC), Accession No. Hs 378103(RPS5), Accession No. Hs 378532(HBS1L), Accession No. Hs 378808(eIF2A), Accession No. Hs 380403(PCGF4), Accession No. Hs 380774(DDX3X), Accession No. Hs 380953(RPL38), Accession No. Hs 380973(SUMO2), Accession No. Hs 381008(HLA-E), Accession No. Hs 381058(KIAA0146). Accession No. Hs 381072(PPIF), Accession No. Hs 381123(RPL21), Accession No. Hs 381126(RPS14), Accession No. Hs 381189(CBX3), Accession No. Hs 381219, Accession No. Hs 381256(GLTP), Accession No. Hs 382044(MRPS2), Accession No. Hs 382168(NCOA3), Accession No. Hs 385913(ANP32E), Accession No. Hs 385986(UBE2B), Accession No. Hs 386434(ANXA7), Accession No. Hs 386465(CHERP), Accession No. Hs 386939(USP7), Accession No. Hs 387208(FAU), Accession No. Hs 387804(PABPC1), Accession No. Hs 388034(RXRB), Accession No. Hs 388654(ATP6V1G1), Accession No. Hs 388664(RPL11), Accession No. Hs 388739(XRCC5), Accession No. Hs 388927(YY1), Accession No. Hs 388956(C19orf22), Accession No. Hs 389037(MCM3APAS), Accession No. Hs 389107(ATP6V0C), Accession No. Hs 389171(PINK1). Accession No. Hs 389649(DDX48), Accession No. Hs 389734(TCEAL8), Accession No. Hs 389996(CHCHD2), Accession No. Hs 390667(GSTK1), Accession No. Hs 393201(ACTR2), Accession No. Hs 395482(PTK2), Accession No. Hs 396644(PAIP2), Accession No. Hs 396740(NIP30), Accession No. Hs 396783(SLC9A3R1), Accession No. Hs 397609(RPS16), Accession No. Hs 399800(AKAP8L), Accession No. Hs 400295(RPL30), Accession No. Hs 401509(RBM10), Accession No. Hs 401903(COX5A), Accession No. Hs 401929(RPL10), Accession No. Hs 403917(STK24), Accession No. Hs 404056(EIF3S1), Accession No. Hs 404321(GARS), Accession No. Hs 405144(SFRS3), Accession No. Hs 405410(OGT), Accession No. Hs 405514(LOC284058), Accession No. Hs 405590(EIF3S6), Accession No. Hs 405880(MRPS21), Accession No. Hs 405942(LOC339229), Accession No. Hs 406062(NDUFA11), Accession No. Hs 406068(UBE2M), Accession No. Hs 406096(ZA20D2), Accession No. Hs 406277(SF3A1), Accession No. Hs 406300(RPL23), Accession No. Hs 406423(SF3B2), Accession No. Hs 406510(ATP5B), Accession No. Hs 406520(LOC389541), Accession No. Hs 406534(HMG20B), Accession No. Hs 406590(PGR1), Accession No. Hs 406620(RPS10), Accession No. Hs 406683(RPS15), Accession No. Hs 406799(RAB18), Accession No. Hs 406840(SLC35A4), Accession No. Hs 407368(C19orf13), Accession No. Hs 407580(PKP4), Accession No. Hs 407995(MIF), Accession No. Hs 408018(RPL36), Accession No. Hs 408073(RPS6), Accession No. Hs 408236(TXNL5), Accession No. Hs 408257(NDUFS6), Accession No. Hs 408293(KAB), Accession No. Hs 408324(FLJ10769), Accession No. Hs 408428(CHES1), Accession No. Hs 408581(SVIL), Accession No. Hs 408909(GOLPH3), Accession No. Hs 409140(ATP50), Accession No. Hs 409223(SSR4), Accession No. Hs 409230(AGPAT1), Accession No. Hs 409834(PHPT1), Accession No. Hs 410197(IDH3G), Accession No. Hs 410596(HAN11), Accession No. Hs 410817(RPL13), Accession No. Hs 411480(AUP1), Accession No. Hs 411641(ElF4EBP1), Accession No. Hs 411847(MAPK6), Accession No. Hs 412103(EFHA1), Accession No. Hs 412117(ANXA6), Accession No. Hs 412196(ESRRBL1), Accession No. Hs 412433(AIP), Accession No. Hs 412468(KLHDC3), Accession No. Hs 412842(C10orf7), Accession No. Hs 413036(WBSCR22), Accession No. Hs 413482(C21orf33), Accession No. Hs 414579(SCOTIN), Accession No. Hs 415342(KIAA1049), Accession No. Hs 416049(TNPO2), Accession No. Hs 416436(TRIM50A), Accession No. Hs 417004(S100A11), Accession No. Hs 417029(DERP6), Accession No. Hs 418123(CTSL), Accession No. Hs 418175(VPS28), Accession No. Hs 418233(MRPL24), Accession No. Hs 418450(MRPL11), Accession No. Hs 418533(BUB3), Accession No. Hs 418668(ATP5D), Accession No. Hs 419640(PARK7), Accession No. Hs 420269(COL6A2), Accession No. Hs 420272(H2AFY), Accession No. Hs 421257(RPL7), Accession No. Hs 421509(CCT4). Accession No. Hs 422113(ZNF511), Accession No. Hs 423935(RDBP), Accession No. Hs 423968(TTC11), Accession No. Hs 424126(SERF2), Accession No. Hs 424908(LSM5), Accession No. Hs 425777(UBE2L6), Accession No. Hs 426296(C10orf104), Accession No. Hs 426359(DKFZp564J157), Accession No. Hs 429052(ITGB1), Accession No. Hs 429353(SEPN1), Accession No. Hs 429581(RTN4), Accession No. Hs 429819(PITPNA), Accession No. Hs 429839(MGC23908), Accession No. Hs 430425(GNB1), Accession No. Hs 430551(IQGAP1), Accession No. Hs 430606(CS), Accession No. Hs 430657(ARF5), Accession No. Hs 430733(CLNS1A), Accession No. Hs 431101(GNG12), Accession No. Hs 431367(C6orf55), Accession No. Hs 431498(FOXP1), Accession No. Hs 431550(MAP4K4), Accession No. Hs 431668(COX6B1), Accession No. Hs 431850(MAPK1), Accession No. Hs 431861(PPP5C), Accession No. Hs 431926 NFKB1), Accession No. Hs 432121(PRDX2), Accession No. Hs 432438(EML4), Accession No. Hs 432491(ESD), Accession No. Hs 432690(SLC39A9), Accession No. Hs 432760(CAPZB), Accession No. Hs 432898(RPL4), Accession No. Hs 432976(NR1H2), Accession No. Hs 433154(PLSCR3), Accession No. Hs 433201(CDK2AP1), Accession No. Hs 433222(NPC2), Accession No. Hs 433291(ARD1), Accession No. Hs 433307(BCKDHA), Accession No. Hs 433343(SRRM2), Accession No. Hs 433345, Accession No. Hs 433419(COX4I1), Accession No. Hs 433512(ACTR3), Accession No. Hs 433529(RPS11), Accession No. Hs 433540(DNAJC8), Accession No. Hs 433573(Bles03), Accession No. Hs 433615(TUBB2), Accession No. Hs 433701(RPL37A), Accession No. Hs 433722(KIAA1967), Accession No. Hs 33732(CLK1), Accession No. Hs 433750(EIF4G1), Accession No. Hs 433759(BANF1), Accession No. Hs 433795(SHC1), Accession No. Hs 433863(PBP), Accession No. Hs 433901(COX8A), Accession No. Hs 433951(GPX4), Accession No. Hs 434102(HMGB1), Accession No. Hs 434207(HARS2), Accession No. His 434219(ANKHD1), Accession No. Hs 434401(ZNF638), Accession No. Hs 434937(PPIB), Accession No. Hs 434953(HMGB2), Accession No. Hs 434980(APP), Accession No. Hs 435044(TBC1D22A), Accession No. Hs 435064(KIAA1608), Accession No. Hs 435120(KIF1C), Accession No. Hs 435136(TXN), Accession No. Hs 435166(LBR), Accession No. Hs 435231(ZFR), Accession No. Hs 435255(UBXD1), Accession No. Hs 435326(ACTL6A), Accession No. Hs 435512(PPP3CA), Accession No. Hs 435535(ZNF395), Accession No. Hs 435610(WAC), Accession No. Hs 435741(GCSH), Accession No. Hs 435759(THAP4), Accession No. Hs 435771(API5), Accession No. Hs 435841(TNRC15), Accession No. Hs 435850(LYPLA1), Accession No. Hs 435933(PHF10), Accession No. Hs 435948(ATAD1), Accession No. Hs 435952(CDK5RAP1), Accession No. Hs 435974(MTHFD1), Accession No. Hs 436035(TUBA6), Accession No. Hs 436093(BAT2), Accession No. Hs 436204(ZNF289), Accession No. Hs 436298(EMP1), Accession No. Hs 436405(IDH3B), Accession No. Hs 436437(ALDH2), Accession No. Hs 436446(ARMET), Accession No. Hs 436500(DBNL), Accession No. Hs 436568(CD74), Accession No. Hs 436578(POLR2F), Accession No. Hs 436657(CLU), Accession No. Hs 436687(SET), Accession No. Hs 436803(VBP1), Accession No. Hs 437056(SUPT5H), Accession No. Hs 437060(CYCS), Accession No. Hs 437110(ANXA2), Accession No. Hs 437178(ACADVL), Accession No. Hs 437256(GRINL1A), Accession No. Hs 437277(MGAT4B), Accession No. Hs 437367(GBAS), Accession No. Hs 437388(PIGT), Accession No. Hs 437403(PP), Accession No. Hs 437594(RPLP2), Accession No. Hs 437638(XBP1), Accession No. Hs 437779(C11orf10), Accession No. Hs 437831(C14orf32), Accession No. Hs 438072(UNC84A), Accession No. Hs 438219(GPS2), Accession No. Hs 438429(RPS19), Accession No. Hs 438678(TALDO1), Accession No. Hs 438720(MCM7), Accession No. Hs 438970(TBL1XR1), Accession No. Hs 438974(CUTL1), Accession No. Hs 439480(RBM5), Accession No. Hs 439481(SUPT4H1), Accession No. Hs 439548(FLJ22875), Accession No. Hs 439552, Accession No. Hs 439815(HBXIP), Accession No. Hs 440382(RFP), Accession No. Hs 440544(CLIC4), Accession No. Hs 440599(DDX1). Accession No. Hs 440604(PSMD7), Accession No. Hs 440899(TTYH3), Accession No. Hs 440932(SEPT9), Accession No. Hs 440960(RAD23A), Accession No. Hs 440961(CAST), Accession No. Hs 441072(POLR2L), Accession No. Hs 441550(C20orf22), Accession No. Hs 442344(IRS2), Accession No. Hs 442798(RNF10), Accession No. Hs 443134(GBA2), Accession No. Hs 443379(PSMD11), Accession No. Hs 443837(NPEPPS), Accession No. Hs 443914(SOD1), Accession No. Hs 444279(DKFZp761C169), Accession No. Hs 444356(GRB2), Accession No. Hs 444468(CTDSP1). Accession No. Hs 444472(SDHC), Accession No. Hs 444569(VMP1), Accession No. Hs 444673(CRR9), Accession No. Hs 444724(AZI2), Accession No. Hs 444818(CGGBP1), Accession No. Hs 444931(CRSP6), Accession No. Hs 444969(C2orf4), Accession No. Hs 444986(METAP2), Accession No. Hs 445081(NS5ATP13TP2), Accession No. Hs 445351(LGALS1), Accession No. Hs 445394(VPS29), Accession No. Hs 445498(SKIIP), Accession No. Hs 445511(RIOK3), Accession No. Hs 445570(CD63), Accession No. Hs 445803(DC2), Accession No. Hs 445893(KHDRBS1), Accession No. Hs 445977(GTF3A), Accession No. Hs 446017(WSB1), Accession No. Hs 446091(WTAP), Accession No. Hs 446123(CAPZA2), Accession No. Hs 446149(LDHB), Accession No. Hs 446260(PSMA6), Accession No. Hs 446336(PXN), Accession No. Hs 446345(FTH1), Accession No. Hs 446414(CD47), Accession No. Hs 446427(OAZ1), Accession No. Hs 446445(YIF1), Accession No. Hs 446450(ITM2B), Accession No. Hs 446574(TMSB10), Accession No. Hs 446588(RPS13), Accession No. Hs 446623(HNRPL), Accession No. Hs 446628(RPS4X), Accession No. Hs 446641(ARAF), Accession No. Hs 446852(EIF3S6IP), Accession No. Hs 447477(ST13), Accession No. Hs 447492(PGAM1), Accession No. Hs 447547(VPS35), Accession No. Hs 48226(RPLP0), Accession No. Hs 448588(NGFRAP1), Accession No. Hs 448646(RPL27A), Accession No. Hs 448879, Accession No. Hs 449114(HNRPC), Accession No. Hs 449171(HNRPK), Accession No. Hs 454534(USF2), Accession No. Hs 454699(IL6ST), Accession No. Hs 456507(PKD1-like), Accession No. Hs 456557(FLJ10597), Accession No. Hs 458320(DC12), Accession No. Hs 458358(TSPYL1), Accession No. Hs 458414(IFITM1), Accession No. Hs 458458(C19orf27), Accession No. Hs 458747(ANP32A), Accession No. Hs 459106(OAZIN), Accession No. Hs 459149(BTBD1), Accession No. Hs 459174(FLJ23790), Accession No. Hs 459211 (AKAP13), Accession No. Hs 459596(MPG). Accession No. Hs 459649(CLCN7), Accession No. Hs 459927(PTMA), Accession No. Hs 459940(LITAF), Accession No. Hs 460238(SH3GLB2), Accession No. Hs 460317(ALS4), Accession No. Hs 460336(GGA2), Accession No. Hs 460468(XPO6), Accession No. Hs 460499(ATXN2L), Accession No. Hs 460574(LOC124446), Accession No. Hs 460923(CNOT1), Accession No. Hs 460929(GOT2), Accession No. Hs 460978(APPBP1), Accession No. Hs 461047(G6PD), Accession No. Hs 461131(CYB5-M), Accession No. Hs 461361(CFDP1), Accession No. Hs 461379(GABARAPL2), Accession No. Hs 461722(HSPC176), Accession No. Hs 461777(PCOLN3), Accession No. Hs 461896(CRK), Accession No. Hs 461925(RPA1), Accession No. Hs 462035(UBE2G1), Accession No. Hs 462086(RIP), Accession No. Hs 462306(UBE2S), Accession No. Hs 462316(TTC19), Accession No. Hs 462492(USP22), Accession No. Hs 462550(PIGS), Accession No. Hs 462956(PPARBP), Accession No. Hs 462998(IGFBP4), Accession No. Hs 463010(SMARCE1), Accession No. Hs 463035(FKBP10), Accession No. Hs 463041(RERE), Accession No. Hs 463059(STAT3), Accession No. Hs 463295(CDC27), Accession No. Hs 463506(AKAP1), Accession No. Hs 463702(BCAS3), Accession No. Hs 463797(C1orf33), Accession No. Hs 464071(PGD), Accession No. Hs 464137(ACOX1), Accession No. Hs 464210(SYNGR2), Accession No. Hs 464336(P4HB), Accession No. Hs 464438(AGTRAP), Accession No. Hs 464472(MRLC2), Accession No. Hs 464595(PPP4R1), Accession No. Hs 464652(TNFSF5IP1), Accession No. Hs 464912(P15RS), Accession No. Hs 465224(NARS), Accession No. Hs 465374(EFHD2), Accession No. Hs 465498(TXNL4A), Accession No. Hs 465529(MIDN), Accession No. Hs 465543(BTBD2), Accession No. Hs 465627(MAP2K2), Accession No. Hs 465645(C19orf10), Accession No. Hs 465808(HNRPM), Accession No. Hs 465849(PIN1), Accession No. Hs 465924(SDHB), Accession No. Hs 466044(PKN1), Accession No. Hs 466088(TPM4), Accession No. Hs 466148(NR2F6), Accession No. Hs 466471(GPI), Accession No. Hs 466693(SIRT2), Accession No. Hs 466766(LTBP4), Accession No. Hs 466775(SNRPA), Accession No. Hs 467084(EIF4G3), Accession No. Hs 467097(SNRP70), Accession No. Hs 467192(PPP2R1A), Accession No. Hs 467279(LENG4), Accession No. Hs 467284(RPS9), Accession No. Hs 467408(TRIM28), Accession No. Hs 467637(CDC42), Accession No. Hs 467696(HPCAL1), Accession No. Hs 467701(ODC1), Accession No. Hs 467807(LAPTM4A), Accession No. Hs 467824(PUM2), Accession No. Hs 467960(RAB10), Accession No. Hs 468018(PPP1CB), Accession No. Hs 468415(PIGF), Accession No. Hs 468442(CALM2), Accession No. Hs 468760(AFTIPHILIN), Accession No. Hs 469022(DGUOK), Accession No. Hs 469171(DKFZP564D0478), Accession No. Hs 469331(STARD7), Accession No. Hs 469820(RALB), Accession No. Hs 469863(YWHAZ), Accession No. Hs 469925(FLJ14346), Accession No. Hs 469970(SFRS4), Accession No. Hs 470091(YWHAE), Accession No. Hs 470233(ARL5), Accession No. Hs 470417, Accession No. Hs 470477(PTP4A2), Accession No. Hs 470577(EIF2S2), Accession No. Hs 470588(KPNA6), Accession No. Hs 470943(STAT1), Accession No. Hs 471011(SF3B1), Accession No. Hs 471104(NOP5/NOP58), Accession No. Hs 471207(NDUFS1), Accession No. Hs 471441(PSMB2), Accession No. Hs 471461(ACSL3), Accession No. Hs 471593(CAB39), Accession No. Hs 471768(MGC4796), Accession No. Hs 471818(M11S1), Accession No. Hs 471851(HDLBP), Accession No. Hs 471873(DTYMK), Accession No. Hs 471933(FKBPIA), Accession No. Hs 471975(C20orf116), Accession No. Hs 472010(PRNP). Accession No. Hs 472024(C20orf30), Accession No. Hs 472031(UBE2D3), Accession No. Hs 472038(CGI-94), Accession No. Hs 472056(SYNCRIP), Accession No. Hs 472119(MKKS), Accession No. Hs 472185(NDUFS5), Accession No. Hs 472213(RRBP1), Accession No. Hs 472330(C20orf3), Accession No. Hs 472475(MACF1), Accession No. Hs 472535(AKIP), Accession No. Hs 472558(SDBCAG84), Accession No. Hs 472651(BLCAP), Accession No. Hs 472737(TOP1), Accession No. Hs 473296(TPD52L2), Accession No. Hs 473583(NSEP1), Accession No. Hs 473648(GART), Accession No. Hs 473721(SLC2A1), Accession No. Hs 473761(RTN3), Accession No. Hs 473788(OTUB1), Accession No. Hs 474005(SUMO3), Accession No. Hs 474010(PTTG1IP), Accession No. Hs 474053(COL6A1), Accession No. Hs 474083(B4GALT2), Accession No. Hs 474213(UFD1L), Accession No. Hs 474584(AKR1A1), Accession No. Hs 474643(HSPC117), Accession No. Hs 474751(MYH9), Accession No. Hs 474833(CSNK1E), Accession No. Hs 474914(RUTBC3), Accession No. Hs 474938(SLC25A17), Accession No. Hs 474949(RBX1), Accession No. Hs 474982(ACO2), Accession No. Hs 475125(ATXN10), Accession No. Hs 475319(LRRFIP2), Accession No. Hs 475382(FLJ22405), Accession No. Hs 475392(LOC55831), Accession No. Hs 475663(RAB5A), Accession No. Hs 475733(TOP2B), Accession No. Hs 475812(SIMP), Accession No. Hs 476018(CTNNB1), Accession No. Hs 476033(TLP19), Accession No. Hs 476179(SMARCC1), Accession No. Hs 476221(IHPK2), Accession No. Hs 76231(IMPDH2), Accession No. Hs 476308(ALAS1), Accession No. Hs 476365(SCP2), Accession No. Hs 476448(FLNB), Accession No. Hs 476706(MRPL37), Accession No. Hs 476930(DKFZP5640123), Accession No. Hs 477157(DULLARD), Accession No. Hs 477789(ATP1B3), Accession No. Hs 477892(GYG), Accession No. Hs 478000(MBNL1), Accession No. Hs 478044(PA2G4), Accession No. Hs 478553(EIF4A2), Accession No. Hs 479208(FBXL5), Accession No. Hs 479264(LAP3), Accession No. Hs 479634(SLC30A9), Accession No. Hs 479693(SFRS11), Accession No. Hs 479728(GAPD), Accession No. Hs 479747(BCAR1), Accession No. Hs 479814(POLR2B), Accession No. Hs 480073(HNRPD), Accession No. Hs 480311(PDLIM5), Accession No. Hs 480465(SCYE1), Accession No. Hs 80653(ANXA5), Accession No. Hs 481571(UQCRH), Accession No. Hs 481720(MYO10), Accession No. Hs 481898(KAT3), Accession No. Hs 482144(RPL26), Accession No. Hs 482363(SLC30A5), Accession No. Hs 482526(TINP1), Accession No. Hs 482868(KIAA0372), Accession No. Hs 483036(PJA2), Accession No. Hs 483067(C5orf13), Accession No. Hs 483305(HINT1), Accession No. Hs 483408(PPP2CA), Accession No. Hs 483454(CNN3), Accession No. Hs 483486(JMJD1B), Accession No. Hs 484138(FBXW11), Accession No. Hs 484188(ATP6V0E), Accession No. 484242(ETEA), Accession No. Hs 484288(DDX41), Accession No. Hs 484363(RNF130), Accession No. Hs 484551(CPM), Accession No. Hs 484813(DEK), Accession No. Hs 485155(RPL35), Accession No. Hs 485195(SORT1), Accession No. Hs 485246(PSMA5), Accession No. Hs 485262(MTCH1), Accession No. Hs 485365(AHCYL1), Accession No. Hs 485616(DST), Accession No. Hs 486542(BCLAF1), Accession No. Hs 487027(VIL2), Accession No. Hs 487054(TCP1), Accession No. Hs 487635(BZW2), Accession No. Hs 487774(HNRPA2B1), Accession No. Hs 488171(KIAA1068), Accession No. Hs 488181(OGDH), Accession No. Hs 488307(DKFZP564K0822), Accession No. Hs 488478(FLJ10099), Accession No. Hs 488671(BAZ1B), Accession No. Hs 489207(ASNS), Accession No. Hs 489284(ARPC1B), Accession No. Hs 489287(CPSF4), Accession No. Hs 489336(SYAP1), Accession No. Hs 489615(PBEF1), Accession No. Hs 490203(CALD1), Accession No. Hs 490394(SSBP1), Accession No. Hs 490415(ZYX), Accession No. Hs 490745(DNAJB6), Accession No. Hs 490795(CHR2SYT), Accession No. Hs 490874(MTX1), Accession No. Hs 491336(ELP3), Accession No. Hs 491359(LMNA), Accession No. Hs 491440(PPP2CB), Accession No. Hs 491494(CCT3), Accession No. Hs 491597(VDAC3), Accession No. Hs 491695(UBE2V2), Accession No. Hs 491745(TCEA1), Accession No. Hs 491988(TRAM1), Accession No. Hs 492236(WDR42A), Accession No. Hs 492314(LAPTM4B), Accession No. Hs 492445(EDD), Accession No. Hs 492599(EIF3S3), Accession No. Hs 492805(CGI-07), Accession No. Hs 493362(AK3L1), Accession No. Hs 493750(WDR40A), Accession No. Hs 494173(ANXA1), Accession No. Hs 494419(LAMP1), Accession No. Hs 494457(NINJ1), Accession No. Hs 494604(ANP32B), Accession No. Hs 494614(XTP2), Accession No. Hs 494691(PFN1), Accession No. Hs 494700(CDW92), Accession No. Hs 494985(FBXW2), Accession No. Hs 495039(NDUFA8), Accession No. Hs 495349(KIAA0515), Accession No. Hs 495471(PMPCA), Accession No. Hs 495605(CD99), Accession No. Hs 495851(MGC4825), Accession No. Hs 495960(ATP6AP2), Accession No. Hs 496068(PCTK1), Accession No. Hs 496098(DKFZp761A052), Accession No. Hs 496271, Accession No. Hs 496487(ATF4), Accession No. Hs 496646(IL13RA1), Accession No. Hs 496684(LAMP2), Accession No. Hs 497183(IVNSIABP), Accession No. Hs 497599(WARS), Accession No. Hs 497692(C1orf48), Accession No. Hs 497893(ENAH), Accession No. Hs 498239(FH), Accession No. Hs 498313(ADSS), Accession No. Hs 498317(PNAS-4), Accession No. Hs 498455(KIAA0217), Accession No. Hs 498548(RBM17), Accession No. Hs 498727(DHCR24), Accession No. Hs 499145(YME1L1), Accession No. Hs 499158(GGA1), Accession No. Hs 499594(TIMM23), Accession No. Hs 499833(C10orf74), Accession No. Hs 499891(HNRPH3), Accession No. Hs 499925(VPS26), Accession No. Hs 499960(SARA1), Accession No. Hs 500067(PPP3CB), Accession No. Hs 500101(VCL), Accession No. Hs 500375(ENTPD6), Accession No. Hs 500409(GLUD1), Accession No. Hs 500546(IDE), Accession No. Hs 500674(SMBP), Accession No. Hs 500775(ZNF207), Accession No. Hs 500842(MGEA5), Accession No. Hs 500874(CUEDC2), Accession No. Hs 501012(ADD3), Accession No. Hs 501023(MXI1), Accession No. Hs 501203(TIAL1), Accession No. Hs 501293(BSG), Accession No. Hs 501309(CIRBP), Accession No. Hs 501353(PLEKHJ1), Accession No. Hs 501376(UROS), Accession No. Hs 501420(NCLN), Accession No. Hs 501629(IER2), Accession No. Hs 501684(NAP1L4), Accession No. Hs 501735(STIM1). Accession No. Hs 501853(C11orf15), Accession No. Hs 501924(USP47), Accession No. Hs 501991(MLSTD2), Accession No. Hs 502302(CAT), Accession No. Hs 502328(CD44), Accession No. Hs 502461(DGKZ), Accession No. Hs 502528(NDUFS3), Accession No. Hs 502630(C11orf31), Accession No. Hs 502659(RHOC), Accession No. Hs 502705(PRP19), Accession No. Hs 502745(FADS2), Accession No. Hs 502769(SLC3A2), Accession No. Hs 502773(MTCBP-1), Accession No. Hs 502823(PRDX5), Accession No. Hs 502829(SF1), Accession No. Hs 502836(ARL2), Accession No. Hs 502842(CAPN1), Accession No. Hs 502872(MAP3K11), Accession No. Hs 502876(RHOB), Accession No. Hs 503093(ZFP36L2), Accession No. Hs 503222(RAB6A), Accession No. Hs 503251(PME-1), Accession No. Hs 503597(HSPC148), Accession No. Hs 503709(PORIMIN), Accession No. Hs 503716(MGC2714), Accession No. Hs 503787(DARS), Accession No. Hs 504237 (ITM1), Accession No. Hs 504517(RPS27), Accession No. Hs 504613(PTMS), Accession No. Hs 504620(REA), Accession No. Hs 504687(MYL9), Accession No. Hs 504828(DDX47), Accession No. Hs 504895(STRAP), Accession No. Hs 505033(KRAS2), Accession No. Hs 505059(PSMD4), Accession No. Hs 505625(C12orf10), Accession No. Hs 505652(COPZ1), Accession No. Hs 505676(CIP29), Accession No. Hs 505705(MYL6), Accession No. Hs 505806(PBXIP1), Accession No. Hs 505824(CGI-51), Accession No. Hs 506215(RARS), Accession No. Hs 506325(NUDT4), Accession No. Hs 06759(ATP2A2), Accession No. Hs 506861(DDX54), Accession No. Hs 507074(KIAA0152), Accession No. Hs 507162(FLJ12750), Accession No. Hs 507584(MGC9850), Accession No. Hs 507680(PFAAP5), Accession No. Hs 507910(PGRMC2), Accession No. Hs 507916(TGFB1I4), Accession No. Hs 508010(FNDC3A), Accession No. Hs 508644(FLJ10154), Accession No. Hs 509163(KIAA1181), Accession No. Hs 509226(FKBP3), Accession No. Hs 509264(KLHDC2), Accession No. Hs 509414(KTN1), Accession No. Hs 509622(RGL2), Accession No. Hs 509736(HSPCB), Accession No. Hs 509791(ERH), Accession No. Hs 509909(NUMB), Accession No. Hs 510087(ENSA), Accession No. Hs 510328(DDX24), Accession No. Hs 510402(MCP), Accession No. Hs 511067(FLJ10579), Accession No. Hs 511138(DKFZP564G2022), Accession No. Hs 511149(SNAP23), Accession No. Hs 511425(SRP9), Accession No. Hs 511504(TCF12), Accession No. Hs 511862, Accession No. Hs 511952(CBX6), Accession No. Hs 512005(ARPC3), Accession No. Hs 512465(SURF4), Accession No. Hs S12525(RPS17), Accession No. Hs 512607(MIR16), Accession No. Hs 512640(PRKCSH), Accession No. Hs 512661(KIAA1160), Accession No. Hs 512676, Accession No. Hs 512693(FLJ20859), Accession No. Hs 512756(THAP7), Accession No. Hs 512815(AP3D1), Accession No. Hs 512857(CD151), Accession No. Hs 512867(1163), Accession No. Hs 512908(ARPP-19), Accession No. Hs 513043(C15orf12), Accession No. Hs 513055(REC14), Accession No. Hs 513057(RANBP5), Accession No. Hs 513058(TMED3), Accession No. Hs 513071(MESDC1), Accession No. Hs 513083(RPL9), Accession No. Hs 513141(IDH2), Accession No. Hs 513145(NEUGRIN), Accession No. Hs 513153(FURIN), Accession No. Hs 513230(MRPL28), Accession No. Hs 513242(RHOT2), Accession No. Hs 513261(C16orf34), Accession No. Hs 513266(NDUFB10), Accession No. Hs 513470(NFATC2IP), Accession No. Hs 513488(MVP), Accession No. Hs 513490(ALDOA), Accession No. Hs 513520(BCKDK), Accession No. Hs 513522(FUS), Accession No. Hs 513631(ARL2BP), Accession No. Hs 513856(DPH2L1), Accession No. Hs 513984(FLII), Accession No. Hs 514012(MAP2K3), Accession No. Hs 514036(SDF2), Accession No. Hs 514038(FLOT2), Accession No. Hs 514174(JUP), Accession No. Hs 514196(RPL27), Accession No. Hs 514211(MGC4251), Accession No. Hs 514216(CGI-69), Accession No. Hs 514220(GRN), Accession No. Hs 514297(FLJ13855), Accession No. Hs 514303(PHB), Accession No. Hs 514435(SF3B3), Accession No. Hs 514489(WBP2), Accession No. Hs 514535(LGALS3BP), Accession No. Hs 514581(ACTG1), Accession No. Hs 514590(HGS), Accession No. Hs 514819(AP2B1), Accession No. Hs 514870(ATP5F1), Accession No. Hs 514920(NDP52), Accession No. Hs 514934(CAPZA1), Accession No. Hs 515003(C19orf6), Accession No. Hs 515005(STK11), Accession No. Hs 515018(GNA13), Accession No. Hs 515053(AES), Accession No. Hs 515070(EEF2), Accession No. Hs 515092(CLPP), Accession No. Hs 515155(MGC2803), Accession No. Hs 515162(CALR), Accession No. Hs 515164(GADD45GIP1), Accession No. Hs 515210(DNAJB1), Accession No. Hs 515255(LSM4), Accession No. Hs 515266(RENT1), Accession No. Hs 515271(SFRS14), Accession No. Hs 515329(RPL22), Accession No. Hs 515371(CAPNS1), Accession No. Hs 515406(AKT2), Accession No. Hs 515417(EGLN2), Accession No. Hs 515432(DEDD2), Accession No. Hs 515472(SNRPD2), Accession No. Hs 515475(SYMPK), Accession No. Hs 515487(CALM3), Accession No. Hs 515494(SLC1A5), Accession No. Hs 515500(SAE1), Accession No. Hs 515515(KDELR1), Accession No. Hs 515517(RPL18), Accession No. Hs 515524(NUCB1), Accession No. Hs 515540(PTOV1), Accession No. Hs 515550(LOC284361), Accession No. Hs 515598(PRPF31), Accession No. Hs 515607(PPP1R12C). Accession No. Hs 515642(GPSN2), Accession No. Hs 515785(BLVRB), Accession No. Hs 515846(RUVBL2), Accession No. Hs 515848(HADHB), Accession No. Hs 515890(YPEL5), Accession No. Hs 516075(TIA1), Accession No. Hs 516077(FLJ14668), Accession No. Hs 516087(TEX261), Accession No. Hs 516111(DCTN1), Accession No. Hs 516114(WBP1), Accession No. Hs 516157(MAT2A), Accession No. Hs 516450(FLJ20297), Accession No. Hs 516522(FLJ21919), Accession No. Hs 516539(HNRPA3), Accession No. Hs 516587(UBE2Q), Accession No. Hs 516633(NCKAP1), Accession No. Hs 516711(CHPF), Accession No. Hs 516790(ARHGEF2), Accession No. Hs 516807(STK25), Accession No. Hs 516826(TRIB3), Accession No. Hs 516855(CENPB), Accession No. Hs 517080(SLC35C2), Accession No. Hs 517106(CEBPB), Accession No. Hs 517134(C20orf43), Accession No. Hs 517145(ENO1), Accession No. Hs 57168(TAGLN2), Accession No. Hs 517216(PEA15), Accession No. Hs 517232(PEX19), Accession No. Hs 517240(IFNGR2), Accession No. Hs 517262(SON), Accession No. Hs 517293(F11R), Accession No. Hs 517338(ATP6V1E1), Accession No. Hs 17342(DEDD), Accession No. Hs 517356(COL18A1), Accession No. Hs 517357(DGCR2), Accession No. Hs 517421(PCQAP), Accession No. Hs 517438(ASCC2), Accession No. Hs 517517(EP300), Accession No. Hs 517543(PES1), Accession No. Hs 517582(MCM5), Accession No. Hs 517622(UNC84B), Accession No. Hs 517641(L3MBTL2), Accession No. Hs 517666(DIA1), Accession No. Hs 517731(PP2447), Accession No. Hs 517768(DKFZP564B167), Accession No. Hs 517792(C3orf10), Accession No. Hs 517817(MGC3222), Accession No. Hs 517821, Accession No. Hs 517888(CRTAP), Accession No. Hs 517948(DHX30), Accession No. Hs 517949(MAP4), Accession No. Hs 517969(APEH), Accession No. Hs 517981(TUSC2), Accession No. Hs 518060(ARL6IP5), Accession No. Hs 518123(TFG), Accession No. Hs 518236(SEC61A1), Accession No. Hs 518244(RPN1), Accession No. Hs 518249(ZNF9), Accession No. Hs 518250(COPG), Accession No. Hs 518265(H41). Accession No. Hs 518326(SERP1), Accession No. Hs 518346(SSR3), Accession No. Hs 518374(QSCN6), Accession No. Hs 518424(NDUFB5), Accession No. Hs 518460(AP2M1), Accession No. Hs 518464(PSMD2), Accession No. Hs 518525(GLUL), Accession No. Hs 518551(RPL31), Accession No. Hs 518608(PP784), Accession No. Hs 518609(ARPC5), Accession No. Hs 518750(OCIAD1), Accession No. Hs 18805(HMGA1), Accession No. Hs 518827(CCNI), Accession No. Hs 519276(MAPKAPK2), Accession No. Hs 519304(PELO), Accession No. Hs 519346(ERBB2IP), Accession No. Hs 519347(SFRS12), Accession No. Hs 519520(RPS25), Accession No. Hs 519523(SERPINB6), Accession No. Hs 519557(TMEM14C), Accession No. Hs 519718(TTC1), Accession No. Hs 519756(STK10), Accession No. Hs 519818(MGAT1), Accession No. Hs 519909(MARCKS), Accession No. Hs 519930(C6orf62), Accession No. Hs 520026(VARS2), Accession No. Hs 520028(HSPA1A), Accession No. Hs 520037(NEU1), Accession No. Hs 520070(C6orf82), Accession No. Hs 520140(SRF), Accession No. Hs 520189(ELOVL5), Accession No. Hs 520205(EIF2AK1), Accession No. Hs 520210(KDELR2), Accession No. Hs 520287(C6orf111), Accession No. Hs 520313(CD164), Accession No. Hs 520383(STX7), Accession No. Hs 520421(PERP), Accession No. Hs 520459(GTF21), Accession No. Hs 520623(C7orf27), Accession No. Hs 520640(ACTB), Accession No. Hs 520740(SCRN1), Accession No. Hs 520794(YKT6), Accession No. Hs 520898(CTSB), Accession No. Hs 520943(WBSCR1), Accession No. Hs 520967(MDH2), Accession No. Hs 520973(HSPB1), Accession No. Hs 520974(YWHAG), Accession No. Hs 521064(ZNF655), Accession No. Hs 521151(FLJ22301), Accession No. Hs 521289(REPIN1), Accession No. Hs 521487(MGC8721), Accession No. Hs 521640(RAD23B), Accession No. Hs 521809(COBRA1), Accession No. Hs 521903(LY6E), Accession No. Hs 521924(SIAHBPI), Accession No. Hs 521969(NDUFB11), Accession No. Hs 521973(WDR13), Accession No. Hs 522074(DSIPI), Accession No. Hs 522110(CREB3), Accession No. Hs 522114(CLTA), Accession No. Hs 522310(NANS), Accession No. Hs 522373(GSN), Accession No. Hs 522394(HSPA5), Accession No. Hs 522463(EEF1A1), Accession No. Hs 522507(FBXW5), Accession No. Hs 522584(TMSB4X), Accession No. Hs 522590(EIF1AX), Accession No. Hs 522632(TIMP1), Accession No. Hs 522665(MAGED2), Accession No. Hs 522675(FLJ12525), Accession No. Hs 522752(PSMD10), Accession No. Hs 522817(BCAP31), Accession No. Hs 522819(IRAK1), Accession No. Hs 522823(EMD), Accession No. Hs 522932(NCOA4), Accession No. Hs 522995(EIF4EBP2), Accession No. Hs 523004(PSAP), Accession No. Hs 523012(DDIT4), Accession No. Hs 523054(SMP1), Accession No. Hs 523131(TRAPPC3), Accession No. Hs 523145(DDOST), Accession No. Hs 523215(NDUFB8), Accession No. Hs 523238(NOLC1), Accession No. Hs 523262(C1orf8), Accession No. Hs 523299(EIF3S10), Accession No. Hs 523302(PRDX3), Accession No. Hs 523560(HSPCA), Accession No. Hs 523680(SSRP1), Accession No. Hs 523789(TncRNA), Accession No. Hs 523829(POLD4), Accession No. Hs 523836(GSTP1), Accession No. Hs 523852(CCND1), Accession No. Hs 523875(INPPL1), Accession No. Hs 524009(AASDHPPT), Accession No. Hs 524081(DPAGT1), Accession No. Hs 524084(RNF26), Accession No. Hs 524161(RSU1), Accession No. Hs 524171(RAD52), Accession No. Hs 524183(FKBP4), Accession No. Hs 524195(ARHGAP21), Accession No. Hs 524214(MLF2), Accession No. Hs 524219(TPI1), Accession No. Hs 524271(PHC2), Accession No. Hs 524367(CBARA1), Accession No. Hs 524395(TUBA3), Accession No. Hs 524464(ATP5G2), Accession No. Hs 524502(RNF41), Accession No. Hs 524530(CTDSP2), Accession No. Hs 524590(RAB21), Accession No. Hs 524599(NAP1L1), Accession No. Hs 524690(PPIE), Accession No. Hs 524788(RAB35), Accession No. Hs 524809(RSN), Accession No. Hs 524899(SAP18), Accession No. Hs 524920(ZFP91), Accession No. Hs 524969(Ufm1), Accession No. Hs 525134(FLJ20277), Accession No. Hs 525163(ANKRD10), Accession No. Hs 525232(LRP10), Accession No. Hs 525238(C14orf119), Accession No. Hs 525330(ARF6), Accession No. Hs 525391(FLJ20580), Accession No. Hs 525527(RER1), Accession No. Hs 525626(PACS1L), Accession No. Hs 525899(C6orf49), Accession No. Hs 526464(PML), Accession No. Hs 526521(MDH1), Accession No. Hs 527105(HNRPDL), Accession No. Hs 527193(RPS23), Accession No. Hs 527348(AKAP9), Accession No. Hs 527412(ASAH1), Accession No. Hs 527861(OS-9), Accession No. Hs 527862(PKD1), Accession No. Hs 527980(DUT), Accession No. Hs 528050(HARS), Accession No. Hs 528222(NDUFS4), Accession No. Hs 528300(PITRM1), Accession No. Hs 528305(DDX17), Accession No. Hs 528572(SCAM-1), Accession No. Hs 528668(RPL6), Accession No. Hs 528780(GSPT1), Accession No. Hs 528803(UQCRC2), Accession No. Hs 529059(EIF3S4), Accession No. Hs 529132(SEPW1), Accession No. Hs 529244(NCK2), Accession No. Hs 529280(ANAPC7), Accession No. Hs 529303(ARPC2), Accession No. Hs 529369(AFAP), Accession No. Hs 529400(IFNAR1), Accession No. Hs 529420(UBE2G2), Accession No. Hs 529591(TLOC1), Accession No. Hs 529618(TFRC), Accession No. Hs 529631(RPL35A), Accession No. Hs 529782(VCP), Accession No. Hs 529798(BTF3), Accession No. Hs 529862(CSNK1A1), Accession No. Hs 529890(CANX), Accession No. Hs 529892(SQSTM1), Accession No. Hs 529957(SEC63), Accession No. Hs 530096(EIF3S2), Accession No. Hs 530118(LUC7L2), Accession No. Hs 530291(ANXA11), Accession No. Hs 530314(SSNA1), Accession No. Hs 530331(PDHA1), Accession No. Hs 530381(PIM3), Accession No. Hs 530412(PAI-RBP1), Accession No. Hs 530436(STXBP3), Accession No. Hs 530479(PMF1), Accession No. Hs 530687(RNH), Accession No. Hs 530734(MRPL16), Accession No. Hs 530753(FLJ20625), Accession No. Hs 530823(COPS7A), Accession No. Hs 530862(PRKAG1), Accession No, Hs 531081(LGALS3), Accession No. Hs 531089(PSMA3), Accession No. Hs 531176(SARS), Accession No. Hs 531330(CBWD1), Accession No. Hs 531614(BTBD14B), Accession No. Hs 531752(RANBP3), Accession No. Hs 531856(GAS5), Accession No. Hs 531876(DNCL2A), Accession No. Hs 531879(RAD1), Accession No. Hs 532359(RPL5), Accession No. Hs 532399(KIAA0663), Accession No. Hs 532755(GTL3), Accession No. Hs 532790(NMT1), Accession No. Hs 532793(KPNB1), Accession No. Hs 532803(HN1), Accession No. Hs 532826(MCL1), Accession No. Hs 532853(NDUFB7), Accession No. Hs 533030(HRIHFB2122), Accession No. Hs 533059(TUBB), Accession No. Hs 533122(SFRS10), Accession No. Hs 533136(LRPAP1), Accession No. Hs 533192(TOMM20), Accession No. Hs 533222(HSA9761), Accession No. Hs 533245(DDX46), Accession No. Hs 533282(NONO), Accession No. Hs 533308(PPP2R5D), Accession No. Hs 533317(VIM), Accession No. Hs 533437(TCEB1), Accession No. Hs 533440(WWP1), Accession No. Hs 533474(PPP1R8), Accession No. Hs 533479(LYPLA2), Accession No. Hs 533526(ATRX), Accession No. Hs 533624(H3F3A), Accession No. Hs 533712(RBM4), Accession No. Hs 533732(SRP14), Accession No. Hs 533771(STUB1), Accession No. Hs 533782(KRT8), Accession No. Hs 533977(TXNIP), Accession No. Hs 533985(EXOC7), Accession No. Hs 533986(ZNF258), Accession No. Hs 534125(HLA-C), Accession No. Hs 534168(NDUFA1), Accession No. Hs 534212(SEC22L1), Accession No. Hs 534255(B2M), Accession No. Hs 534307(CCND3), Accession No. Hs 534314(EIF5A), Accession No. Hs 534326(ITGB48P), Accession No. Hs 534338(PPP4C), Accession No. Hs 534346(RPS7), Accession No. Hs 534350(SMARCB1), Accession No. Hs 534453(GRIM19), Accession No. Hs 534456(ANAPC11), Accession No. Hs 534457(C14orf166), Accession No. Hs 534473(TOMM22), Accession No. Hs 534483(MGC2941), Accession No. Hs 536275(PACS1), Accession No. Hs 541269(NDUFB9), Accession No. Hs 546248(CTSD), Accession No. Hs 546250(DNCI2), Accession No. Hs 546253(FDFT1), Accession No. Hs 546261(HNRPA1), Accession No. Hs 546269(RPL10A), Accession No. Hs 546271(PCBP2), Accession No. Hs 546286(RPS3), Accession No. Hs 546289(RPS12), Accession No. Hs 546290(RPS18), Accession No. Hs 546291(NET-5), Accession No. Hs 546339(SMAP), Accession No. Hs 546356(RPL13A), Accession No. Hs 546394(HSPC016), Accession No. Hs 547759(SSBP3), Accession No. Hs 549178(C9orf86), Accession No. Hs 552590(HTF9C), Accession No. Hs 553496(PGM3), Accession No. Hs 553512(C3F), Accession No. Hs 554767(NUP88), Accession No. Hs 554776(SREBF1), Accession No. Hs 554894(FLJ12953), Accession No. Hs 554896(MGC11257), Accession No. Hs 555194(FAM36A), Accession No. Hs 555866(C1QBP), Accession No. Hs 555873(HNRPAB), Accession No. Hs 555875(IDH3A), Accession No. Hs 555889(PSMC2), Accession No. Hs 555890(RBBP4), Accession No. Hs 555911 (RBM8A), Accession No. Hs 555969(RIC8), Accession No. Hs 555971(PP1201), Accession No. Hs 555973(MRPS25), Accession No. Hs 555994(LONP), Accession No. Hs 556267(FBXL10), Accession No. Hs 556461(NDUFV2), Accession No. Hs 556795(PAICS), Accession No. Hs 557550(NPM1), Accession No. Hs 558296(ACP1), Accession No. Hs 558313(COX6A1), Accession No. Hs 558322(EEF1B2), Accession No. Hs 558325(EIF5), Accession No. Hs 558328(FKBP5), Accession No. Hs 558330(FTL), Accession No. Hs 558338(HSPE1), Accession No. Hs 558345(IK), Accession No. Hs 558354(LAMR1), Accession No. Hs 558360(NDUFB4), Accession No. Hs 558361(NME2), Accession No. Hs 558362(NUMA1), Accession No. Hs 558376(RAC1), Accession No. Hs 558381(RNU65), Accession No. Hs 558382(RPL15), Accession No. Hs 558383(RPL18A), Accession No. Hs 558384(RPL19), Accession No. Hs 558385(RPL23A), Accession No. Hs 558386(RPL34), Accession No. Hs 558388(RPS3A), Accession No. Hs 558389(RPS8), Accession No. Hs 558390(RPS24), Accession No. Hs 558391(RPS26), Accession No. Hs 558396(SCD), Accession No. Hs 558424(CSDA), Accession No. Hs 558426(EIF3SS), Accession No. Hs 558429(G10), Accession No. Hs 558431(RPL14), Accession No. Hs 558442(PDCD6IP), Accession No. Hs 558448(TXNL2), Accession No. Hs 558453(ATP5L), Accession No. Hs 558454(NUDC), Accession No. Hs 558458(COPS8), Accession No. Hs 558473(C18orf10), Accession No. Hs 558499(8D6A), Accession No. Hs 558511(PSARL), Accession No. Hs 558521(C2orf33), Accession No. Hs 558591(ORMDL1), Accession No. Hs 558825(PDE4DIP), Accession No. Hs 558995, Accession No. Hs 567260(CD2BP2), Accession No. Hs 567263(C1orf43), Accession No. Hs 567267 (ATP2C1) and Hs 567279 (HCNGP). In addition, the selected gene is characterized to be a gene selected from the group consisting of AP3B1, IPO8, PLP0, EIF2C1, SFRS9, and UBC used for calibration, and RNaseP, TERT, LINE-1, MTHFR, RPPH1, BB, and PPIH used as Internal Amplification Control (IAC). The selected gene is characterized to be a primer pair and/or probe capable of specifically amplifying the candidate endogenous reference gene.

### Example 2: Verification of Reference Genes for CNV Detection of c-Met Gene

With respect to a total of 8 reference genes established for CNV detection of a c-Met gene, integrity (suitability as an actual reference) investigation was performed using The Cancer Genome Atlas (TCGA) database. At this time, Ribonuclease P (RNase P) and Long Interspersed Element-1 (LINE-1) which were not included in the TCGA database were excluded from the analysis.

In more detail, the presence of deep deletion, truncating mutation, and missense mutation was analyzed.

As a result of the analysis, it was confirmed that except for telomerase reverse transcriptase (TERT) and canine beta-glucuronidase gene (GUSB), the remaining genes were stably expressed in 11 representative cancer types. Information on reference genes selected based thereon is as follows.

Reference gene 1 is a reference gene extracted and selected from known Document 1 (PLOS ONE DOI: 10.1371/journal.pone.0146784 Jan. 14, 2016), a gene used for gene copy number variation. The reference gene is also known as a reference gene suitable for an experimental technique for the kit according to the present invention.

Reference gene 2 is a reference gene extracted and selected from known Document 2 (British Journal of Cancer (2012) 107, 325-333) and is disclosed in the internal amplification control (IAC) list. The reference gene is also known as a reference gene suitable for the experimental technique for the kit according to the present invention.

Reference gene 3 is a reference gene extracted and selected from known Document 3 (Annals of Oncology 28: 1508-1516, 2017), and is known as a reference gene suitable for the experimental technique for the kit according to the present invention. The Reference gene 3 is the same gene as Reference gene 5, and has only a different type.

Reference gene 4 is a gene selected from known Document 4 (Cancer Sci 105 (2014) 1032-1039) and is disclosed in the IAC list like Reference gene 2.

Reference gene 6 was selected as one of a housekeeping gene candidate group disclosed in known Document 5(BMC Molecular Biology. 2009) and located in the same chromosome as c-Met. It is also known that the gene is used for normalization.

### Example 3: Re-Verification of Reference Genes for CNV Detection of c-Met Gene

CNV observation using each reference gene was verified through cell line experiments and lateral organ boundary (LOB) and limit of detection (LOD) experiments using reference materials.

For normalization, among eight reference genes, three of six reference genes were mixed, respectively, excluding TERT, an unstable reference gene identified through TCGA database analysis, and GUSB, a gene identified through documentary survey, and a mean thereof was calculated. The reference genes were grouped based on an amplitude value for each reference to be selected into two groups (Table 2).

TABLE 2

| Gene | Name | Amplitude value |
|---|---|---|
| Reference gene 1 | RnaseP | 3500 to 4000 |
| Reference gene 2 | PPIA | 4000 to 4500 |
| Reference gene 3 | AP3B1 Type1 | 4000 to 4500 |
| Reference gene 4 | RPP30 | 2500 to 3000 |
| Reference gene 5 | AP3B1 Type2 | 4000 to 4500 |
| Reference gene 6 | EIF2C1 | 4000 to 4500 |

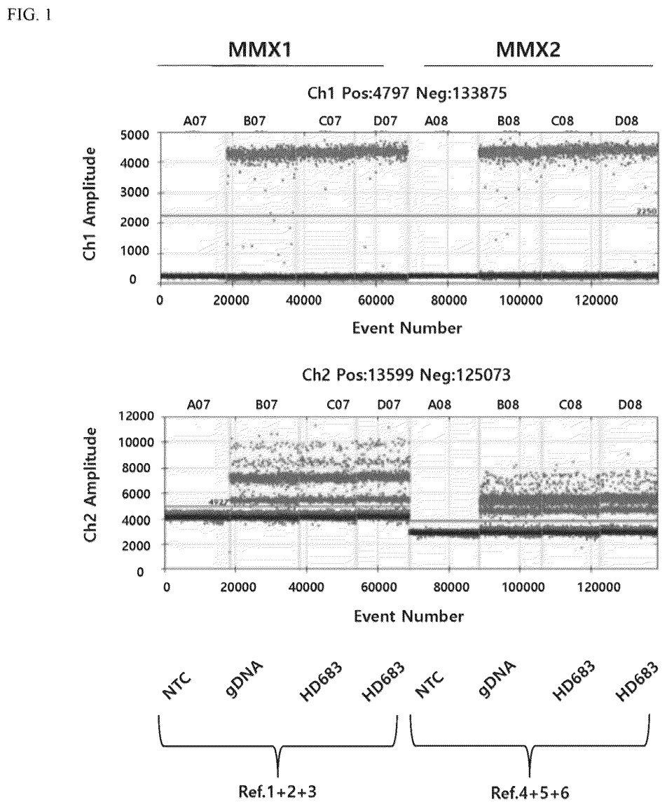

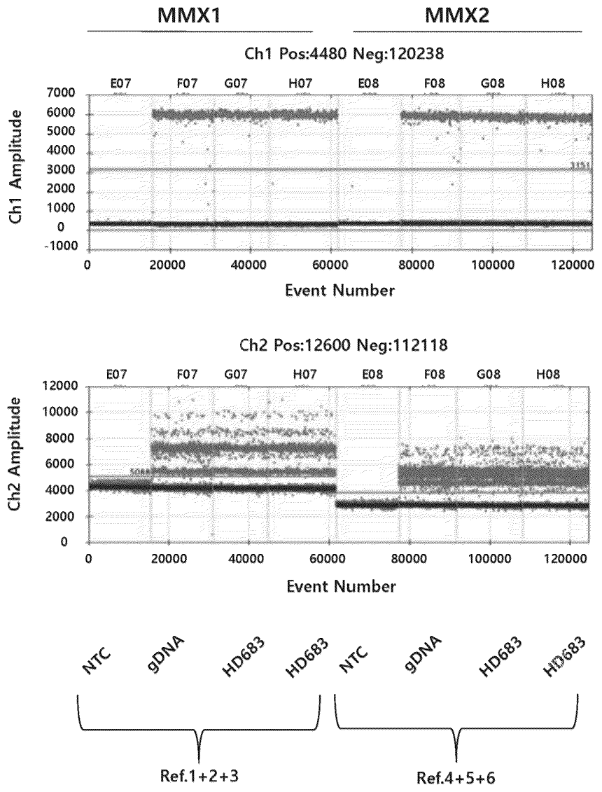

As a result of performing experiments on Met target exons 2 and 15 with respect to the selected two groups, as illustrated in FIG. 1, when the reference was mixed, as a probe baseline was raised, it was difficult to be distinguished from a target signal of a 5′Hexachloro-Fluorescein (HEX) channel.

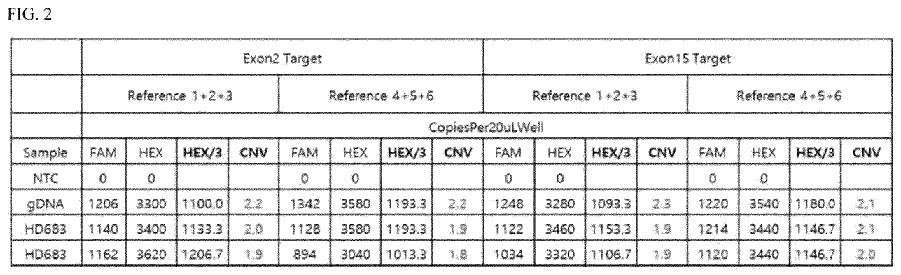

Accordingly, after setting the threshold and analyzing the copy number, CNV was calculated for a mean value (ref mix/3) calculated for each reference group, and as a result, as illustrated in FIG. 2, it was confirmed that the CNV was close to CNV 2 fold.

In addition, in order to increase the degree of separation between a probe background and a target, a re-experiment was performed by reducing a probe concentration of the three mixtures to ½, but it was confirmed that the separation tended to be less than the result of FIG. 1. In addition, the experiment was conducted again by mixing two for six reference genes, but it was not separated from the baseline, and the copy number of the reference genes was also derived at half level.

Therefore, a method of normalizing the genes by mixing the reference genes was determined as an undesirable method.

### Example 4: Determination of Panel for CNV Detection of c-Met Gene

A stable panel capable of CNV detection of a c-MET gene was completed through a reference gene mixing experiment and an interference reaction (Table 3, the panel named MMX). Five reference genes were selected and a target c-MET was determined as exon15. At this time, when exon2 was targeted for c-MET, there was no difference from exon15 in the experimental result, but exon15 was selected because exon15 showed a high amplitude value. At this time, probes and primers of Tables 4 and 5 below were used for CNV detection.

TABLE 3

| Panel | Detection | Channel |
|---|---|---|
| MMX 1 | c-Met Target (ch. 7) | FAM |
| | Reference gene 1 | HEX |
| MMX 2 | Reference gene 2 | FAM |
| | Reference gene 3 | HEX |
| MMX 3 | Reference gene 4 | FAM |
| | Reference gene 5 (ch. 7) | HEX |

TABLE 4

| | | | | SEQ |
|---|---|---|---|---|
| | Gene | Probe | | ID |
| Panel | (exon) | name | Sequence | NO: |
| MMX | c-Met | c-Met Target | CCATCCTAACTAGTG | 1 |
| 1 | exon 15 + | (ch.7) | GGGACTCTGA | |
| | Reference | Reference | CACAGTTTGTCAGCC | 2 |
| | | gene 1 | CTTGGA | |
| MMX | Reference | Reference | ACGGCCAGCGAAGT | 3 |
| 2 | | gene 2 | GAGTTC | |
| | | Reference | TCCAACCTCTGCCTC | 4 |
| | | gene 3 | AAGATC | |
| MMX | Reference | Reference | GGAATTGTCAAACTG | 5 |
| 3 | | gene 4 | ACTCCTTTTCC | |
| | | Reference | TTGGATGGCAAGCAT | 6 |
| | | gene 5 | GTGGT | |
| | | (ch.7) | | |

TABLE 5

| | | | | SEQ |
|---|---|---|---|---|
| | Gene | Primer | | ID |
| Panel | (exon) | name | Sequence | NO: |
| MMX | c-Met exon | c-Met | AAGTGCAGTATCCT | 7 |
| 1 | 15 + | Target F1 | CTGACAGACA | |
| | Reference | c-Met | TGGACAGTATTTTGC | 8 |
| | | Target R1 | AGTAATGG | |
| | | Reference | CCACATGCTAACTC | 9 |
| | | gene 1 F1 | GATATGC | |
| | | Reference | GTGGAAGACTGTTC | 10 |
| | | gene 1 R1 | AACACTTACC | |
| MMX | Reference | Reference | TGCCGGAGCTTGGA | 11 |
| 2 | | gene 2 F1 | ACAG | |
| | | Reference | AAAATGGGCGGAGG | 12 |
| | | gene 2 R1 | AGAGTAG | |
| | | Reference | CAGTGTGTGCAGGT | 13 |
| | | gene 3 F3 | GAAGAA | |
| | | Reference | TGTTAATGCCACCA | 14 |
| | | gene 3 R2 | AGTTTGAC | |
| MMX | Reference | Reference | ATTTTAGGATGTTTT | 15 |
| 3 | | gene 4 F1 | TCGCATCTG | |
| | | Reference | TGGCAGAATTTGTT | 16 |
| | | gene 4 R1 | CCTATGC | |
| | | Reference | GTTGCCAGTCATAGT | 17 |
| | | gene 5 F3 | GATTGTTC | |
| | | Reference | GGCCTCCACAATATT | 18 |
| | | gene 5 R3 | CATGC | |

CNV normalization was calculated based on a mean copy number of five reference genes (Equation 1). At this time, if CNV was 2 or more, it was determined that there was CNV.

Through the CNV calculation value through the copy mean value of a plurality of verified reference genes, it is possible to calculate a CNV value with better reliability than the reliability of the CNV calculation value calculated for one basic gene. In addition, it was determined that the reliability of previously known reference genes could be minimized due to the combination of reference genes with different chromosomal positions.

$$\frac{c - Met target}{Average of Ref 1 ∼ 5} \times 2 > 2 \qquad [ Equation 1 ]$$

### Determination Method Using Reference Genes

In the case of

it is determined that CNV is present in c-Met.

In addition, an additional calculation method was introduced to confirm the polysomy of c-Met (Equation 2). When CNV was calculated based on a reference gene (Reference gene 5) located on the same chromosome, it was determined that there was polysomy when the CNV value was close to 2 and when the CNV values of the remaining 4 reference genes were 2 or more.

$$\frac{c - Met target}{Average of Ref S} > 2 \approx 2 and \frac{c - Met target}{Average of Ref 1 ∼ 4} \times 2 > 2 , \qquad [ Equation 2 ]$$

Determination Method Using Reference Genes when Confirming Polysomy

In the case of

it is determined that there is CNV in c-Met.

As a result of an experiment using a panel confirmed from DNAs of three cell lines known to have c-MET CNV, DNA was extracted from each cell pellet using a qiagen DNA Prep kit, and then 3.3 ng of DNA was added and confirmed by a developed ddPCR c-MET CNV kit. A CNV fold value was calculated based on the calculated copy number, and it was confirmed that a positive value was obtained (Table 6).

The three cell lines were gastric cancer cell lines SNU5, HS746T, and MKN45, and were purchased from the Korea Cell Line Bank.

TABLE 6

| Sample | ddMET (CNV fold) |
|---|---|
| gDNA | 2.1 |
| SNU5 | 17.3 |
| HS746T | 22.4 |
| MKN45 | 19.3 |

### Example 5: Verification of Confirmed Panel

Pre-analytical validation was performed on each confirmed MMx panel, and the presence or absence of an interference reaction was first confirmed through cross-reaction. Specimens were identified as reference materials and cell lines.

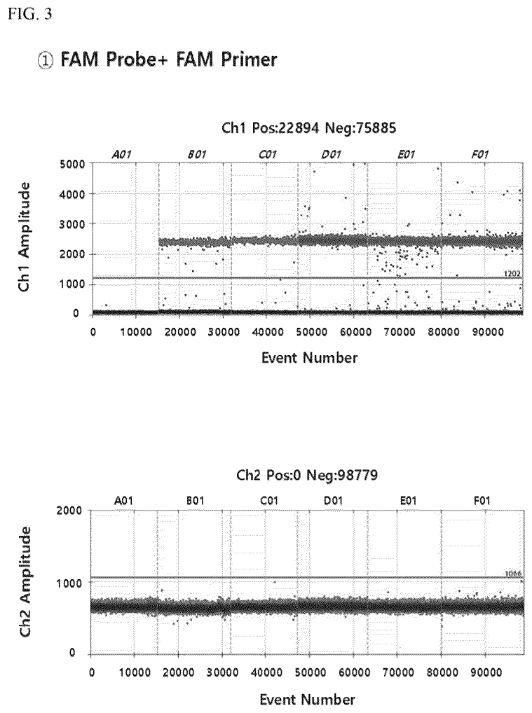

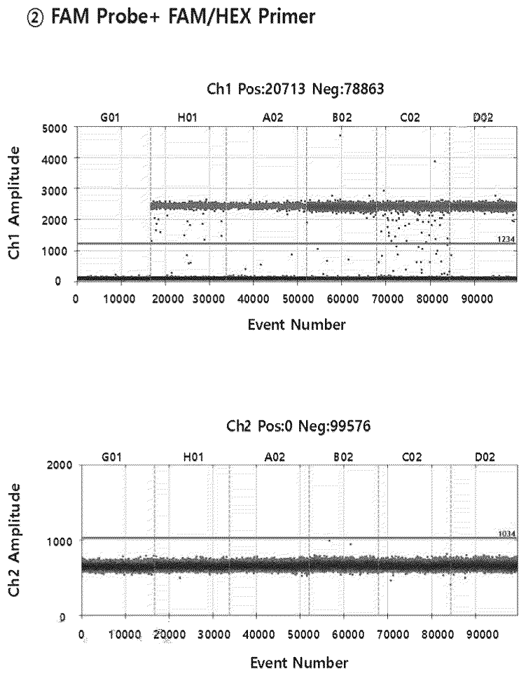

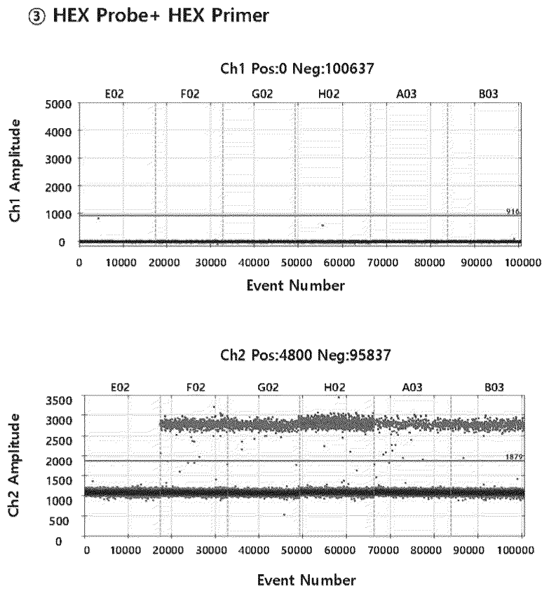

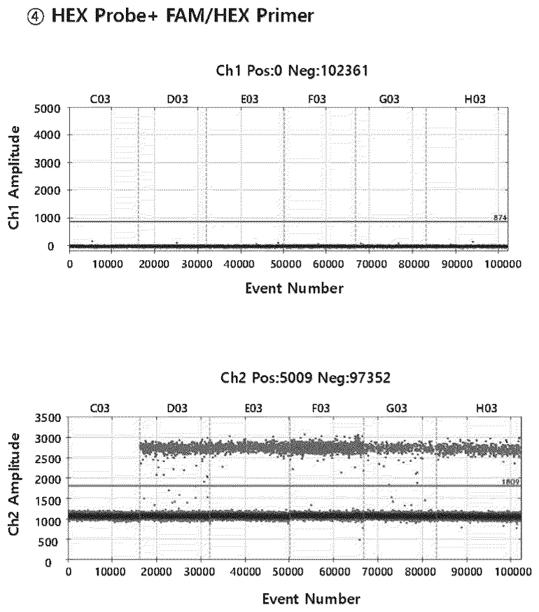

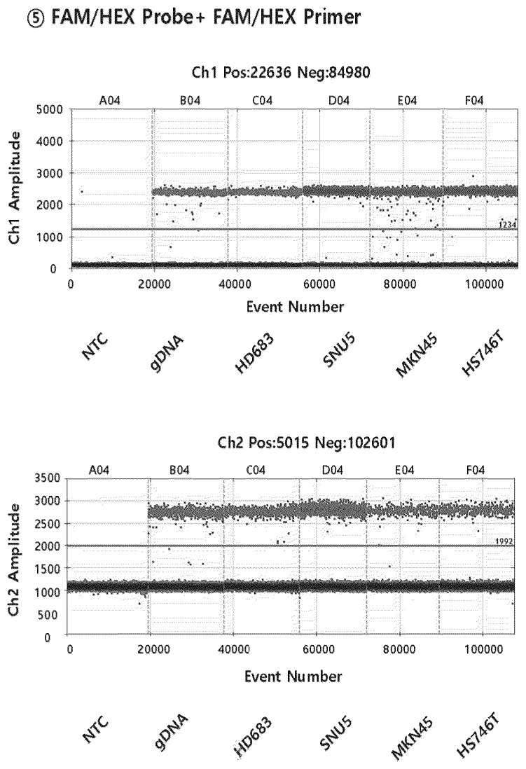

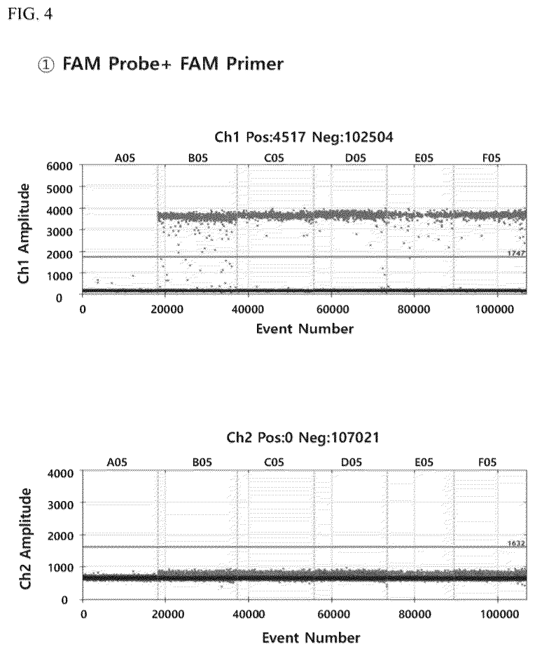

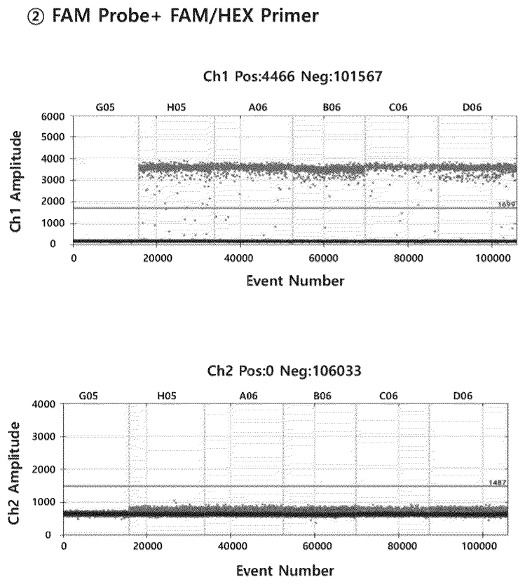

Results for the presence or absence of the interference reaction for each panel were shown in FIGS. 3 to 5.

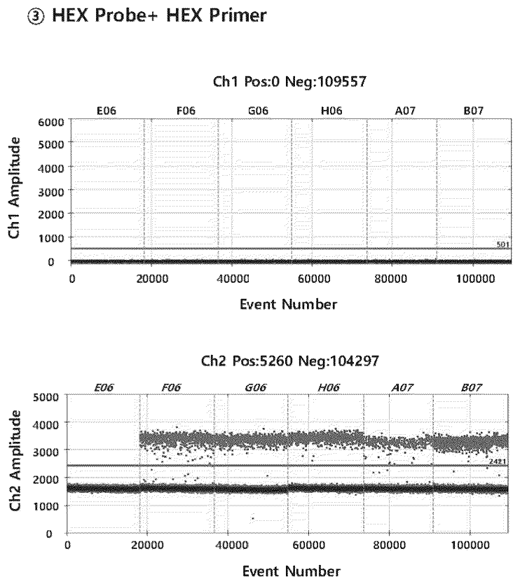

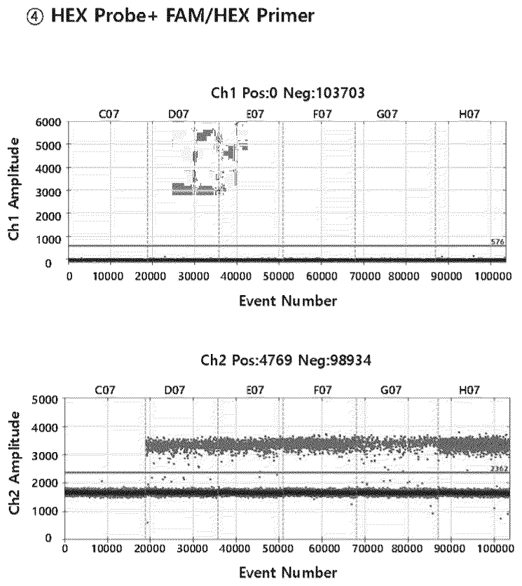

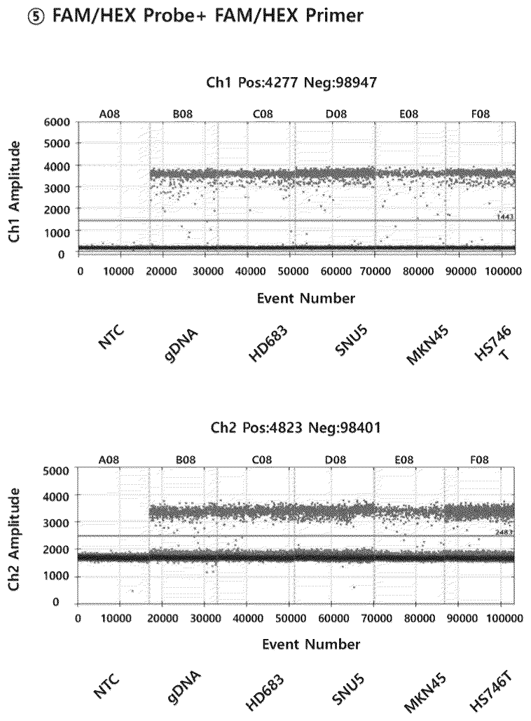

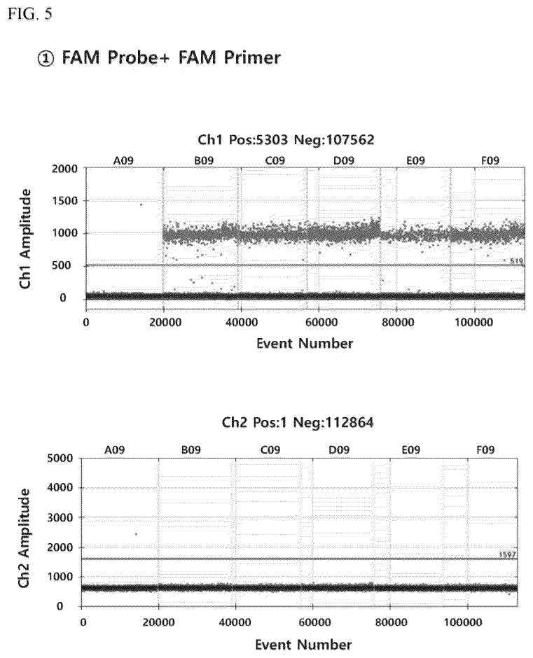

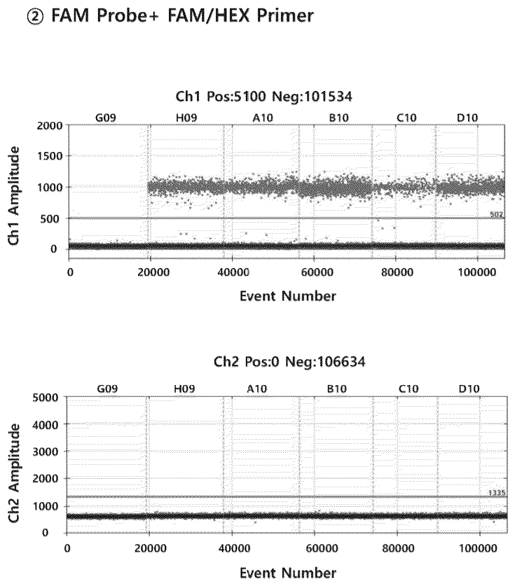

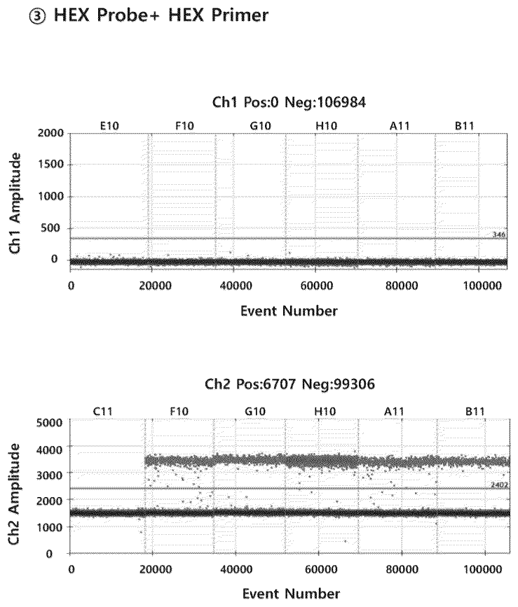

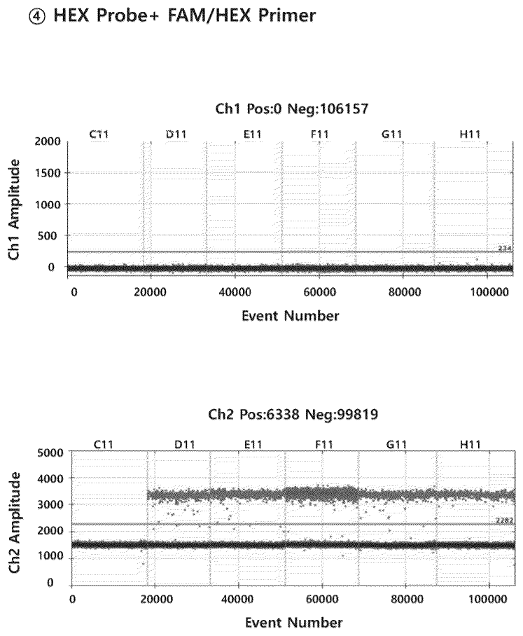

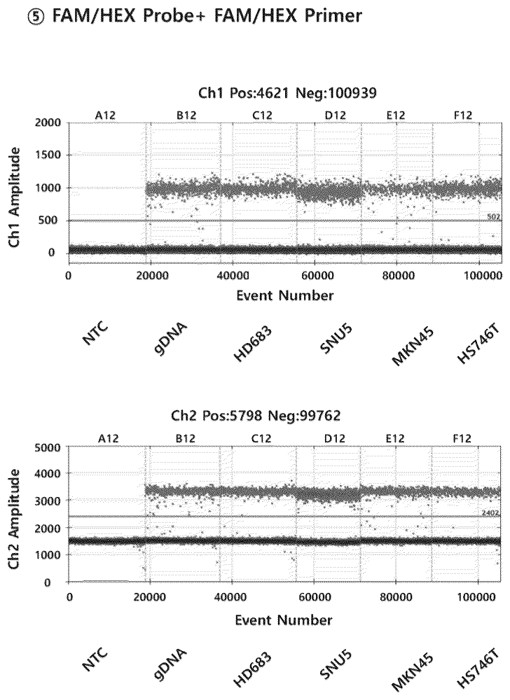

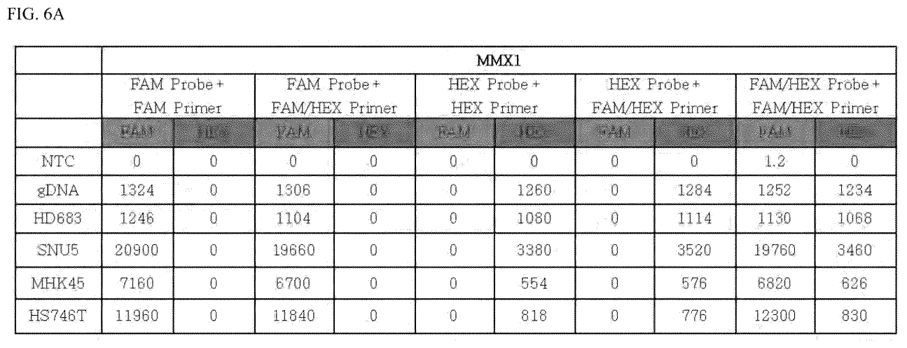

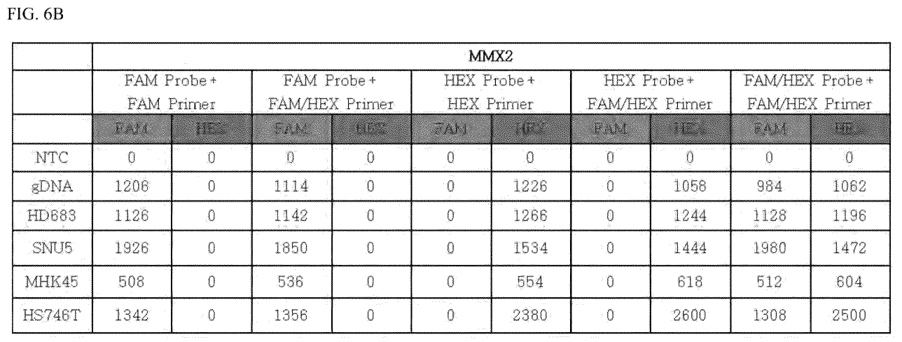

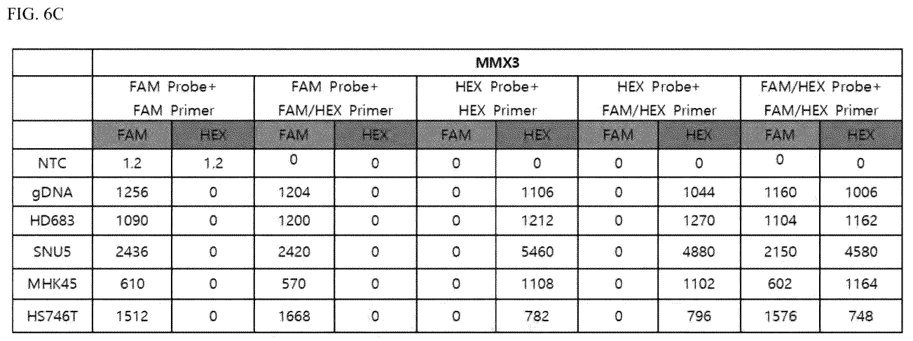

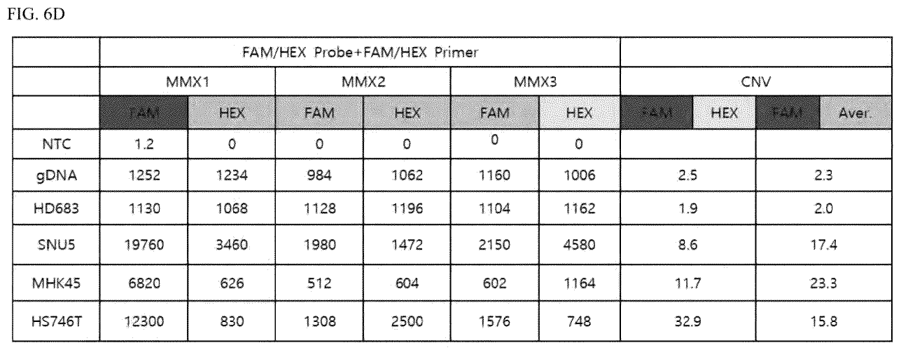

In addition, the copy numbers and CNV values according to the results of the interference reaction experiment for each panel were shown in FIGS. 6A to 6D.

In order to confirm the interference reaction between the primer and the probe for each panel, the interference reaction was confirmed using a reference material HD683 and cell lines SNU5, MKN45, and HS746T known to have CNV as templates.

In addition, it was confirmed whether the FAM probe showed an interference reaction with an HEX primer when the HEX primer was mixed with a primer matched with a FAM probe included in each MMX, and it was confirmed that the HEX probe also showed the interference reaction with the FAM primer. When the copy numbers of Tables in FIGS. 6A, 6B, and 6C were confirmed, it was confirmed that there was no difference in the copy number for each MMX, and thus it was confirmed that the interference did not occur with each other.

In addition, it was confirmed that the CNV values derived from gDNA and the reference material HD683 were stably detected through 10 or more repeated experiments. In addition, even in a normal FFPE sample, similarly, it was confirmed that there was no CNV by detecting the CNV values in 20 FFPE samples, so that it was confirmed that a stable LOB value of CNV was detected in 3.3 ng DNA.

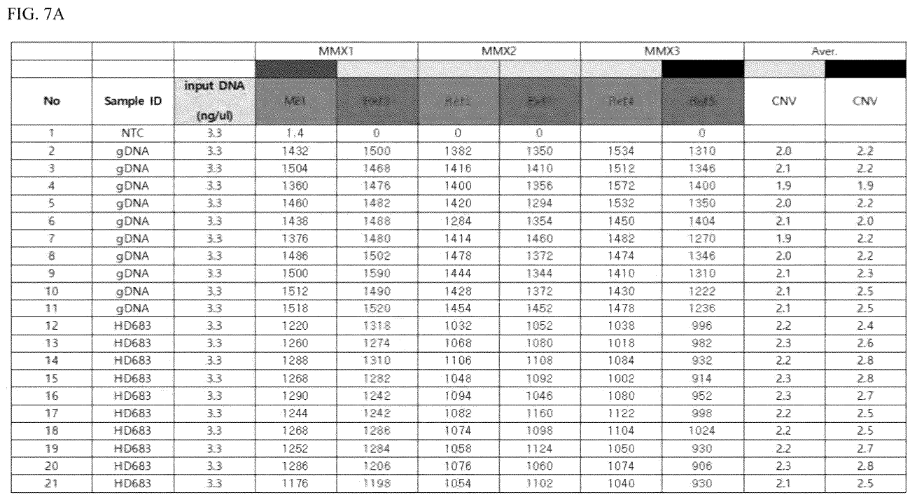

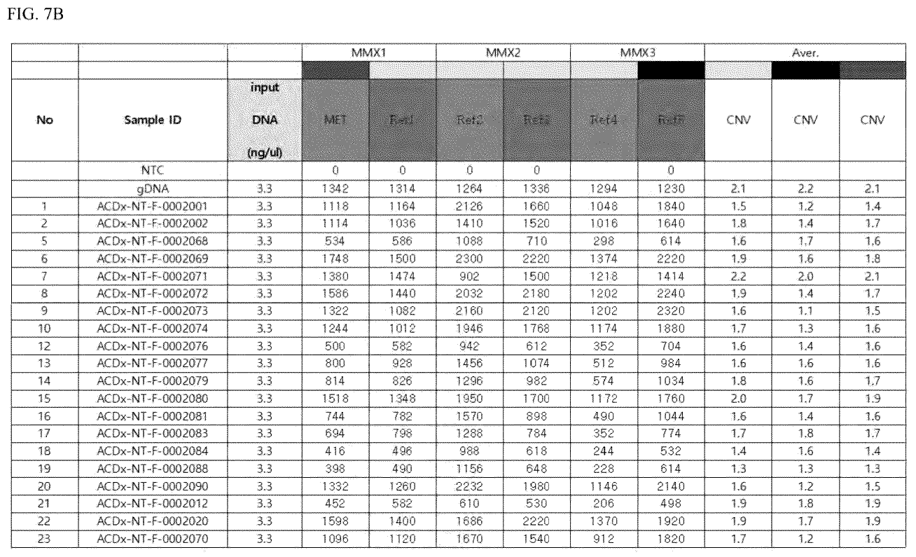

In addition, as a result of 10 or more repeated experiments on a reference material and a known human gDNA sample, as illustrated in FIG. 7A, it was confirmed that a stable LOB value was detected as a CNV value of 2 fold.

In addition, as illustrated in FIG. 7B, it was confirmed that the LOB value was detected as the CNV value of 2 > through a normal FFPE sample (N=20).

An LOD experiment was performed to determine the minimum concentration detected by continuously diluting the input DNA amounts of the human gDNA and the reference material HD683.

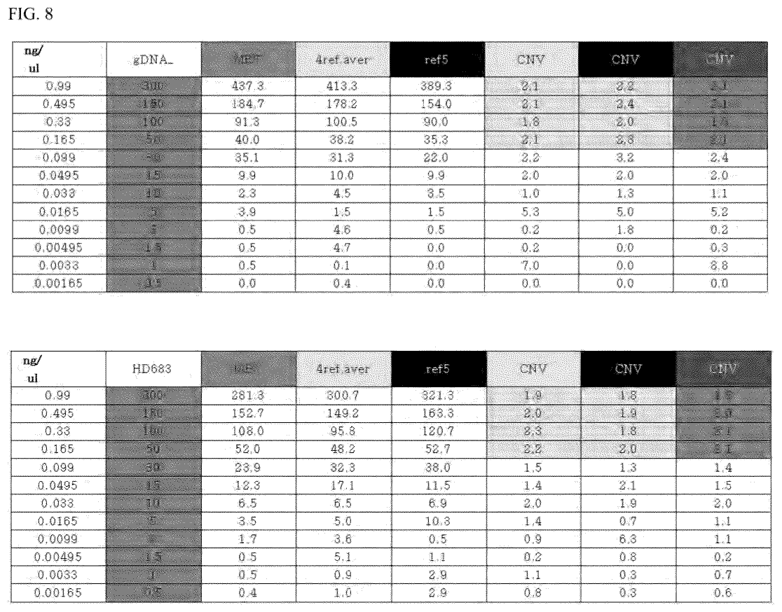

Each DNA concentration was sequentially diluted using the human gDNA and the reference material HD683 to obtain 12 points from 0.99 ng and diluted to 0.00165 ng. After deriving the CNV value from each diluted sample using the developed kit, the degree of fluctuation of the CNV value was checked, and then the minimum detected concentration was confirmed, and the LOD was determined as 0.2 ng. The CNV value was detected by averaging the copy numbers derived through three repeated experiments, and the CNV value was set to 2, and the results were shown in FIG. 8.

CNV values for four reference genes (yellow), CNV values for reference genes located on the same chromosome (black), and CNV values for five reference genes (purple) were detected, and as a result, a minimum concentration LOD that was stably detected was confirmed as 0.2 ng/well. In addition, an R\hat{ }2 value for each copy number was confirmed as 0.98.

### Example 6: Evaluation of Clinical Applicability

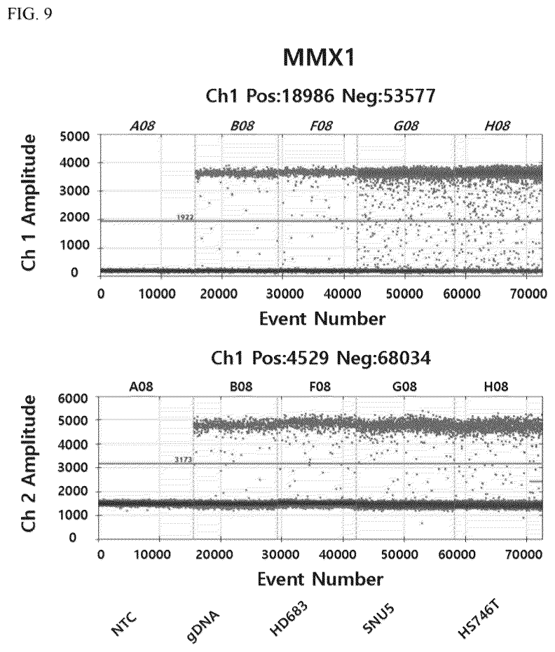

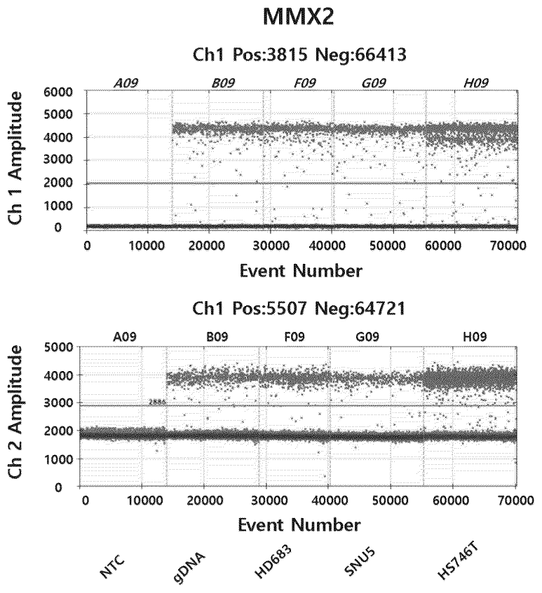

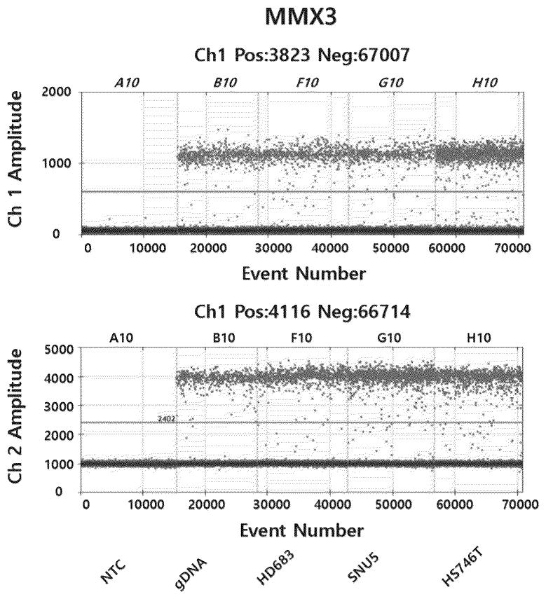

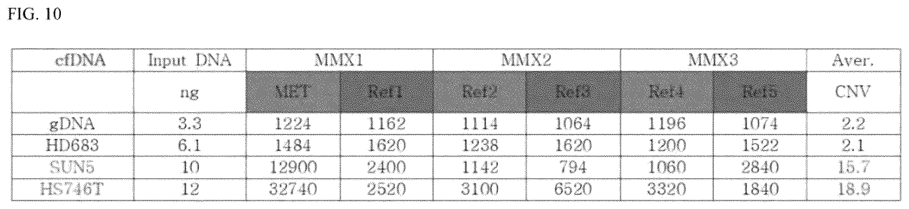

For evaluation of clinical sample applicability, it was confirmed whether the CNV detection kit of the c-MET gene according to the present invention could detect actual CNV even at a size of 200 bp. Through this, it was indirectly confirmed whether CNV could be detected in cfDNA in the blood. The sample used c-Met CNV positive cell lines SNU5 and HS746T, and HD683 was used as a negative sample. DNA fragmentation was performed with an ultra focused sonicator (Covaris). The results of detecting c-Met CNV in the fragmented DNA were illustrated in FIG. 9, and the c-Met copy number and CNV values in the fragmented DNA were illustrated in FIG. 10.

With the above experiment, it was confirmed whether c-MET CNV could be detected in the reference material HD683 cut to 200 bp and the cell line using the ultra focused sonicator. As a result, c-Met CNV was also successfully observed even in DNA fragmented with a size of cfDNA. Therefore, even in cfDNA in the blood below, it was confirmed that c-Met CNV could be detected using the c-Met gene CNV detection kit according to the present invention.

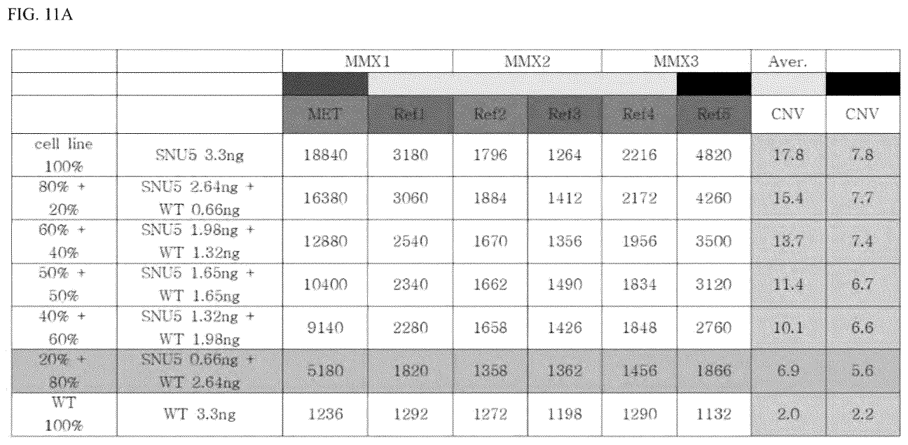

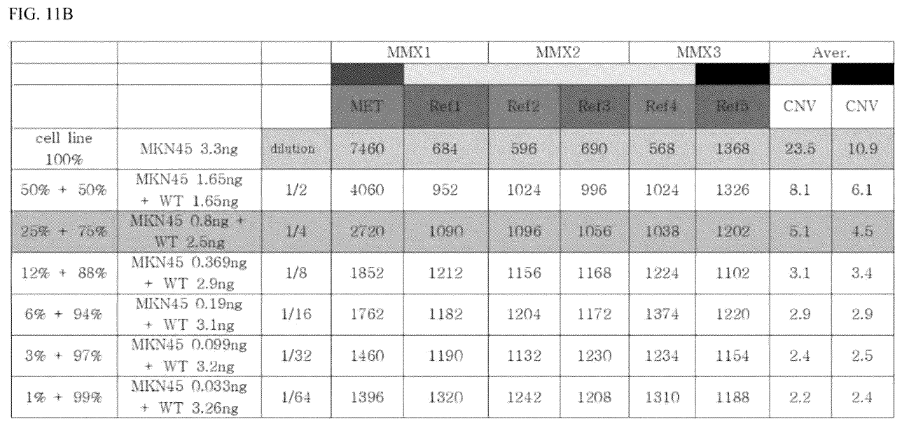

In addition, it was confirmed to what concentration the CNV could be detected when human gDNA was mixed with cell lines SNU5 and MKN45 known to have CNV. The experiment was performed assuming that 3.3 ng=100% of each DNA amount based on 3.3 ng. DNA was extracted from a cell line known to have CNV, the concentration was added according to each %, and the concentration was fixed by adding human gDNA. As a result, as illustrated in FIGS. 11A and 11B, it was confirmed that the degree of determining that there was CNV (CNV>5) was detected up to 20 to 25% when 3.3 ng of the input DNA amount was added.

### INDUSTRIAL APPLICABILITY

As described above, the composition and kit for CNV detection of the c-Met gene according to the present invention can not only detect the CNV of the c-Met gene, but also predict a prognosis of a cancer patient or a risk of mutation occurrence and provide information required for targeted therapy of cancer, so as to be effectively used to provide clues for future treatment directions, as well as to determine a need to administer an anticancer drug, and to monitor the metastasis or recurrence of cancer.

1. A method for selecting reference genes for copy number variation (CNV) detection comprising the steps of:
(a) selecting a normalizing gene candidate group;
(b) obtaining variation information in a target gene in the candidate group and determining whether or not pathogenic variation is present;
(c) calculating a variation rate of the pathogenic variation and selecting the target gene having a variation rate of less than 0.001;
(d) excluding genes corresponding to following criteria from the target genes in the candidate group
(i) the presence of deep deletion, truncating mutation, and missense mutation
(ii) the presence of gene amplification in cancer cells; and
(e) selecting 3 to 6 normalizing genes from the normalizing gene group selected in steps (b) to (d), thereby selecting normalizing genes for copy number variation detection,
wherein at least one of the normalizing genes for copy number variation detection is located on the same chromosome as the target gene to detect the copy number variation.

2. The method of claim 1, wherein the normalizing gene group is a gene group of 18S, ACTB, ALDOA, ARHGDIA, AP3B1, B2M, GAPDH, GUSB, HMBS, HPRT1, HSPCB, IP08, LDHA, NONO, PGK1, POLR2A, PPIA, RPL11, RPL19, RPL32, PLP0, RPS18, RPS27A, EIF2C1, SFRS9, TBP, TFRC, UBC, YWHAZ, RNaseP, TERT, LINE-1, MTHFR, RPPH1, HBB, TUBB, and PPIH.

3. The method of claim 1, wherein the target gene is any one gene selected from the group consisting of c-Met, HER2, HER3, HER4, EGFR, KRAS, NRAS, ALK, RAC1, NTRK1-3, NOTCH1-3, HRAS, HOXA3, DDR2, FGF3, AXIN1, INHBA, RET, FGFR3, MAP2K2, EPHA5, c-Myc, ABL1, ACTN4, AKT1, AKT2, AKT3, APC, ARAF, ARID1A, ARID2, ATM, AXIN1, AXL, BAP1, BARD1, BCL2L11, NRAF, BRCA1, BRCA2, CCND1, CDK4, CDKN2A, CDK4, CRKL, CREBBP, CTNNB, CUL3, ENO1, EP300, ESR1, EZH2, FBXW7, FGFR1-4, FLT3, GNA11, GNAQ, GNAS, ODH1, IDH2, IGF1R, IGF2, IL7R, JAK1˜3, KDM6A, KEAP1, KIT, MDM2, MDM4, MLH1, MTOR, MSH2, NF1, NFE2L2, NRG1, NT5C2, PALB2, PBRM1, PDGFRA, PDGFRB, PIK3CA, PIK3R1, PIK3R2, POLD1, PTCH1, PTEN, RAC1, RAD51C, RAF1, RB1, RET, RHOA, ROS1, SETBP1, SETD2, SMAD4, SMARCA4, SMARCB1, SMO, STAT3, STK11, TP53, TSC1, and VHL.

4. A reference gene set for copy number variation (CNV) detection selected by the method of claim 1.

5. The reference gene set for copy number variation (CNV) detection of claim 4, wherein the reference gene set for CNV detection consists of 3 to 6 genes.

6. The reference gene set for copy number variation (CNV) detection of claim 4, wherein the reference gene set for CNV detection further comprises at least one housekeeping gene.

7. The reference gene set for copy number variation (CNV) detection of claim 6, wherein the housekeeping gene is selected from the group consisting of PRDX6, SULT1A1, BZRP, ATP5G3, CSTB, HNRPF, MAPK3, SNRPC, TGFB1, TCFL1, ALDH9A1, LDHA, PCBP1, KARS, MRPL23, G3BP, ADFP, STOML2, FUSIP1, PLXNB2, KLF6, GPAA1, DKC1, FAM32A, CCNL1, RBM23, TMOD3, TM4SF8, MGC10433, DNCL1, ILK, FLJ20643, MAGED1, ZDHHC4, ADIPOR1, UBA52, C2orf25, RNPEPL1, GNB2L1, CREG1, CNAP1, FBXO7, RAB8A, JTB, RGS19IP1, GPR172A, ATP6AP1, SFRS6, ANAPC5, NOSIP, ATP6V0B, DKFZP566E144, NDUFV1, CALU, FIBP, PNRC2, RYBP, HIG1, RPS20, UQCR, WDR6, TMEM4, DDX42, CANT1, CYR61, FLJ13868, MGC52000, C14orf120, NIFIE14, NME4, C2orf28, SEC11L1, ABCF1, UBQLN1, NDFIP1, CGI-128, DCXR, COPE, RAN, BMS1L, SPCS1, UBE2R2, IDH1, TMPO, UMP-CMPK, ABCE1, TUFM, SNX3, BC-2, WDR39, KIAA1033, SRPRB, BECN1, ADAR, NUP133, NSFL1C, MGC5508, NOLA3, FLJ10349, C10orf9, POLR2G, SLC7A1, TGOLN2, C16orf33, COPS6, RAB7, HSPC0009, E2-230K, KIAA0431, FLJ11730, MGC4767, FUCA2, FLJ12892, PPM1G, LGMN, C20orf44, MRPL15, MAF1, P29, KNS2, CDK5RAP3, HRMT1L2, LOC127262, IGF1R, TIMM17A, DENR, UBE3A, CYBASC3, KIAA0664, LOC92912, FARSLA, SAFB, POLR2E, TRAPPC1, FBLN1, RGS5, NET1, SLC29A1, MTVR1, PFKP, FOXJ3, MGC14156, MAN2C1, GSTZ1, TM4SF7, NOLA2, SAT, APRT, GBL, CLSTN1, MGC2963, HSPC182, TRAP1, PMVK, CKAP1, C20orf14, DKFZP564J0123, CDCA4, TAX1BP1, BLOC1S2, TEGT, MARK3, PPP1R7, HSPH1, STRA13, DPP7, Cab45, MTPN, POLR2I, NDUFA4, HIP2, TEBP, HSPBP1, FAM50A, CD81, MAT2B, RY1, EIF3S7, MRPL51, CTNNAL1, SMC4L1, HSDL2, PTPN12, DDX27, RNF40, BPY2IP1, C22orf16, SFRS1, HEXB, RNF126, UNR, SQLE, MRPS7, CSNK2B, APEX1, GNAI3, SFRS2, ETFB, FVT1, TMP21, DVL1, YWHAQ, GJA1, OAZ2, SSR2, GDI1, TIMM13, MARCKSL1, TSN, FASTK, ILF2, TFAM, ARFIP2, DAP, NDUFA9, BRD2, ARL6IP, IRF3, TUBA1, PSME1, QDPR, ADSL, COPB2, C20orf111, C12orf8, MBTPS1, RNP24, DAG1, ECHS1, UBL4, ZDHHC16, GPX1, GANAB, PSMB6, GNAI2, CDK10, PLP2, HMGN3, USP9X, CSK, SF3A3, HLA-B, DKFZp761I2123, PSMD8, UROD, PGK1, ILVBL, DBI, ADH5, DHPS, PPP1CC, RCN2, CCNG1, NCL, QARS, SMARCD1, PSMC5, POLR2C, RPA2, C20orf149, RPL37, SYPL, ATP5G1, NFKBIA, SUMO1, RAD21, SEC24C, CSNK2A2, GSS, MGC21416, PSMB3, PPP1R11, DAD1, CCT6A, AMPD2, FASN, AAMP, PRDX4, STX4A, SNRPB, DHFR, NDUFA5, GABARAP, FLJ12442, ZFP36L1, ERBP, DERPC, FADD, MSN, PSMB4, TKT, EPHX1, UBTF, SKIV2L, PGRMC1, HSPA4, ADRM1, NDUFS8, KHSRP, MLLT6, ERP70, LOC54499, CDK4, COX11, FLJ21128, HNRPA0, SH3GL1, RCN1, FUBP3, ACTR1B, MCTS1, PSMA1, ARL6IP4, MGC5576, TIMP2, NPDC1, RALGDS, ATP6V0D1, ANAPC13, CCNB1IP1, FLJ31031, C7orf20, C3orf4, SH3BGRL, CSRP1, E2F4, APH-1A, RPS27L, PTD008, SH3BGRL3, C14orf2, SIAT7F, C6orf48, SF3B5, ESRRA, MRPS11, ITM2C, ARS2, SIVA, TOMM7, NUDT5, SSR1, CD9, TXNL1, RFC3, EXOSC7, GLS, PABPN1, ST5, BST2, FSCN1, PNPLA2, NME1, FUT8, p66alpha, GSDMDC1, ARF3, H2AFZ, UQCRC1, AP2S1, RPL3, DNAPTP6, NUP153, CDIPT, WIPI-2, SND1, ARPC1A, FBXL11, C10orf119, BBX, CCT8, EVL, GNAS, AEBP2, RAMP, NAPA, DHX38, EAP30, MAMDC2, RAB5C, CTSC, SEPT11, WDR1, CINP, EIF4A1, TRIO, DDX23, CROP, TM9SF2, BNIP3L, PRNPIP, 132513 HSD17B12, TPM1, SLC35E1, PSMD13, CEBPZ, UREB1, RALY, NCOR2, ARHGAP1, MAEA, M6PRBP1, HP1-BP74, DDX49, DRPLA, S100A10, EBSP, MGC3234, EEF1G, VTI1B, MUF1, ZNF313, SCAMP4, PLEKHM2, MAP2K1, UBL3, TPM3, HERPUD1, QP-C, SPIN, CULL, PCNA, RBAF600, CCM2, ARF4, PTPRG, RHOBTB1, FBXO31, RPS6KA1, PEX14, BIRC6, BC002942, SUI1, TXNDC5, OXA1L, KIAA0992, GMFB, U5-116KD, p44S10, RPS28, TXNDC4, SLC35B1, ZFPL1, HNRPUL1, NFE2L2, KIAA0676, PSMC6, RPS29, KIAA1536, MRPS34, PTD004, H2AFV, HAGH, PRPF4B, AMD1, RAF1, ARHGDIA, FBXO21, THRAP2, CDC37, PDHB, HBP1, CHD4, PACSIN2, MOCS2, UBE2J1, PICALM, VAPA, CTNND1, PHF1, HNRPU, SEC13L1, SFRS5, SRP54, TRPC4AP, METTL3, DIABLO, CNN2, UQCRFS1, NFIC, CNOT7, CFL1, SKP1A, PTBP1, BRP44L, COL1A1, NYREN18, NOC4, DPYSL2, FKBP8, NDUFS2, LOC401152, CKB, EDF1, IFITM2, AK1, YT521, ATP5E, PARP1, RPL8, MBD3, FLOT1, CFL2, MRPS16, HSPA8, H3F3B, 384D8-2, PRDX1, CXXC1, DUSP3, MED4, HMGN2, HLA-A, PRPF8, TMEM9, NHP2L1, C22orf5, SLC35B2, EIF4G2, ADD1, RANGAP1, DCTD, PPP1CA, C20orf24, PMPCB, HSPA9B, ELAVL1, GNB2, SPG7, MALAT1, RPS15A, BRD4, SDFR1, SLC20A1, PAFAH1B2, PLEKHA5, RBM6, NUDT3, PTK9, CXXC5, SMURF1, NDUFAB1, CCT2, GSTO1, MRCL3, RAPlA, COPS4, HSPC142, STRN4, TTC17, SEPT7, DHX9, UBE2J2, CDC2L1, TRA1, EIF3S8, RAI17, BIN1, TUBB6, TCEAL4, ZNF265, FLNA, C17orf27, SSFA2, PKM2, RANBP2, HAX1, HDAC7A, SCAMP3, SDCBP, ch-TOG, WDR45L, GLG1, GK001, VDAC1, HNRPH1, SMN2, KIAA0261, SGTA, AHSA1, MEP50, MRPL3, CTTN, MGC71993, CTBP1, STMN1, ELMO2, KIAA0141, DNM2, PSMC4, NDUFS7, TXNDC7, CIZ1, NUCKS, PSMB7, KIAA0460, SUPT16H, FBXO9, FOXO3A, SLC38A2, DAZAP1, DDX21, SDC1, RDH11, H1F0, YWHAH, ATAD3A, TOMM70A, PTP4A1, PC4, PITPNC1, HSPC023, KIAA0174, PDCD4, NFYC, CDC2L5, PSMA7, MAPKAPK3, SMARCC2, MGC20781, XTP3TPA, SCYL1, MGC2731, ATPIF1, POLDIP2, ARIH2, GNPTG, DERL1, SERPINH1, SPG21, DGKI, ASCC3L1, ATP5J, CPNE1, FBXO11, RHOA, FBS1, HSPD1, MPST, TAF9, DLGAP4, ARL10C, SUPT6H, PSMC3, HSBP1, LOC51234, PSMA4, MVD, TTC15, DKFZp566C0424, PAPOLA, STOM, BAT1, VPS24, PFKL, XRN2, BTG1, CRI1, MGC13170, NUBP2, PLD3, SAV1, SH3BP5, DNPEP, FEZ2, C10orf86, PALM2-AKAP2, PIP5K2B, FLJ10326, ITGA3, KIAA1185, GPS1, C13orf12, GLO1, MATR3, MAK3, PTPRA, GNAQ, MTCH2, ACTN4, SUCLG1, LASS5, ZNF410, ATP5C1, NOB1P, PTPRF, TDE1, ATP6V1D, SEC23A, TMEM14B, TFE3, C15orf15, CARS, S100A6, SEPP1, PCNP, NUP93, MGLL, C11orf2, ARHGEF1, MEA, PDAP1, C9orf78, GNPDA1, SNX17, CD59, SLC39A7, C17orf25, TACC1, PCMT1, POLD1, PX19, DREV1, SEPX1, TPR, MRPL4, TUBG1, SUMF2, DDX5, COMMD9, YWHAB, TMED9, SBF1, PRKARIA, SNRPB2, CALM1, SPCS2, RNPC2, RBM9, C14orf124, BNIP2, RHEB, APG4B, IDI1, UBQLN4, ANKRD25, HMOX2, MRPS24, PDXK, MAX, LASS2, ARF1, MYO1C, KPNA4, PFDN5, HSCARG, C6orf68, COTL1, DCTN2, CYC1, GBF1, SLC25A3, DDB1, ARID1B, EIF4E2, EIF4B, LARP, ZMYND11, 292457, G22P1, CCNH, PTDSS1, FLJ12666, ATP6V1B2, TIMP3, CKLFSF3, ATP5A1, DUSP6, FBL, GDI2, RPL39, RCP9, TPM2, 300816 RABIB, GALNT2, RBM3, Ufc1, MRPS6, UBE2I, G3BP2, DSTN, CST3, MAGEF1, RANBP9, ZA20D3, IBTK, ITPK1, NUP188, GRP58, SFRS7, C6orf153, RNF11, STARD3NL, C14orf159, TOMM40, RAB1A, MRPS35, CMAS, DDX39, RPS27A, ADAM15, TXNDC11, BAZ2A, EIF3S12, IFRD2, GC20, NASP, MGC4549, RAB11A, APG9L1, FLJ20758, NDUFB2, VKORC1, EHD2, MORF4L2, CORO1C, SCC-112, EEF1D, HSPC152, PSMA2, MRPL13, K-ALPHA-1, TRAF7, GNS, RBPMS, BMSC-UbP, LASP1, PPP2R5E, ANKRD11, SEPT2, KIAA1102, FDPS, STIP1, TXNRD1, COPB, COX7A2L, GRINA, E124, KCMF1, EBNAIBP2, DR1, SCARB2, ZMAT2, CASC3, IRF2BP2, C9orf100S, SLC25A6, FLJ10241, LOC51035, TM4SF1, PAQR4, COX6C, STCH, GHITM, PSMB1, POR, TNIP1, MGC23909, RNPS1, FLJ20507, MGC5306, DKFZp586M1819, MARS, VDAC2, SFPQ, BZW1, MAP1LC3B, FLJ10350, UBB, SDHD, HMGN1, PPIA, RPS2, RPL28, LOC149603, MGC2747, PHF6, RPLP1, SNRPD3, NUTF2, SNX6, PSMC1, MAN2B1, SOX4, SEP15, U2AF1, LRPPRC, CCT7, PYGB, DYRKIA, PPP2R5C, SRPR, LOC51337, EXT2, PDLIM1, LEREPO4, MGC40157, TRIP12, RAB2, SELT, DNCLI2, PSMD14, DKFZP434B168, MLL5, CPSF6, GAK, COPS2, FLJ20551, DAZAP2, MGC2749, RAP1B, SEC31L1, APLP2, BCCIP, FNTA, COMT, CAP1, XPO1, CDKN1A, RPN2, PRO1855, TAPBP, EIF3S9, CARM1, RAB14, DKFZP547E1010, ATP1A1, C9orf10, SMAP-5, CUL3, SPTAN1, KBTBD2, ARL1, NDRG1, TGIF, HM13, HNRPR, CAMTA2, VGLL4, ASXL1, SIAT4A, CKS1B, EWSR1, MORF4L1, RPL17, TPT1, IFITM3, PRPF4, TLN1, CD24, RNF31, BTN3A2, GUK1, LYRIC, RPS5, HBS1L, eIF2A, PCGF4, DDX3X, RPL38, SUMO2, HLA-E, KIAA0146, PPIF, RPL21, RPS14, CBX3, 381219, GLTP, MRPS2, NCOA3, ANP32E, UBE2B, ANXA7, CHERP, USP7, FAU, PABPC1, RXRB, ATP6V1G1, RPL11, XRCC5, YY1, C19orf22, MCM3APAS, ATP6V0C, PINK1, DDX48, TCEAL8, CHCHD2, GSTK1, ACTR2, PTK2, PAIP2, NIP30, SLC9A3R1, RPS16, AKAP8L, RPL30, RBM10, COX5A, RPL10, STK24, EIF3S1, GARS, SFRS3, OGT, LOC284058, EIF3S6, MRPS21, LOC339229, NDUFA11, UBE2M, ZA20D2, SF3A1, RPL23, SF3B2, ATP5B, LOC389541, HMG20B, PGR1, RPS10, RPS15, RAB18, SLC35A4, C19orf13, PKP4, MIF, RPL36, RPS6, TXNL5, NDUFS6, KAB, FLJ10769, CHES1, SVIL, GOLPH3, ATP50, SSR4, AGPAT1, PHPT1, IDH3G, HAN11, RPL13, AUP1, EIF4EBP1, MAPK6, EFHA1, ANXA6, ESRRBL1, AIP, KLHDC3, C10orf7, WBSCR22, C21orf33, SCOTIN, KIAA1049, TNPO2, TRIM50A, S100A11, DERP6, CTSL, VPS28, MRPL24, MRPL11, BUB3, ATP5D, PARK7, COL6A2, H2AFY, RPL7, CCT4, ZNF511, RDBP, TTC11, SERF2, LSM5, UBE2L6, C10orf104, DKFZp564J157, ITGB1, SEPN1, RTN4, PITPNA, MGC23908, GNB1, IQGAP1, CS, ARF5, CLNS1A, GNG12, C6orf55, FOXP1, MAP4K4, COX6B1, MAPK1, PPP5C, NFKB1, PRDX2, EML4, ESD, SLC39A9, CAPZB, RPL4, NR1H2, PLSCR3, CDK2AP1, NPC2, ARD1, BCKDHA, SRRM2, 433345, COX4I1, ACTR3, RPS11, DNAJC8, Bles03, TUBB2, RPL37A, KIAA1967, CLK1, EIF4G1, BANF1, SHC1, PBP, COX8A, GPX4, HMGB1, HARS2, ANKHD1, ZNF638, PPIB, HMGB2, APP, TBC1D22A, KIAA1608, KIF1C, TXN, LBR, ZFR, UBXD1, ACTL6A, PPP3CA, ZNF395, WAC, GCSH, THAP4, API5, TNRC15, LYPLA1, PHF10, ATAD1, CDK5RAP1, MTHFD1, TUBA6, BAT2, ZNF289, EMP1, IDH3B, ALDH2, ARMET, DBNL, CD74, POLR2F, CLU, SET, VBP1, SUPT5H, CYCS, ANXA2, ACADVL, GRINL1A, MGAT4B, GBAS, PIGT, PP, RPLP2, XBP1, C11orf10, C14orf32, UNC84A, GPS2, RPS19, TALDO1, MCM7, TBL1XR1, CUTL1, RBM5, SUPT4H1, FLJ22875, 439552, HBXIP, RFP, CLIC4, DDX1, PSMD7, TTYH3, SEPT9, RAD23A, CAST, POLR2L, C20orf22, IRS2, RNF10, GBA2, PSMD11, NPEPPS, SOD1, DKFZp761C169, GRB2, CTDSP1, SDHC, VMP1, CRR9, AZI2, CGGBP1, CRSP6, C2orf4, METAP2, NS5ATP13TP2, LGALS1, VPS29, SKIIP, RIOK3, CD63, DC2, KHDRBS1, GTF3A, WSB1, WTAP, CAPZA2, LDHB, PSMA6, PXN, FTH1, CD47, OAZ1, YIF1, ITM2B, TMSB10, RPS13, HNRPL, RPS4X, ARAF, EIF3S6IP, ST13, PGAM1, VPS35, RPLP0, NGFRAP1, RPL27A, HNRPC, HNRPK, USF2, IL6ST, PKD1-like, FLJ10597, DC12, TSPYL1, IFITM1, C19orf27, ANP32A, OAZIN, BTBD1, FLJ23790, AKAP13, MPG, CLCN7, PTMA, LITAF, SH3GLB2, ALS4, GGA2, XPO6, ATXN2L, LOC124446, CNOT1, GOT2, APPBP1, G6PD, CYB5-M, CFDP1, GABARAPL2, HSPC176, PCOLN3, CRK, RPA1, UBE2G1, RIP, UBE2S, TTC19, USP22, PIGS, PPARBP, IGFBP4, SMARCEl, FKBP10, RERE, STAT3, CDC27, AKAP1, BCAS3, C1orf33, PGD, ACOX1, SYNGR2, P4HB, AGTRAP, MRLC2, PPP4R1, TNFSF5IP1, P15RS, NARS, EFHD2, TXNL4A, MIDN, BTBD2, MAP2K2, C19orf10, HNRPM, PIN1, SDHB, PKN1, TPM4, NR2F6, GPI, SIRT2, LTBP4, SNRPA, EIF4G3, SNRP70, PPP2R1A, LENG4, RPS9, TRIM28, CDC42, HPCAL1, ODC1, LAPTM4A, PUM2, RAB10, PPP1CB, PIGF, CALM2, AFTIPHILIN, DGUOK, DKFZP564D0478, STARD7, RALB, YWHAZ, FLJ14346, SFRS4, YWHAE, ARL5, 470417, PTP4A2, EIF2S2, KPNA6, STAT1, SF3B1, NOP5/NOP58, NDUFS1, PSMB2, ACSL3, CAB39, MGC4796, M11S1, HDLBP, DTYMK, FKBPIA, C20orf116, PRNP, C20orf30, UBE2D3, CGI-94, SYNCRIP, MKKS, NDUFS5, RRBP1, C20orf3, MACF1, AKIP, SDBCAG84, BLCAP, TOP1, TPD52L2, NSEP1, GART, SLC2A1, RTN3, OTUB1, SUMO3, PITG1IP, COL6A1, B4GALT2, UFD1L, AKR1A1, HSPC117, MYH9, CSNKIE, RUTBC3, SLC25A17, RBX1, ACO2, ATXN10, LRRFIP2, FLJ22405, LOC55831, RAB5A, TOP2B, SIMP, CTNNB1, TLP19, SMARCC1, IHPK2, IMPDH2, ALAS1, SCP2, FLNB, MRPL37, DKFZP5640123, DULLARD, ATP1B3, GYG, MBNL1, PA2G4, EIF4A2, FBXL5, LAP3, SLC30A9, SFRS11, GAPD, BCAR1, POLR2B, HNRPD, PDLIM5, SCYE1, ANXA5, UQCRH, MYO10, KAT3, RPL26, SLC30A5, TINP1, KIAA0372, PJA2, C5orf13, HINT1, PPP2CA, CNN3, JMJD1B, FBXW11, ATP6V0E, ETEA, DDX41, RNF130, CPM, DEK, RPL35, SORT1, PSMA5, MTCH1, AHCYL1, DST, BCLAF1, VIL2, TCP1, BZW2, HNRPA2B1, KIAA1068, OGDH, DKFZP564K0822, FLJ10099, BAZ1B, ASNS, ARPCIB, CPSF4, SYAP1, PBEF1, CALD1, SSBP1, ZYX, DNAJB6, CHR2SYT, MTX1, ELP3, LMNA, PPP2CB, CCT3, VDAC3, UBE2V2, TCEA1, TRAM1, WDR42A, LAPTM4B, EDD, EIF3S3, CGI-07, AK3L1, WDR40A, ANXA1, LAMP1, NINJ1, ANP32B, XTP2, PFN1, CDW92, FBXW2, NDUFA8, KIAA0515, PMPCA, CD99, MGC4825, ATP6AP2, PCTK1, DKFZp761A052, 496271, ATF4, IL13RA1, LAMP2, IVNSIABP, WARS, C1orf48, ENAH, FH, ADSS, PNAS-4, KIAA0217, RBM17, DHCR24, YME1L1, GGA1, TIMM23, C10orf74, HNRPH3, VPS26, SARA1, PPP3CB, VCL, ENTPD6, GLUD1, IDE, SMBP, ZNF207, MGEA5, CUEDC2, ADD3, MXI1, TIAL1, BSG, CIRBP, PLEKHJ1, UROS, NCLN, IER2, NAP1L4, STIM1, C11orf15, USP47, MLSTD2, CAT, CD44, DGKZ, NDUFS3, C11orf31, RHOC, PRP19, FADS2, SLC3A2, MTCBP-1, PRDX5, SF1, ARL2, CAPN1, MAP3K11, RHOB, ZFP36L2, RAB6A, PME-1, HSPC148, PORIMIN, MGC2714, DARS, ITM1, RPS27, PTMS, REA, MYL9, DDX47, STRAP, KRAS2, PSMD4, C12orf10, COPZ1, CIP29, MYL6, PBXIP1, CGI-51, RARS, NUDT4, ATP2A2, DDX54, KIAA0152, FLJ12750, MGC9850, PFAAP5, PGRMC2, TGFB1I4, FNDC3A, FLJ10154, KIAA1181, FKBP3, KLHDC2, KTN1, RGL2, HSPCB, ERH, NUMB, ENSA, DDX24, MCP, FLJ10579, DKFZP564G2022, SNAP23, SRP9, TCF12, 511862, CBX6, ARPC3, SURF4, RPS17, MIR16, PRKCSH, KIAA1160, 512676, FLJ20859, THAP7, AP3D1, CD151, H63, ARPP-19, C15orf12, REC14, RANBP5, TMED3, MESDC1, RPL9, IDH2, NEUGRIN, FURIN, MRPL28, RHOT2, C16orf34, NDUFB10, NFATC2IP, MVP, ALDOA, BCKDK, FUS, ARL2BP, DPH2L1, FLII, MAP2K3, SDF2, FLOT2, JUP, RPL27, MGC4251, CGI-69, GRN, FLJ13855, PHB, SF3B3, WBP2, LGALS3BP, ACTG1, HGS, AP2B1, ATP5F1, NDP52, CAPZA1, C19orf6, STK11, GNA13, AES, EEF2, CLPP, MGC2803, CALR, GADD45GIP1, DNAJB1, LSM4, RENT1, SFRS14, RPL22, CAPNS1, AKT2, EGLN2, DEDD2, SNRPD2, SYMPK, CALM3, SLC1A5, SAE1, KDELR1, RPL18, NUCB1, PTOV1, LOC284361, PRPF31, PPP1R12C, GPSN2, BLVRB, RUVBL2, HADHB, YPEL5, TIA1, FLJ14668, TEX261, DCTN1, WBP1, MAT2A, FLJ20297, FLJ21919, HNRPA3, UBE2Q, NCKAP1, CHPF, ARHGEF2, STK25, TRIB3, CENPB, SLC35C2, CEBPB, C20orf43, EN01, TAGLN2, PEA15, PEX19, IFNGR2, SON, F11R, ATP6V1E1, DEDD, COL18A1, DGCR2, PCQAP, ASCC2, EP300, PES1, MCM5, UNC84B, L3MBTL2, DIA1, PP2447, DKFZP564B167, C3orf10, MGC3222, 517821, CRTAP, DHX30, MAP4, APEH, TUSC2, ARL6IP5, TFG, SEC61A1, RPN1, ZNF9, COPG, H41, SERP1, SSR3, QSCN6, NDUFB5, AP2M1, PSMD2, GLUL, RPL31, PP784, ARPC5, OCIAD1, HMGA1, CCNI, MAPKAPK2, PELO, ERBB2IP, SFRS12, RPS25, SERPINB6, TMEM14C, TTC1, STK10, MGAT1, MARCKS, C6orf62, VARS2, HSPA1A, NEU1, C6orf82, SRF, ELOVL5, EIF2AK1, KDELR2, C6orf111, CD164, STX7, PERP, GTF2I, C7orf27, ACTB, SCRN1, YKT6, CTSB, WBSCR1, MDH2, HSPB1, YWHAG, ZNF655, FLJ22301, REPIN1, MGC8721, RAD23B, COBRA1, LY6E, SIAHBPI, NDUFB11, WDR13, DSIPI, CREB3, CLTA, NANS, GSN, HSPA5, EEF1A1, FBXW5, TMSB4X, EIF1AX, TIMP1, MAGED2, FLJ12525, PSMD10, BCAP31, IRAK1, EMD, NCOA4, EIF4EBP2, PSAP, DDIT4, SMP1, TRAPPC3, DDOST, NDUFB8, NOLC1, C1orf8, EIF3S10, PRDX3, HSPCA, SSRP1, TncRNA, POLD4, GSTP1, CCND1, INPPL1, AASDHPPT, DPAGT1, RNF26, RSU1, RAD52, FKBP4, ARHGAP21, MLF2, TPI1, PHC2, CBARA1, TUBA3, ATP5G2, RNF41, CTDSP2, RAB21, NAP1L1, PPIE, RAB35, RSN, SAP18, ZFP91, Ufm1, FLJ20277, ANKRD10, LRP10, C14orf119, ARF6, FLJ20580, RER1, PACS1L, C6orf49, PML, MDH1, HNRPDL, RPS23, AKAP9, ASAH1, OS-9, PKD1, DUT, HARS, NDUFS4, PITRM1, DDX17, SCAM-1, RPL6, GSPT1, UQCRC2, EIF3S4, SEPW1, NCK2, ANAPC7, ARPC2, AFAP, IFNAR1, UBE2G2, TLOC1, TFRC, RPL35A, VCP, BTF3, CSNK1A1, CANX, SQSTM1, SEC63, EIF3S2, LUC7L2, ANXA11, SSNA1, PDHA1, PIM3, PAI-RBP1, STXBP3, PMF1, RNH, MRPL16, FLJ20625, COPS7A, PRKAG1, LGALS3, PSMA3, SARS, CBWD1, BTBD14B, RANBP3, GAS5, DNCL2A, RAD1, RPL5, KIAA0663, GTL3, NMT1, KPNB1, HN1, MCL1, NDUFB7, HRIHFB2122, TUBB, SFRS10, LRPAP1, TOMM20, HSA9761, DDX46, NONO, PPP2R5D, VIM, TCEB1, WWP1, PPP1R8, LYPLA2, ATRX, H3F3A, RBM4, SRP14, STUB1, KRT8, TXNIP, EXOC7, ZNF258, HLA-C, NDUFA1, SEC22L1, B2M, CCND3, EIF5A, ITGB4BP, PPP4C, RPS7, SMARCB1, GRIM19, ANAPC11, C14orf166, TOMM22, MGC2941, PACS1, NDUFB9, CTSD, DNCI2, FDFT1, HNRPA1, RPL10A, PCBP2, RPS3, RPS12, RPS18, NET-5, SMAP, RPL13A, HSPC016, SSBP3, C9orf86, HTF9C, PGM3, C3F, NUP88, SREBF1, FLJ12953, MGC11257, FAM36A, C1QBP, HNRPAB, IDH3A, PSMC2, RBBP4, RBM8A, RIC8, PP1201, MRPS25, LONP, FBXL10, NDUFV2, PAICS, NPM1, ACP1, COX6A1, EEF1B2, EIF5, FKBP5, FTL, HSPE1, IK, LAMR1, NDUFB4, NME2, NUMA1, RAC1, RNU65, RPL15, RPL18A, RPL19, RPL23A, RPL34, RPS3A, RPS8, RPS24, RPS26, SCD, CSDA, EIF3S5, G10, RPL14, PDCD6IP, TXNL2, ATP5L, NUDC, COPS8, C18orf10, 8D6A, PSARL, C2orf33, ORMDL1, PDE4DIP, CD2BP2, C1orf43, ATP2C1, and HCNGP.

8. A method for determining copy number variation of a target gene for copy number variation detection comprising the steps of:
(a) measuring the copy numbers of a target gene for copy number variation detection and the reference gene set of claim 4 in a sample;
(b) calculating a mean value of the copy numbers of each gene in the reference gene set; and
(c) calculating a value by dividing the copy number of the target gene for copy number variation detection by the mean value calculated in step (b),
wherein the value calculated in step (c) is compared with 2 to determine whether there is the copy number variation.

9. The method of claim 8, wherein the target gene for copy number variation detection is any one selected from the group consisting of c-Met, HER2, HER3, HER4, EGFR, KRAS, NRAS, ALK, RAC1, NTRK1-3, NOTCH1-3, HRAS, HOXA3, DDR2, FGF3, AXIN1, INHBA, RET, FGFR3, MAP2K2, EPHA5, c-Myc, ABL1, ACTN4, AKT1, AKT2, AKT3, APC, ARAF, ARID1A, ARID2, ATM, AXIN1, AXL, BAP1, BARD1, BCL2L11, NRAF, BRCA1, BRCA2, CCND1, CDK4, CDKN2A, CDK4, CRKL, CREBBP, CTNNB, CUL3, ENO1, EP300, ESR1, EZH2, FBXW7, FGFR1-4, FLT3, GNA11, GNAQ, GNAS, ODH1, IDH2, IGF1R, IGF2, IL7R, JAK1-3, KDM6A, KEAP1, KIT, MDM2, MDM4, MLH1, MTOR, MSH2, NF1, NFE2L2, NRG1, NT5C2, PALB2, PBRM1, PDGFRA, PDGFRB, PIK3CA, PIK3R1, PIK3R2, POLD1, PTCH1, PTEN, RAC1, RAD51C, RAF1, RB1, RET, RHOA, ROS1, SETBP1, SETD2, SMAD4, SMARCA4, SMARCB1, SMO, STAT3, STK11, TP53, TSC1, and VHL.

10. The method of claim 8, wherein the mean value of step (b) is an arithmetic mean value.

11. The method of claim 8, wherein in the determining of whether there is the copy number variation, it is determined that there is copy number variation when the calculated value in step (c) is greater than 2, and it is determined that there is no copy number variation when the calculated value is 2 or less.

12. The method of claim 8, wherein the reference gene set includes a gene located in an identical chromosome to the target gene for copy number variation detection and a gene located in a non-identical chromosome, and
a value obtained by dividing the copy number of the target gene for copy number variation detection by the copy number of the gene located in the same chromosome is 1.5 to 2.5, wherein when the value obtained by dividing the copy number of the target gene for copy number variation detection by the copy number of the gene located in the non-identical chromosome is greater than 2, it is determined that there is the copy number variation, and when the value is 2 or less, it is determined that there is no copy number variation.

13. A primer and probe set composition for c-Met copy number variation (CNV) detection comprising:
at least one polynucleotide set as an active ingredient selected from the group consisting of i) a polynucleotide set for c-Met detection consisting of a primer of SEQ ID NO: 7, a primer of SEQ ID NO: 8, and a probe of SEQ ID NO: 1; and
ii) a polynucleotide set consisting of a primer of SEQ ID NO: 9, a primer of SEQ ID NO: 10, and a probe of SEQ ID NO: 2; a polynucleotide set consisting of a primer of SEQ ID NO: 11, a primer of SEQ ID NO: 12, and a probe of SEQ ID NO: 3; a polynucleotide set consisting of a primer of SEQ ID NO: 13, a primer of SEQ ID NO: 14, and a probe of SEQ ID NO: 4; a polynucleotide set consisting of a primer of SEQ ID NO: 15, a primer of SEQ ID NO: 16, and a probe of SEQ ID NO: 5; and a polynucleotide set consisting of a primer of SEQ ID NO: 17, a primer of SEQ ID NO: 18 and a probe of SEQ ID NO: 6.

14. The primer and probe set composition of claim 13, wherein the c-Met CNV detection is to evaluate prognosis of cancer or predict a risk of mutation occurrence.

15. The primer and probe set composition of claim 13, wherein the c-Met CNV detection is to provide information required for targeted therapy of cancer.

16. The primer and probe set composition of claim 14, wherein the cancer is one or more selected from the group consisting of breast cancer, cervical cancer, cholangiocarcinoma, colorectal cancer, colon cancer, endometrial cancer, esophageal cancer, stomach cancer, head and neck cancer, kidney cancer, liver cancer, lung cancer, nasopharyngeal cancer, ovarian cancer, pancreatic cancer, gallbladder cancer, prostate cancer, thyroid cancer, osteosarcoma, rhabdomyosarcoma, synovial sarcoma, Kaposi sarcoma, leiomyosarcoma, malignant fibrous histiocytoma, fibrosarcoma, acute myeloid leukemia, adult T-cell leukemia, chronic myeloid leukemia, lymphoma, multiple myeloma, glioblastoma, astrocytoma, melanoma, mesothelioma, Wilms' tumor, and MiT tumor including clear cell sarcoma (CCS), alveolar soft part sarcoma (ASPS), and translocation-associated renal cell carcinoma.

17. The primer and probe set composition of claim 13, wherein the probe is bound with a fluorescent material.

18. The primer and probe set composition of claim 17, wherein the fluorescent material is one or more selected from the group consisting of VIC, HEX, FAM, and EverGreen dyes.

19. A kit for detecting c-Met gene copy number variation (CNV) comprising the primer and probe set composition according to claim 13 as an active ingredient.

20.-26. (canceled)

27. A method for detecting c-Met copy number variation (CNV) comprising the steps of:
(a) administering, to a target gene for CNV detection in a sample, at least one polynucleotide set from the group consisting of
i) a polynucleotide set for c-Met detection consisting of a primer of SEQ ID NO: 7, a primer of SEQ ID NO: 8, and a probe of SEQ ID NO: 1; and
ii) a polynucleotide set consisting of a primer of SEQ ID NO: 9, a primer of SEQ ID NO: 10, and a probe of SEQ ID NO: 2; a polynucleotide set consisting of a primer of SEQ ID NO: 11, a primer of SEQ ID NO: 12, and a probe of SEQ ID NO: 3; a polynucleotide set consisting of a primer of SEQ ID NO: 13, a primer of SEQ ID NO: 14, and a probe of SEQ ID NO: 4; a polynucleotide set consisting of a primer of SEQ ID NO: 15, a primer of SEQ ID NO: 16, and a probe of SEQ ID NO: 5; and a polynucleotide set consisting of a primer of SEQ ID NO: 17, a primer of SEQ ID NO: 18 and a probe of SEQ ID NO: 6;
(b) measuring the copy number of the reference gene set of claim 4;
(c) calculating a mean value of the copy numbers of each gene in the reference gene set;
(d) calculating a value by dividing the copy number of the target gene for copy number variation detection by the mean value calculated in step (c); and
(e) comparing the value calculated in step (d) with 2 and determining that the copy number variation exists when the value is greater than 2.
